(19) United States
(12) Patent Application Publication
(10) Pub. No.: US 2015/0331992 A1
(43) Pub. Date: Nov. 19, 2015

(54) CANCER PROGNOSIS AND THERAPY BASED ON SYNTHEIC LETHALITY

(71) Applicant: Ramot at Tel-Aviv University Ltd., Tel Aviv (IL)

(72) Inventors: Livnat ARNON JERBY, Tel Aviv (IL); Eytan RUPPIN, Modin-Macabim-Reut (IL)

(21) Appl. No.: 14/712,256

(22) Filed: May 14, 2015

Related U.S. Application Data
(60) Provisional application No. 61/993,287, filed on May 15, 2014.

(51) Int. Cl. G06F 19/18; A61K 31/55; A61K 31/343; A61K 31/137; A61K 31/277; A61K 31/4025; A61K 31/4409

(52) U.S. Cl. CPC G06F 19/18; A61K 31/4025; A61K 31/55; A61K 31/4409; A61K 31/137; A61K 31/277; A61K 31/343

(57) ABSTRACT

Systems and methods for identifying synthetic lethal (SL) and synthetic dosage lethal (SDL) interactions and networks are provided. Further provided are methods for predicting cancer gene essentiality, drug efficacy and survival of cancer patients using data-driven identification of synthetic lethality in cancer are provided. Novel drug candidates and drug combinations for use in cancer therapy and method for prioritizing existing cancer therapies are also provided.

### FIELD OF THE INVENTION

The invention is in the field of bioinformatics, cancer research and personalized medicine and provides systems and methods for identifying synthetic lethal (SL) and synthetic dosage lethal (SDL) gene pair interactions and networks. Also provided are methods for predicting drug responses and selection of candidate drugs for cancer therapy.

### BACKGROUND OF THE INVENTION

Synthetic lethality occurs when the perturbation of two nonessential genes is lethal (Hartwell et al., 1997). This phenomenon offers a unique opportunity to develop selective anticancer drugs that will target a gene whose Synthetic Lethal (SL)-partner is inactive only in the cancer cells (Ashworth et al., 2011; Hartwell et al., 1997; Vogelstein et al., 2013). Towards the realization of this potential, screening technologies have been developed to detect SL-interactions in model organisms (Byrne et al., 2007; Cokol et al., 2011; Costanzo et al., 2010; Horn et al., 2011; Typas et al., 2008) and in human cell lines (Barretina et al., 2012; Bassik et al., 2013; Bommi-Reddy et al., 2008; Brough et al., 2011; Garnett et al., 2012; Iorns et al., 2007; Laufer et al., 2013; Lord et al., 2008; Martin et al., 2009; Turner et al., 2008). However, their scope of is not sufficiently broad to encompass the large volume of genetic interactions that need to be surveyed across different cancer types.

Previous computational approaches developed to systematically study genetic interactions have mainly focused on yeast, where there are genome-wide maps of experimentally determined SL-interactions (Chipman and Singh, 2009; Kelley and Ideker, 2005; Szappanos et al., 2011; Wong et al., 2004). In cancer, synthetic lethality has been computationally inferred by mapping SL-interactions in yeast to their human orthologs (Conde-Pueyo et al., 2009; O'Neil et al., 2013), and by utilizing metabolic models and evolutionary characteristics of metabolic genes (Folger et al.; Frezza et al., 2011; Lu et al., 2013). Jerby et. al., 2014, discloses predicting cancer-specific vulnerability via data-driven detection of synthetic lethality.

US 20120208706 discloses a method of analyzing a tumor sample for mutations.

US 20130323744 provides methods of predicting the presence of a tumor in a subject by analyzing a subject sample to obtain a subject gene expression profile and comparing the subject gene expression profile to a KRAS activation profile, wherein a similarity of the subject gene expression profile and the KRAS activation profile indicates the presence of a tumor in the subject.

US 20130260376 utilizes gene expression profiles in methods of predicting the likelihood that a patient's cancer will respond to standard-of-care therapy and methods of identifying therapeutic agents that target cancer stem cells or epithelial cancers that have undergone an epithelial to mesenchymal transition using such gene expression profiles.

There is an unmet need for new bioinformatics approaches to boost the experimental search for SL-interactions in cancer and identify better treatment strategies.

### SUMMARY OF THE INVENTION

The present invention provides, in some embodiments thereof, systems and methods for identification of Synthetic Lethal (SL)-interactions and networks and/or Synthetic dosage Lethal (SDL)-interactions and networks and uses of such identified interactions and networks for various applications, including but not limited to cancer related applications.

According to some embodiments, the systems and methods disclosed herein provide data-driven computational systems and methods for the genome-wide identification and utilization of candidate Synthetic Lethal (SL)-interactions and networks and/or Synthetic dosage Lethal (SDL)-interactions and networks in cancer, by analyzing large volumes of cancer genomic profiles. The approach, designated the DAta-mIning SYnthetic-lethality-identification and utilization pipeline (DAISY), has been comprehensively tested and validated, and its superiority compared to other methodologies has been shown. DAISY first generates genome-scale SL-networks and then applies these networks as a platform for various clinical and commercial applications in the field of cancer research and pharmacology. By implementation of its SL-networks it enables the user to tackle five main challenges: (1) Tailoring personalized treatments for patients based on the genomic profiles of their tumors, focusing on three therapeutic criteria: efficacy, selectivity, and low chances for the emergence of drug resistance; (2) Drug repurposing—identifying drugs, which are currently used to treat other diseases (not cancer) as an effective treatment against specific cancer types; (3) Rational drug target identification—identifying genes whose inhibition is selectively lethal to cancer cells of various tumors, and not to healthy cells, to develop drugs that will target these genes; (4) Identification of synergistic drug combinations in cancer by detecting non-essential genes that participate in SL-interactions which are manifested only in cancer and not in healthy cells; and (5) Cancer prognosis prediction based on the cancer genetic profile.

In some embodiments, the present invention provides a system for identifying Synthetic Lethal (SL) interactions of pairs of genes in cancer cells, the system comprising:
a non-transitory computer readable memory having stored thereon datasets comprising data related to multiple genes in said cancer cells, and
a processing circuitry configured to recursively:
   select a pair of genes comprising a first gene (A) and a second gene (B) from the multiple genes datasets;
   analyze the pair of genes to determine the association of said pair of genes, wherein the association is determined by one or more of the following procedures:
      examine if an occurrence of co-inactivation in the cancer cells of the first gene and the second gene is lower than a predetermined threshold;
      determine if the essentiality of the second gene (B) is higher in the cancer cells in which the first gene (A) is inactive; and/or
      determine if the expression of the first gene and the second gene correlate with cancer;
      and;
   determine, based on said analysis, if the pair of genes interact via an SL-interaction, and/or determine the strength of the SL-interaction.

According to some embodiments, there is provided a system for identifying Synthetic Dosage Lethal (SDL)-interactions of pairs of genes in cancer cells, the system comprising:
a non-transitory computer readable memory having stored thereon datasets comprising data related to multiple genes in said cancer cells, and
a processing circuitry configured to recursively:
   select a pair of genes comprising a first gene (A) and a second gene (B) from the multiple genes datasets;
   analyze the pair of genes to determine an association of said pair of genes, wherein the association is determined by one or more of the following procedures:
      examine if an occurrence of over activation in the cancer cells of the first gene and inactivation of the second gene is lower than a predetermined threshold;
      determine if the essentiality of the second gene (B) is higher in the cancer cells in which the first gene (A) is overactive; and/or
      determine if the expression of the first gene and the second gene correlate with cancer;
      and;
   determine, based on said score, if the pair of genes interact via an SDL-interaction, and/or determine the strength of the SDL-interaction.

In some embodiments, the data related to the multiple genes may be selected from activity profile of the genes, essentiality profile of the genes, expression profile of the genes, or combinations thereof.

In some embodiments, the activity profile of the genes is selected from or comprises Somatic Copy Number of Alterations (SCNA), germline Copy-Number Variations (CNV), DNA methylation, histone methylation, somatic mutations, germline mutations or combinations thereof. In some embodiments, the activity profile of the genes may be obtained from a source selected from the group consisting of: a sample obtained from a subject having cancer or suspected to have cancer, a database of cancer patients, a database of cancer cell lines, or combinations thereof.

In some embodiments, the essentiality profile of the genes is determined based on the level of lethality of cells following the inhibition of expression or activity of the genes in the cells.

In some embodiments, the expression profile of the genes comprises a transcriptomic profile or a protein abundance profile of the cells.

In some embodiments, the processing circuitry, may be further configured to analyze the pair of genes to determine a score related to the association of said pair of genes.

In some embodiments, the processing circuitry may be further configured to generate an SL-network, based on the pairs of genes identified to interact via SL-interaction and/or on the strength of the SL-interaction between each pair.

In some embodiments, the processing circuitry may further be configured to determine an occurrence selected from the group consisting of:
i. response of cancer cells to the inhibition of a gene product;
ii. survival of a subject having cancer;
iii. response of cancer cells to a specific drug; and
iv. ranking of cancer treatments for a specific subject having cancer;
   by applying the identified SL-network on a genomic profile of cells, wherein the genomic profile of cells.

In some embodiments, the genomic profile of the cells may be obtained from a subject, a population of subjects, a genomic dataset, cancer cells of at least one subject, or any combination thereof.

In some embodiments, the survival of the subject having cancer is inversely-correlated to the number of the SL-paired genes which are co-inactive in the subject's tumor based on the determined SL-network and the genomic profile of the subject's tumor. In some embodiments, the presence of co-underexpressed SL-paired genes in the subject correlates with improved prognosis of survival of the subject having cancer compared to other subjects afflicted with cancer.

In some embodiments, the prediction of response of cancer cells to the inhibition of a gene product is utilized using a supervised mode or an unsupervised mode.

In some embodiments, the systems disclosed herein may further be used in a method of repurposing an active ingredient for use in cancer therapy, the method comprising applying SL-network or SDL-network on a genomic profile of cells, to identify the known active ingredients as candidates for targeting an identified SL gene or SDL gene, for treating cancer.

According to further embodiments, there is provided a method of repurposing an active ingredient to use in cancer therapy, the method comprising applying SL-network or SDL-network on a genomic profile of cells, to identify the known active ingredients as candidates for targeting an identified SL gene or SDL gene;
wherein the SL-network is produced using a data-driven computational system, the computational system is configured to identify SL-interaction of gene pairs comprising a first gene (A) and a second gene (B) by applying one or more of the following procedures:
   examine if an occurrence of co-inactivation in the cancer cells of the first gene and the second gene is lower than a predetermined threshold;
   determine if the essentiality of the second gene (B) is higher in the cancer cells in which the first gene (A) is inactive; and/or
   determine if the expression of the first gene and the second gene correlate with cancer;
   and;
   determine, based on said score, if the pair of genes interact via an SL-interaction, and to produce the SL-network based on the pairs of genes determined to have SL-interaction; or
wherein the SDL-network is produced using a data-driven computational system, the computational system is configured to identify SL-interaction of gene pairs comprising a first gene (A) and a second gene (B) by applying one or more of the following procedures:
   examine if an occurrence of over activation in the cancer cells of the first gene and inactivation of the second gene is lower than a predetermined threshold;
   determine if the essentiality of the second gene (B) is higher in the cancer cells in which the first gene (A) is overactive; and/or
   determine if the expression of the first gene and the second gene correlate with cancer;
   and;
   determine, based on said score, if the pair of genes interact via an SDL-interaction; and to produce the SDL-network based on the pairs of genes determined to have SDL-interaction.

In some embodiments, an active ingredient is a known active ingredient. In some embodiments, the known active ingredient to be repurposed for use in cancer therapy is selected from the group consisting of: Pentolinium, Imipramine, Dalfampridine, Amitriptyline, Verapamil and Dronedarone.

In some embodiments, the known active ingredient to be repurposed for used in cancer therapy may be used for treatment of subjects having VHL-deficient cancer. In some embodiments, the VHL-deficient cancer is VHL-deficient renal cancer.

In some embodiments, there is provided a method of treating cancer comprising administering to a subject in need thereof, a pharmaceutical composition comprising at least one active ingredient identified by the methods disclosed herein (i.e. identified to be repurposed for treating cancer). In some embodiments, the pharmaceutical composition comprises at least one active ingredient selected from the group consisting of: Pentolinium, Imipramine, Dalfampridine, Amitriptyline, Verapamil and Dronedarone. In some embodiments, the cancer is VHL-deficient

In some embodiments, there is provided a method of treating cancer comprising administering to a subject in need thereof a pharmaceutical composition comprising at least one active ingredient identified as a candidate for targeting an identified SL gene or SDL gene. In some embodiments, the at least one active ingredient is selected from the group consisting of: Pentolinium, Imipramine, Dalfampridine, Amitriptyline, Verapamil and Dronedarone.

In some embodiments, the present invention provides a method of predicting one or more occurrences selected from the group consisting of:
i. the response of cancer cells to the inhibition of a gene product;
ii. the survival of a subject having cancer;
iii. the response of cancer cells to a specific drug; and
iv. the ranking of cancer treatments for a specific subject having cancer;

the method comprising applying a Synthetic Lethal (SL) or a Synthetic Dosage Lethal (SDL) network on a genomic profile of cells.

According to some embodiments, the genomic profile is obtained from a subject, a population of subjects or a genomic dataset.

According to some embodiments, the genomic profile is obtained from cancer cells of at least one subject.

According to some embodiments, the survival of a subject having cancer (occurrence ii) is inversely-correlated to the number of SL-paired genes which are co-inactive in the patient's tumor according to the given SL-network and the genomic profile of the patient's tumor.

According to some embodiments the presence of co-underexpressed SL-paired genes in (ii), indicates better prognosis compared to other patients.

The present invention provides according to one aspect, a method of identifying Synthetic Lethal (SL) and and/or Synthetic Dosage Lethal (SDL)-interactions, and based upon, generating SL and SDL networks, using a direct data-driven computational system, wherein the computational system may utilize three types of profiles:
A gene-activity-profile, denoting the activity level of genes in a given cancer sample or cell line, according to the analysis of one or more of the following data types: Somatic Copy Number of Alterations (SCNA), germline Copy-Number Variations (CNV), DNA methylation, histone methylation, somatic or germline mutations; optionally, the gene-activity-profile can be further refined by accounting for the gene-expression-profile(s) (as described below), of the cancer sample or cell line;
A gene-essentiality-profile, denoting the level of lethality measured following the inhibition of various genes in a given cancer sample or cell line; gene inhibition can be obtained via, for example, shRNA, siRNA, mutagenesis, or drug administration;
A gene-expression-profile, denoting either a transcriptomic profile or a protein abundance profile of a given cancer sample or cell line.

In some embodiments, the computational system identifies SL-pairs by applying one or more of the following statistical inference procedures for every pair of genes (denoted as exemplary gene A and gene B):
I. “genomic Survival of the Fittest” (SoF) examines if the co-inactivation of both genes (A and B) occurs significantly less than expected by analyzing gene-activity-profiles.
II. “inhibition-based functional examination” integrates the gene-activity-profiles of a set of cancer samples with the gene-essentiality-profiles of these samples, and examines if gene B is significantly more essential in samples in which gene A is inactive.
III. “pairwise gene co-expression”, examines if the expression of genes A and B is correlated, by analyzing gene-expression-profiles.

In some embodiments, the computational system identifies SDL-pairs by applying the statistical inference procedure described above (III) as well as the following two procedures for every pair of genes (gene A and gene B):
I. “genomic Survival of the Fittest” (SoF) examines if the over-activation of gene A along with the inactivation of gene B occurs significantly less than expected by analyzing gene-activity-profiles.
II. “inhibition-based functional examination” integrates the gene-activity-profiles of a set of cancer samples with the gene-essentiality-profiles of these samples, and examines if gene B is significantly more essential in samples in which gene A is overactive.

For each gene-pair, five p-values are obtained according to each one of the statistical inference procedures described above. The p-values obtained in (I)-(III) denote the significance of the SL-interaction between the two genes, while the p-values obtained in (III)-(V) denote the significance of the SDL-interaction between the two genes. Gene-pairs with significantly low p-values (e.g., <0.01 following multiple hypotheses correction) are considered as predicted SL- or SDL-pairs.

According to some embodiments, the SL-network is identified using a data-driven computational system, wherein the computational system identifies SL-pairs by applying one or more of the following procedures for a given pair of genes (denoted as gene A and gene B):
I. “SL: genomic Survival of the Fittest (SoF)” examines if in cancer the co-inactivation of both genes (A and B) occurs significantly less than expected;
II. “SL: inhibition-based functional examination” examines if gene B is significantly more essential in cancer cells in which gene A is inactive;
III. “pairwise gene co-expression”, examines if the expression of genes A and B is correlated in cancer;
wherein the strength of the observed associations between gene A and gene B as described in I-III, above, is used to conclude whether the genes are interacting via an SL-interaction, and the strength of the interaction.

According to other embodiments, the SDL-network is identified using a data-driven computational system, wherein the computational system identifies SDL-pairs by applying one or more of the following procedures for a given pair of genes (denoted as gene A and gene B):
I. “SDL: genomic Survival of the Fittest” (SoF) examines if in cancer the over-activation of gene A along with the inactivation of gene B occurs significantly less than expected;
II. “SDL: inhibition-based functional examination” examines if gene B is significantly more essential in cancer cells in which gene A is overactive;
III. “pairwise gene co-expression”, examines if the expression of genes A and B is correlated in cancer;
wherein the strength of the observed associations between gene A and gene B as described in I-III, above, is used to conclude whether the genes are interacting via an SDL interactions, and the strength of the interaction.

According to some embodiments, the method comprises one or more of:
I. creating and initializing the following graphs: SoF_{SL}, SoF_{SDL}, functional_{SL}, functional_{SDL}, expression_{SL}, and expression_{SDL}, wherein SoF_{SL} and SoF_{SDL} are the SL and SDL networks constructed from SoF_{data}, respectively; functional_{SL} and functional_{SDL} are the SL and SDL networks constructed from functional_{data}, respectively; expression_{SL} and expression_{SDL} are the SL and SDL networks constructed from the expression_{data}, respectively;
II. input description: In the following description a genetic profile denotes a profile that consists of one or more of the following data: Somatic Copy Number of Alterations (SCNA), germline Copy-Number Variations (CNV), DNA methylation, histone methylation, somatic or germline mutations; an expression profile denotes either a transcriptomic profile or a protein abundance profile. Given a set of genes whose SL and SDL-partners are to be found (termed GeneList), and three sets of data:
   a. SoF_{datasets} referring to datasets that will be utilized to generate the SoF_{SL} and SoF_{SDL}, each dataset will include genomic profiles of a set of cancer samples, and optionally also the expression profiles of these samples;
   b. functional_{datasets} referring to dataset that will be utilized to generate the functional_{SL} and functional_{SDL}; each dataset will include the gene essentiality measurements taken from a cohort of cancer cell lines, along with the genomic profiles of these cell lines, and optionally also the expression profiles of these cell lines. Gene essentiality measurements can be obtained via shRNA, siRNA, or molecular inhibitors;
   c. expression_{datasets} referring to dataset that will be utilized to generate the expression_{SL} and expression_{SDL}; each dataset will include expression profiles of a set of clinical cancer samples or cancer cell lines;
III. for each pair of genes (A,B) ∈ [GeneList×GeneList]:
   a. determining whether (A,B) is to be added to SoF_{SL}:
   for every dataset I ∈ SoF_{datasets}
i. test via a statistical test (e.g., one-sided Wilcoxon rank-sum test) whether, in dataset I, gene B has higher SCNA levels in samples in which gene A is inactive compared to the rest of the samples; gene inactivation is deduced from the genomic and optionally also from the expression profiles of the samples in dataset I;
ii. let SL_SoF_{pvalue,I}(A,B) be the obtained p-value;
iii. if SL_SoF_{pvalue,I}(A,B) following Bonferroni correction is below 0.05 add (A,B) to SoF_{SL};
   b. determining whether (A,B) is to be added to SoF_{SDL}:
   for every dataset I ∈ SoF_{datasets}
i. test via a statistical test (e.g., one-sided Wilcoxon rank-sum test) whether, in dataset I, gene B has higher SCNA levels in samples in which gene A is overactive compared to the rest of the samples; gene overactivation is deduced from the genomic and optionally also from the expression profiles of the samples in dataset I;
ii. let SDL_SoF_{pvalue,I}(A,B) be the obtained p-value;
iii. if SDL_SoF_{pvalue,I}(A,B) following Bonferroni correction is below 0.05 add (A,B) to SoF_{SDL};
   c. determining whether (A,B) is to be added to functional_{SL}:
   for every dataset I ∈ functional_{datasets}
i. test via a statistical test (e.g., one-sided Wilcoxon rank sum test) whether, in dataset I, the inhibition of gene B is more lethal in samples in which gene A is inactive compared to the rest of the samples. gene inactivation is deduced from the genomic and optionally also from the expression profiles of the samples in dataset I;
ii. let SL_functional_{pvalue,I}(A,B) be the obtained p-value;
iii. if SL_functional_{pvalue,I}(A,B)<0.05 add (A,B) to functional_{SL};
   d. determining whether (A,B) is to be added to functional_{SDL}:
   for every dataset I ∈ functional_{datasets}
i. Test via a statistical test (e.g., one-sided Wilcoxon rank sum test) whether, in dataset I, the inhibition of gene B is more lethal in samples in which gene A is overactive compared to the rest of the samples; gene overactivation is deduced from the genomic and optionally also from the expression profiles of the samples in dataset I;
ii. Let SDL_functional_{pvalue,I}(A,B) be the obtained p-value;
iii. If SDL_functional_{pvalue,I}(A,B)<0.05 add (A,B) to functional_{SDL};
   e. determining whether (A,B) is to be added to mRNA_{SL} and mRNA_{SDL}:
   for every dataset I ∈ expression_{datasets}
i. compute the Spearman correlation between the expression of gene A and gene B in dataset I;
ii. let expression_{pvalue,I}(A,B) be the correlation p-value, and expression_{correlation,I}(A,B) be the correlation coefficient;
iii. if expression_{correlation,I}(A,B)≧Rₘᵢₙ, and expression_{pvalue,I}(A,B) following Bonferroni correction is below 0.05 add (A,B) to expression_{SL} and to expression_{SDL};
IV.
   a. creating an SL output network as the intersection of networks SoF_{SL}, functional_{SL}, and expression_{SL}, such that an edge exists in the combined graph only if it appears in the three graphs;
   b. creating an SDL output network as the intersection of graphs SoF_{SDL}, functional_{SDL}, and expression_{SDL}, such that an edge exists in the combined graph only if it appears in the three graphs;
V. for every inference procedure combine the p-values obtained by its datasets into a single p-value per gene-pair via Fisher's combined probability test:
   a. SL_SoFₚᵥₐₗᵤₑ(A,B)=Fisher's_Method({SL_SoF_{pvalue,I}(A,B)|I∈ SoF_{datasets}})
   b. SL_functionalₚᵥₐₗᵤₑ(A,B)=Fisher's_Method({SL_functional_{pvalue,I}(A,B)|I∈functional_{datasets}})
   c. SDL_SoFₚᵥₐₗᵤₑ(A,B)=Fisher's_Method({SDL_SoF_{pvalue,I}(A,B)|I∈ SoF_{datasets}})
   d. SDL_functionalₚᵥₐₗᵤₑ(A,B)=Fisher's_Method({SDL_functional_{pvalue,I}(A,B)|I∈functional_{datasets}})
   e. expressionₚᵥₐₗᵤₑ(A,B)=Fisher's_Method({expression_{pvalue,I}(A,B)|I∈expression_{datasets}})
VI. further integrated the three combined p-values into one p-value per gene-pair, again via Fisher's method, considering all inference procedures:
   SL_Allₚᵥₐₗᵤₑ(A,B)=Fisher's_Method(SL_SoFₚᵥₐₗᵤₑ(A,B)∪SL_functionalₚᵥₐₗᵤₑ(A,B)∪expressionₚᵥₐₗᵤₑ(A,B)})
   SDL_Allₚᵥₐₗᵤₑ(A,B)=Fisher's_Method(SDL_SoFₚᵥₐₗᵤₑ(A,B)∪SDL_functionalₚᵥₐₗᵤₑ(A,B)∪expressionₚᵥₐₗᵤₑ(A,B)})
VII. for each pair of genes (A,B) ∈ [GeneList×GeneList] return SL_SoFₚᵥₐₗᵤₑ(A,B), SDL_SoFₚᵥₐₗᵤₑ(A,B), SL_functionalₚᵥₐₗᵤₑ(A,B), SDL_functionalₚᵥₐₗᵤₑ(A,B), expressionₚᵥₐₗᵤₑ(A,B), and SL_Allₚᵥₐₗᵤₑ(A,B), SDL_Allₚᵥₐₗᵤₑ(A,B).

The present invention provides according to one aspect, a method of applying SL and SDL networks for predicting the response of cancer cells to the inhibition of a gene product, based on the genomic profile of the cells. In some embodiments, the genomic profile of the cells can be a profile of SCNA, mutations, DNA or histone methylation, gene expression (mRNA) or protein abundance.

According to some embodiments, the method is utilized in an unsupervised mode wherein, 1) for each sample, inactive and overactive genes are identified according to its genomic profile; and 2) the viability of a given sample is predicted following the inhibition of a given gene as proportional to the number of inactive SL-partners and overactive SDL-partners the pertaining gene has in the given sample.

According to other embodiments, the method is utilized in a supervised mode wherein, important features of the network and relevant genetic characteristics of the tumor are extracted and utilized to train and utilize machine learning predictors. The training of the predictors is done according to some embodiments by integrating experimental measurements of gene essentiality or drug efficacy. The machine learning predictors according to some embodiments are Support Vector Machine (SVM) classifiers or Neural Network predictors.

In some embodiments, an SL and/or SDL networks produced by the above method is also within the scope of the present invention as well as its uses.

According to some embodiments, the SL network comprises the gene pairs presented in Table 1.

According to other embodiments, the SDL network comprises the gene pairs presented in Table 2.

According to some embodiments the SL/SDL network comprises the gene pairs presented in Tables 1 and 2.

According to some embodiments, the genomic data is selected from the group consisting of: Somatic copy Number of Alterations (SCNA), germline copy number variations, somatic or germline mutations, gene expression (mRNA levels), protein abundance, DNA or histone methylation.

According to other embodiments, the genomic data is obtained from a source selected from the group consisting of: a sample taken from a subject having cancer or suspected to have cancer, a database of cancer patients, a database of cancer cell lines.

According to some embodiments the method is used to predict cancer gene essentiality and thus to provide potential targets for cancer therapy in an individual in need of such treatment or in a population or sub-population of cancer patients.

According to other embodiments, the method is used to assess prognosis for a subject having cancer.

According to another aspect, the invention provides a method of predicting survival of a subject having cancer based on the genomic profile of its cancer cells; the patient survival is inversely-correlated to the number of SL-paired genes which are co-inactive in the patient's tumor according to the given SL-network and the genomic profile of the patient's tumor.

Another aspect of the present invention relates to a method of providing a personalized cancer treatment comprising utilization of the DAISY system (approach) for identifying the optimal treatment in a specific patient or in a sub-population of patients having cancer.

According to some embodiments, specific anti-cancer therapy is provided based on the existence of specific SL/SDL-interactions.

According to another aspect, a method of predicting drug responses is provided comprising utilizing the DAISY system by analyzing the genomic data obtained from a subject, a population of subjects or a genomic dataset.

According to yet another aspect, the system and methods of the present invention provide repurposing known active ingredients for cancer therapy.

According to some embodiments the active ingredients are selected from the group consisting of: Pentolinium, Imipramine, Dalfampridine, Amitriptyline, Verapamil and Dronedarone.

The system and methods of the present invention are also used for identification of new drug targets for treating cancer.

According to some embodiments, the drug targets are selected from the genes listed in Table 3.

According to another embodiment, a drug target for treating cancer is provided and may be selected from the genes listed in Table 4.

According to another embodiment, a drug target for treating cancer is provided and may be selected from the genes listed in Table 5.

According to yet another aspect, a method of treating cancer is provided comprising administering to a subject in need thereof, a pharmaceutical composition comprising at least one agent that target a gene which was identified as part of an SL/SDL pair by a method according to the present invention.

According to some embodiments, the pharmaceutical composition comprises at least one agent selected from the group consisting of: Pentolinium, Imipramine, Dalfampridine, Amitriptyline, Verapamil and Dronedarone.

According to some embodiments, the drug targets are selected from the genes listed in Table 3.

According to another embodiment, a drug target for treating cancer is provided selected from the genes listed in Table 4.

According to some specific embodiments SL-based treatment according to the present invention induces the reactivation of a tumor suppressor or the inactivation of an oncogene by targeting its SL- or SDL-pair, respectively.

Furthermore, a method of predicting the likelihood that a patient's cancer will respond to a specific therapy is provided. According to some embodiments of this aspect, a sample of cells taken from a biopsy or from a surgical removal of a tumor in a subject having cancer, is determined for the expression level of specific genes or somatic copy of alterations, and the resulted data is integrated with an SL/SDL network of the present invention using an unsupervised or a supervised approach.

According to some embodiments, the response of a tumor to inhibitors of a molecule selected from the group consisting of: EGFR, PARP1, BCL2, and HDAC2 is predicted using an SDL-network according to the present invention.

According to a specific embodiment, the SDL network comprises the gene-pairs listed in Table 3.

Also provided is a method for ranking specific cancer treatments for a patient in need by integrating the SL/SDL-network with the genomic characteristics of the patient's tumor.

According to some specific embodiments the subject tumor is not a tumor characterized by overactivation or inactivation of cancer associated genes such as onco-genes or tumor suppressors.

According to other embodiments the system and methods of the present invention are used for targeting genetically unstable tumors that harbor many partial gene deletions and amplifications.

In yet another aspect, methods of identifying SL/SDL-networks of specific cancer types are provided, comprising utilizing DAISY for analysis of molecular datasets of specific cancer types.

According to some embodiments, the methods of the present invention comprise integration of additional types of data, including methylation data.

According to some embodiments, SL-based therapy further help in counteracting resistance to treatment, when targeting a gene that was identified by the methods of the present invention to lose a high number of SL-partners.

According to some embodiments, SL-based therapy may further aid in counteracting resistance to treatment, when targeting a gene whose inactive SL-partners and overactive SDL-partners reside on different chromosomes or in distant genomic locations.

According to another aspect, the invention provides a method of predicting survival of a subject having cancer comprising analyzing cells taken from a tumor of the subject by the methods described above and identifying SL-paired genes, wherein the presence of underexpressed SL-paired genes indicates better prognosis compared to other patients.

According to some embodiments, the cancer is breast cancer.

According to some embodiments, the SL-paired genes are selected from the pairs listed in Tables 1 and 4-5.

According to some embodiments, there is provided a method of treating cancer comprising administering to a patient in need thereof, a drug combination comprising an agent which target X and an agent that target Y, where X and Y represent an SL-pair identified by DAISY, according to the present invention.

According to some embodiments, the therapeutic and prognostic applications described in the present invention are relevant to any cancer of a mammalian, preferably a human subject.

According to some embodiments, the cancer is a metastatic cancer.

According to other embodiments, the cancer is a solid cancer.

According to yet another aspect, the present invention provides a method of preventing or treating tumor metastasis comprising administering to a subject in need thereof a pharmaceutical composition comprising at least one agent disclosed above or identified by a method disclosed above.

According to some embodiments the metastasis is decreased. According to other embodiments, the metastasis is prevented. According to yet other embodiments, the spread of tumors to the lungs of said subject is inhibited.

Pharmaceutical composition comprising active agent according to the present invention may be administered as a stand-alone treatment or in combination with a treatment with any anti-neoplastic agent.

According to a specific embodiment, the anti-neoplastic composition comprises at least one chemotherapeutic agent. The chemotherapeutic agent, which could be administered separately or together with an agent according to the present invention, may comprise any such agent known in the art exhibiting anti-cancer activity, including but not limited to: mitoxantrone, topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, and megestrol acetate. According to a specific embodiment, the chemotherapeutic agent is selected from the group consisting of alkylating agents, antimetabolites, folic acid analogs, pyrimidine analogs, purine analogs and related inhibitors, vinca alkaloids, epipodopyllotoxins, antibiotics, L-asparaginase, topoisomerase inhibitor, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. According to another embodiment, the chemotherapeutic agent is selected from the group consisting of 5-fluorouracil (5-FU), leucovorin (LV), irenotecan, oxaliplatin, capecitabine, paclitaxel and doxetaxel. Two or more chemotherapeutic agents can be used in a cocktail to be administered in combination with administration of the antibody or fragment thereof.

According to a specific embodiment, the invention provides a method of treating cancer in a subject, comprising administering to the subject effective amount of an active agent identified by any of the methods of the present invention.

The cancer amendable for treatment by the present invention includes, but is not limited to: carcinoma, lymphoma, blastoma, sarcoma, and leukemia or lymphoid malignancies. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic NHL; intermediate grade/follicular NHL; intermediate grade diffuse NHL; high-grade immunoblastic NHL; high-grade lymphoblastic NHL; high-grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of breast cancer, colorectal cancer, rectal cancer, non-small cell lung cancer, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. The cancerous conditions amendable for treatment of the invention include metastatic cancers.

In another aspect, the present invention provides a method for increasing the duration of survival of a subject having cancer, comprising administering to the subject effective amount of a composition comprising an active agent identified by the present invention.

In yet another aspect, the present invention provides a method for increasing the progression free survival of a subject having cancer, comprising administering to the subject effective amount of a composition comprising an active agent identified by any of the methods of the present invention.

Furthermore, the present invention provides a method for treating a subject having cancer, comprising administering to the subject effective amounts of a composition comprising an active agent identified by any of the methods of the present invention.

In yet another aspect, the present invention provides a method for increasing the duration of response of a subject having cancer, comprising administering to the subject effective amount of a composition comprising an active agent identified by any of the methods of the present invention.

In another aspect, the invention provides a method of preventing or inhibiting development of metastasis in a patient having cancer, comprising administering to the subject effective amounts of a composition comprising an active agent identified by any of the methods of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Exemplary embodiments are illustrated in referenced figures. Dimensions of components and features shown in the figures are generally chosen for convenience and clarity of presentation and are not necessarily shown to scale. The figures are listed below.

FIG. 1 demonstrates the concept of graph and graph intersection, in accordance with some embodiments of the disclosure;

FIG. 2 shows an exemplary system for creating and manipulating graphs according to the invention. A computing platform 200, comprising one or more processors 204, any of which may be any Central Processing Unit (CPU), a microprocessor, an electronic circuit, an Integrated Circuit (IC) or the like. Alternatively, processor 204 can be implemented as hardware or configurable hardware such as field programmable gate array (FPGA) or application specific integrated circuit (ASIC). Processor 204 can be implemented as firmware written for or ported to a specific processor such as digital signal processor (DSP) or microcontrollers. Processor 204 may be used for performing mathematical, logical or any other instructions required by computing platform 200 or any of it subcomponents.

FIG. 3 shows a diagram illustrating the DAISY workflow. The three different inference procedures described in the main text are applied in parallel to identify SL or SDL gene-pairs. The SL/SDL-networks are then assembled from gene-pairs that are identified in all three procedures (colored intersection).

FIGS. 4A, 4B and 4C show graphs demonstrating that DAISY-inferred SL- and SDL-interactions match experimentally detected interactions in cancer. FIG. 4A: The overall ROC-curves obtained when predicting SL-interactions of major cancer genes including MSH2, PARP1 and VHL, and SDL-interactions involving KRAS. The ROC-curves show the performances obtained when predicting SDL/SLs by analyzing each of the three data types separately—SCNA, mRNA, and shRNA—using both SCNA and mRNA datasets (Combined (SCNA+mRNA), and finally, based on all datasets (Combined). The black diagonal line denotes the random, theoretical ROC-curve as a control. FIG. 4B: The SCNA and expression patterns of experimentally well-established SL-pairs PARP1-BRCA1. FIG. 4C: The SCNA and expression patterns of experimentally well-established SL-pairs PARP1-BRCA2. For each one of these SL-pairs the SCNA levels of one gene are significantly higher when its partner is deleted than when its partner is retained (one-sided Wilcoxon rank sum test).

FIG. 5 shows bar-graphs of assays examining DAISY predictions of VHL-SLs. The mean percentage of growth inhibition of VHL-deficient and VHL-restored cell lines at the mid-effective concentration of each drug. All the drugs besides Staurosporine (positive control) were predicted to selectively inhibit the growth of VHL-deficient cells. On top of the bars are the one-sided t-test p-values obtained when examining if the inhibition of the VHL-deficient cells is higher than the inhibition of VHL-restored cells.

FIGS. 6A, 6B and 6C show graphs of assays for predicting cell-specific gene essentiality based on the SL-network. FIGS. 6A-B: The experimental essentiality scores of genes across different cancer cell lines as a function of the number of SL-partners they have lost, according to (FIG. 6A) the Marcotte, and (FIG. 6B) Achilles screens (lower experimental gene essentiality scores denote higher essentiality). FIG. 6C: The ROC curves obtained when using the SL-based neural network predictors to predict gene essentiality in BT549, and testing the predictions according to the refined set of genes that were found as essential across all three BT549 screens. The predictors were trained based on the gene essentiality of the Marcotte and Achilles screens, excluding the BT549 cell line data that was used exclusively for testing.

FIGS. 7A and 7B show graphs predicting clinical prognosis based on the SL-network. In parenthesis next to name of each group are the number of patients, and the number and percentage of deaths in that group. FIG. 7A: The KM-plot obtained when dividing the breast cancer samples according to the expression of POLA2 and KIF14 (the most predictive SL-pair in terms of breast cancer prognosis). The arrows point to the estimated effect of KIF14 underexpression, in the context of POLA2 expression and underexpression, respectively (the legend refers to the curves in their order, from top to bottom). FIG. 7B: KM-plots depicting the survival of samples that co-underexpressed a high number of SL-pairs (global SL-score above the 90th percentile, upper curve), and of samples that co-underexpressed a low number of SL-pairs (global SL-score below the 10th percentile, lower curve).

FIGS. 8A, 8B and 8C show graphs demonstrating that the SDL-network predicts the efficacy of anticancer drugs in cancer cell lines. FIG. 8A: The IC50s (left) and area-under-does-curve (right) of drugs decrease in cell lines where their target(s) have an increasing number of overexpressed SDL-partners (lower values denote higher efficacy). FIGS. 8B-C show the drug efficacy predictions obtained by a supervised neural network predictor based on SDL-features: FIG. 8B—the predicted vs. experimental IC50 log values of 41 drugs measured across 414 cancer cell lines (CGP data); FIG. 8C—the predicted vs. experimental area-under-dose-curve of 50 drugs measured across 241 cancer cell lines (CTRP data).

### DETAILED DESCRIPTION OF THE INVENTION

According to some embodiments, the systems and methods disclosed herein for identification of Synthetic Lethal (SL)-interactions and networks and/or Synthetic dosage Lethal (SDL)-interactions and networks and uses thereof allow for the first time the data driven identification of cancer Synthetic-lethality in a genome-wide manner.

According to some embodiments, the system and methods disclosed herein provide the first approach enabling a data driven identification of cancer Synthetic-lethality in a genome-wide manner. The approach, termed herein DAta-mIning SYnthetic-lethality-identification pipeline (DAISY) successfully captures the results obtained in key large-scale experimental studies exploring SLs in cancer. For the first time, it enables the prediction of gene essentiality, drug efficacy, and/or clinical prognosis stemming from SL/SDL interactions in cancer.

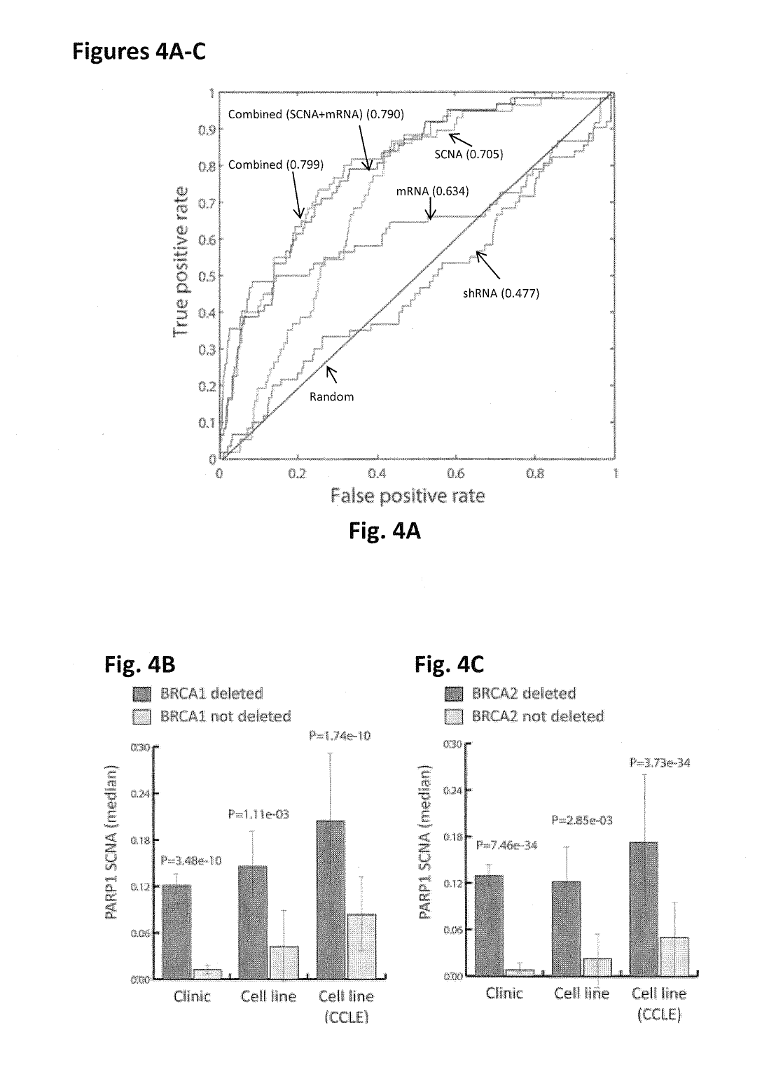

DAISY presents a complementary effort to current genetic and chemical screens, narrowing down the number of gene-pairs that need to be examined experimentally to detect SL and SDL interactions in cancer. For example, based on the true positive and false positive rates presented in FIG. 4A, one can compute how much experimental work can be saved by starting off from the provided predictions, instead of searching the whole combinatorial space of interactions. Accordingly, an experimental screen for discovering SL-interactions could be designed to check the SL-pairs predicted by DAISY such that 5%, 25%, 50% or 70% of all the SL-interactions that are out there will be detected by examining only 0.25%, 4%, 14%, or 24% of all possible gene-pairs, respectively. That is, testing only the top (most confident) 0.25% of the SLs predicted will enable to find 5% of all SL-interactions, thus detecting up to 20 times more SL-pairs than expected by random. Likewise, it is demonstrated that by applying DAISY to design a screen for detecting the SL-interactions of VHL it is possible to detect almost four times as many SL-interactions compared to a screen that was designed by applying a biological reasoning. Hence, DAISY could facilitate a more rapid and rational discovery of SL-interactions in cancer by guiding focused experimental screens.

In some embodiments, SL-networks that include interactions shared by different types of cancers were generated and are disclosed herein. In some embodiments, application of DAISY for the analysis of these emerging datasets may be further used to identify SL and SDL networks of specific cancer types. Furthermore, the additive nature of DAISY enables its straightforward refinement with the integration of additional types of data. Likely, such data may include methylation data, and the integration of somatic mutations to detect SDL interactions, when reliable algorithms for identifying over-activating mutations are used. This additional information could be used both to better identify SL-interactions via DAISY, and also to better identify over-active and inactive genes when employing the networks to predict essentiality, drug response and survival.

Complete gene loss is a rather infrequent event. Hence, to construct and utilize the SL-network, gene inactivation thresholds were defined permissively, based on gene copy-number and expression. However, as implied by the results provided herein, in many cases such a partial inactivation of a gene still suffices to induce the essentiality of its SL-partners. More importantly, it is shown that SL and SDL interactions have a marked cumulative effect. These results suggest that a gene can form a useful drug target due to the partial inactivation or overactivation of several of its SL or SDL-partners, respectively. SL-based treatment is therefore a promising avenue especially for targeting genetically unstable tumors that harbor many partial gene deletions and amplifications. The presence of several inactive SL (and/or overactive SDL) partners in a given tumor may enable a drug to kill a broad array of genomically heterogeneous cells, each sensitive to the drug due to the inactivity of a different subset of the SL-partners and/or over-activity of the SDL-partners of its targets. Targeting a gene that has a high number of inactive SL and/or overactive SDL-partners may further help in counteracting the daunting problem of emerging resistance to treatment, especially if its partners reside on different chromosomes or in distant genomic locations. Another important beneficial aspect of SL-based treatment is that it can induce the reactivation of a tumor suppressor or the inactivation of an oncogene by targeting its SL- or SDL-pair, respectively.

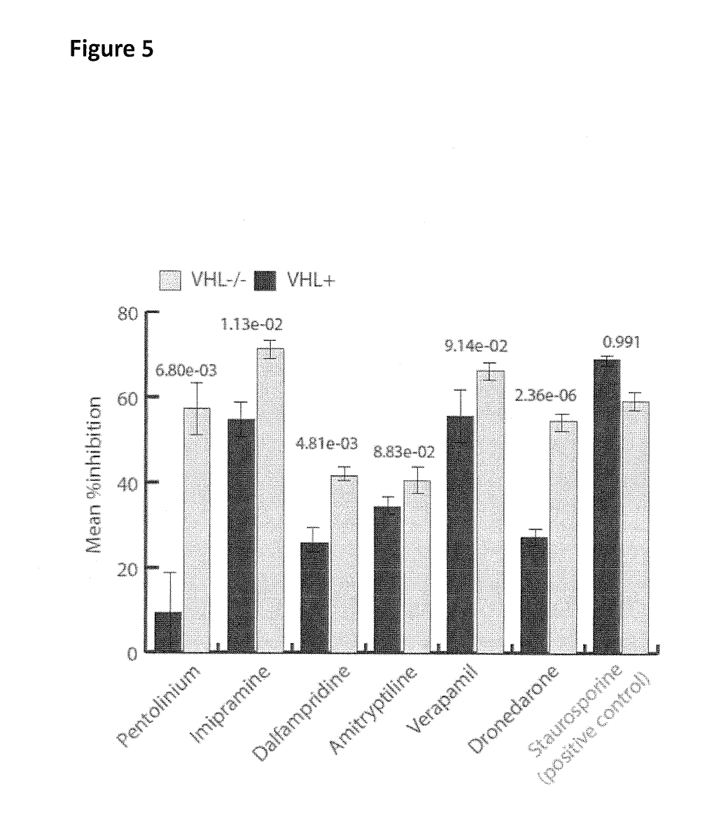

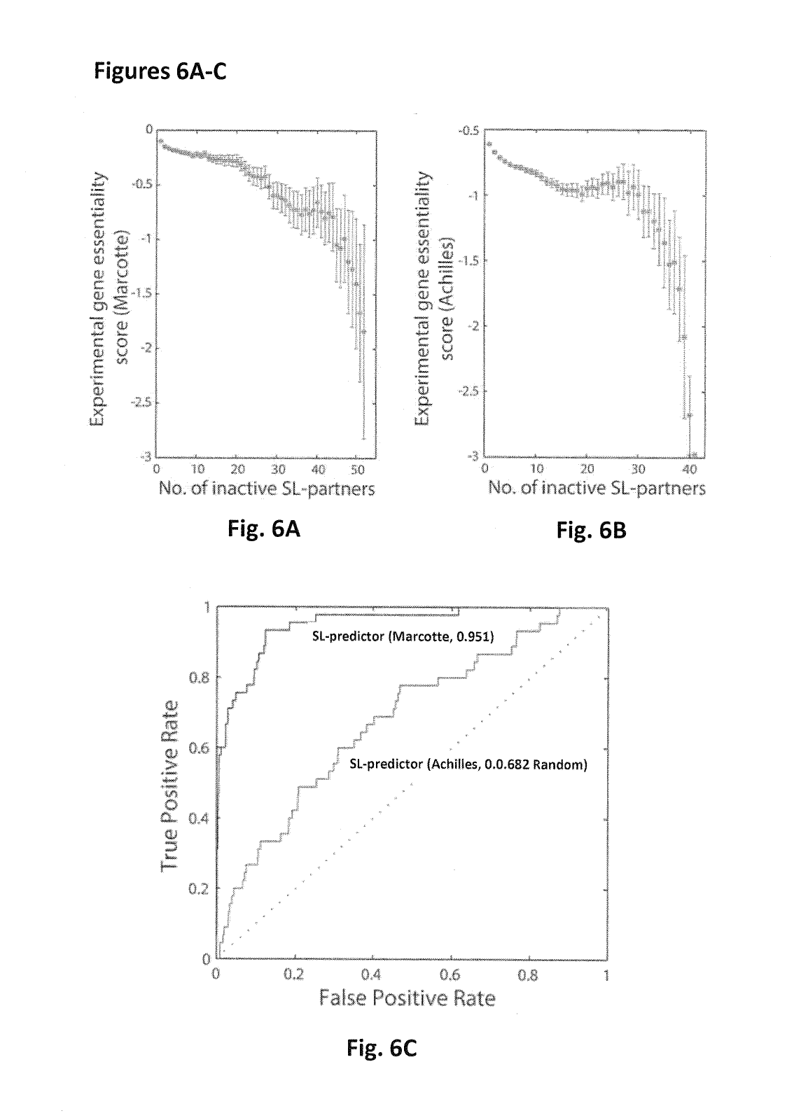

According to some embodiments, computational methods and systems, such as those provided herein, alongside focused experimental screens, are used for the generation of well-established genome-scale SL and SDL networks. Such networks can be applied in various ways to gain insights into the biology of the tumor, and identify its vulnerabilities in a personalized manner. More specifically, various challenges may be tackled by utilizing SL and/or SDL networks: (1) ranking existing treatments for a given patient, (2) repurposing drugs, (3) finding new drug targets, and (4) predicting patient prognosis. For example, for ranking existing treatments for a given patient (1), as demonstrated herein, an SDL-network can be utilized to predict the efficacy of approved anticancer drugs in a cell line specific manner. Likewise, SDL networks may provide a platform to rank anticancer drugs per patient based on the genomic characteristics of the tumor. For examples, for repurposing drugs (2), performing this task while considering not only anticancer drugs but also clinically approved drugs that are currently used to treat other diseases may contribute to the ongoing efforts of drug repurposing in cancer. As detailed herein, it was found that according to the SL-interactions predicted by systems and methods disclosed herein, tumors with VHL-deficiency are sensitive to drugs that are currently used for treating hypertension (Pentolinium, Verapamil), depression (Amitriptyline, Imipramine), and multiple sclerosis (Dalfampridine). As demonstrated below, it was found that VHL-deficient cells are significantly more sensitive to these drugs compared to isogenic cells in which pVHL was restored (FIG. 5). For example, for finding new drug targets (3) the SL-network was applied to predict gene essentiality in cancer cell lines. The same methodology can be applied to predict gene essentiality in clinical samples, leading to a systematic identification of new potential drug-targets. For example, as demonstrated herein, for predicting patient prognosis (4), such as cancer prognosis, SL-interactions may be used. As shown herein, breast cancer patients whose tumors co-underexpressed SL-paired genes had significantly better prognosis compared to other patients (FIG. 6). Taken together, SL and SDL-network-based analysis combined with personalized genomics can provide an important future tool for assessing response to treatment, and for tailoring more selective and effective personalized therapeutics.

### The Computational Aspect

In computer science, a graph is an abstract data type used for implementing the graph concept from mathematics. A graph may be implemented in a multiplicity of ways, using various data structures, data structure collections, linking mechanisms such as but not limited to pointers, or the like.

A graph generally comprises nodes (also referred to as vertices) and edges connecting two nodes. In many cases, each node represents an object and each edge represents a connection between object. In some cases, each edge may be associated with one or more properties, such as an identifier or quantifier associated with the connection between the objects, such as weight, significance or other properties. Edges may be directional or bidirectional.

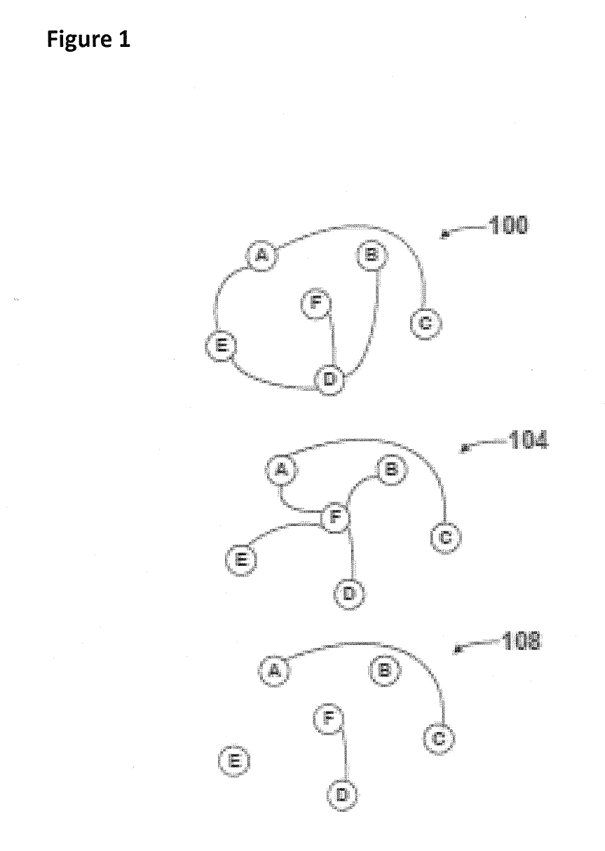

Referring now to FIG. 1, demonstrating a visual representation of a graph and the operation of graph intersection.

Graph 100 comprises six nodes, indicated A, B, C, D, E, and F. The nodes may represent any entity relevant for the problem to be solved, for example genes.

Graph 100 further comprises edges A-E, A-C, E-D, D-F and D-B, each representing a connection between the two nodes at its ends. For example, each node may represent that the two genes form a synthetic lethal (SL) pair, or a synthetic dosage lethal (SDL) pair.

Graph 104 comprises the same nodes, and edges A-F, F-C, F-B, F-E, F-D and A-C.

Graph 108 is the intersection graphs 100 and 104, since it comprises the same nodes, but only the edges appearing in the two graphs, i.e. edges A-C and F-D.

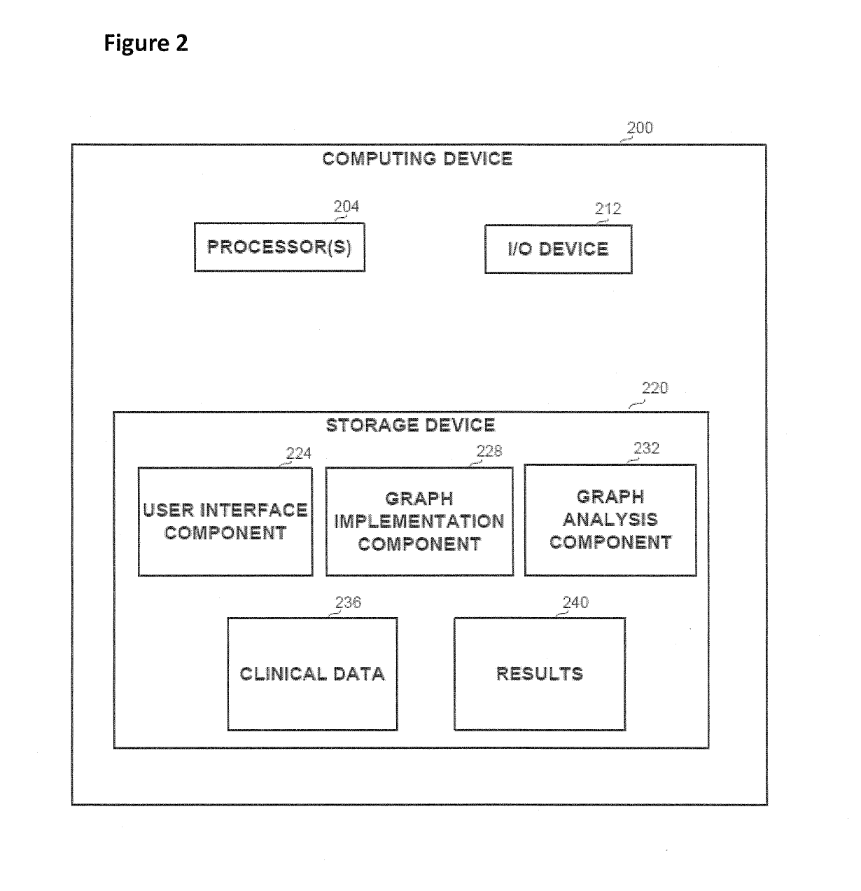

Referring now to FIG. 2, showing an exemplary system for creating and manipulating interactions and networks (graphs), according to some embodiments.

According to some embodiments, the system of the present invention may generally comprise a computing platform 200, comprising one or more processors 204, any of which may be any processing circuitry, such as Central Processing Unit (CPU), a microprocessor, an electronic circuit, an Integrated Circuit (IC) or the like. Processor 204 can be implemented as hardware or configurable hardware such as field programmable gate array (FPGA) or application specific integrated circuit (ASIC). In yet other alternatives, processor 204 can be implemented as firmware written for or ported to a specific processor such as digital signal processor (DSP) or microcontrollers. Processor 204 may be used for performing mathematical, logical or any other instructions required by computing platform 200 or any of it subcomponents.

In some embodiments, computing platform 200 may comprise an input/output device 212 such as a keyboard, a mouse, a touch screen, a display, or any other device used for receiving data or commands from a user, or displaying options or output to the user.

In some exemplary embodiments, computing platform 200 may comprise or be associated with one or more storage devices such as storage device 220. Storage device 220 may be non-transitory (non-volatile) or transitory (volatile). For example, storage device 220 can be a Flash disk, a Random Access Memory (RAM), a memory chip, an optical storage device such as a CD, a DVD, or a laser disk; a magnetic storage device such as a tape, a hard disk, storage area network (SAN), a network attached storage (NAS), or others; a semiconductor storage device such as Flash device, memory stick, or the like. Storage device 220 may contain user interface component 224 for receiving input or providing output to and from server 400 or a user.

Storage device 220 may further contain graph implementation component 228 for performing calculations for creating and manipulating graphs, for example intersecting graphs. Creating the graph may use calculations involving data from the available results.

Storage device 220 may further comprise graph analysis component 232 for analyzing the constructed graphs, and drawing conclusions, such as for identifying effective treatment for a patient, assessing effectiveness of a treatment of providing prognosis for a patient.

Storage device 220 may also store data such as clinical data 236 and results 240.

In some embodiments, interactions between genes may be described as a graph, also referred to as a network, in which each node represents a gene, and each edge represents the synergy level between the genes represented by its end nodes, for example each edge is associated with a p-value representing the strength of the interaction between the genes.

The input to creating the graph(s) is one or more datasets of genomic, molecular and/or clinical data, including, for example: SCNA, CNV, DNA methylation, histone methylation, somatic or germline mutations, transcriptomics, proteomics, and gene essentiality measurements obtained via shRNA, siRNA, mutagenesis, or drug administration, and the output is a collection of gene pairs and a weight associated with each pair. In some embodiments, the datasets may include activity profile of the genes, essentiality profile of the genes, expression profile of the genes, or combinations thereof.

In some embodiments, two graphs/networks may be generated: an SL graph (network), and/or an SDL graph (network).

In some embodiments, one or more statistical inference approaches may be used to assess the weight of each such pair in each graph, and the total weight may be assessed as a combination of the separate assessments.

A first inference approach (procedure) may be the genomic Survival of the Fittest (SoF) conducted by analyzing one or more of the following data, denoted as SoF-datasets: SCNA, CNV, DNA methylation, histone methylation, somatic or germline mutations profiles of cancer cell lines and clinical samples.

A second inference approach (procedure) may be the inhibition-based functional examination, conducted by analyzing the results obtained in gene essentiality (shRNA) screens together, with the SCNA and gene expression profiles of the cancer cell lines examined in the pertaining screen, denoted as functional-datasets.

A third inference approach (procedure) relates to pairwise gene co-expression, conducted by analyzing gene expression profiles, denoted as expression-datasets.

The approaches and their combination may be applied in methods of identifying Synthetic Lethal (SL) and Synthetic Dosage Lethal (SDL)-interactions, and generating SL and SDL networks, using a direct data-driven computational system:
I. creating and initializing the following graphs: SoF_{SL}, SoF_{SDL}, functional_{SL}, functional_{SDL}, expression_{SL}, and expression_{SDL}, wherein SoF_{SL} and SoF_{SDL} are the SL and SDL networks constructed from SoF_{data}, respectively; functional_{SL} and functional_{SDL} are the SL and SDL networks constructed from functional_{data}, respectively; expression_{SL} and expression_{SDL} are the SL and SDL networks constructed from the expression_{data}, respectively;
II. input description: In the following description a genetic profile denotes a profile that consists of one or more of the following data: Somatic Copy Number of Alterations (SCNA), germline Copy-Number Variations (CNV), DNA methylation, histone methylation, somatic or germline mutations; an expression profile denotes either a transcriptomic profile or a protein abundance profile. Given a set of genes whose SL and SDL-partners are to be found (termed GeneList), and three sets of data:
   a. SoF_{datasets} referring to datasets that will be utilized to generate the SoF_{SL} and SoF_{SDL}, each dataset will include genomic profiles of a set of cancer samples, and optionally also the expression profiles of these samples;
   b. functional_{datasets} referring to dataset that will be utilized to generate the functional_{SL} and functional_{SDL}; each dataset will include the gene essentiality measurements taken from a cohort of cancer cell lines, along with the genomic profiles of these cell lines, and optionally also the expression profiles of these cell lines. Gene essentiality measurements can be obtained via shRNA, siRNA, or molecular inhibitors;
   c. expression_{datasets} referring to dataset that will be utilized to generate the expression_{SL} and expression_{SDL}; each dataset will include expression profiles of a set of clinical cancer samples or cancer cell lines;
III. for each pair of genes (A,B) ∈ [GeneList×GeneList]:
   a. determining whether (A,B) is to be added to SoF_{SL}:
   for every dataset I ∈ SoF_{datasets}
   i. test via a statistical test (e.g., one-sided Wilcoxon rank sum test) whether, in dataset I, gene B has higher SCNA levels in samples in which gene A is inactive compared to the rest of the samples; gene inactivation is deduced from the genomic and optionally also from the expression profiles of the samples in dataset I;
   ii. let SL_SoF_{pvalue,I}(A,B) be the obtained p-value;
   iii. if SL_SoF_{pvalue,I}(A,B) following Bonferroni correction is below 0.05 add (A,B) to SoF_{SL};
   b. determining whether (A,B) is to be added to SoF_{SDL}:
      for every dataset I ∈ SoF_{datasets}
   i. test via a statistical test (e.g., one-sided Wilcoxon rank sum test) whether, in dataset I, gene B has higher SCNA levels in samples in which gene A is overactive compared to the rest of the samples; gene overactivation is deduced from the genomic and optionally also from the expression profiles of the samples in dataset I;
   ii. let SDL_SoF_{pvalue,I}(A,B) be the obtained p-value;
   iii. if SDL_SoF_{pvalue,I}(A,B) following Bonferroni correction is below 0.05 add (A,B) to SoF_{SDL};
   c. determining whether (A,B) is to be added to functional_{SL}:
      for every dataset I ∈ functional_{datasets}
   i. test via a statistical test (e.g., one-sided Wilcoxon rank sum test) whether, in dataset I, the inhibition of gene B is more lethal in samples in which gene A is inactive compared to the rest of the samples. gene inactivation is deduced from the genomic and optionally also from the expression profiles of the samples in dataset I;
   ii. let SL_functional_{pvalue,I}(A,B) be the obtained p-value;
   iii. if SL_functional_{pvalue,I}(A,B)<0.05 add (A,B) to functional_{SL};
   d. determining whether (A,B) is to be added to functional_{SDL}:
      for every dataset I ∈ functional_{datasets}
   i. Test via a statistical test (e.g., one-sided Wilcoxon rank sum test) whether, in dataset I, the inhibition of gene B is more lethal in samples in which gene A is overactive compared to the rest of the samples; gene overactivation is deduced from the genomic and optionally also from the expression profiles of the samples in dataset I;
   ii. Let SDL_functional_{pvalue,I}(A,B) be the obtained p-value;
   iii. If SDL_functional_{pvalue,I}(A,B)<0.05 add (A,B) to functional_{SDL};
   e. determining whether (A,B) is to be added to mRNA_{SL} and mRNA_{SDL}:
      for every dataset I ∈ expression_{datasets}
   i. compute the Spearman correlation between the expression of gene A and gene B in dataset I;
   ii. let expression_{pvalue,I}(A,B) be the correlation p-value, and expression_{correlation,I}(A,B) be the correlation coefficient;
   iii. if expression_{correlation,I}(A,B)≧Rₘᵢₙ, and expression_{pvalue,I}(A,B) following Bonferroni correction is below 0.05 add (A,B) to expression_{SL} and to expression_{SDL};
IV.
   a. creating an SL output network as the intersection of networks SoF_{SL}, functional_{SL}, and expression_{SL}, such that an edge exists in the combined graph only if it appears in the three graphs;
   b. creating an SDL output network as the intersection of graphs SoF_{SDL}, functional_{SDL}, and expression_{SDL}, such that an edge exists in the combined graph only if it appears in the three graphs;
V. for every inference procedure combine the p-values obtained by its datasets into a single p-value per gene-pair via Fisher's combined probability test:
   a. SL_SoFₚᵥₐₗᵤₑ(A,B)=Fisher's_Method({SL_SoF_{pvalue,I}(A,B)|I∈ SoF_{datasets}})
   b. SDL_SoFₚᵥₐₗᵤₑ(A,B)=Fisher's_Method({SDL_SoF_{pvalue,I}(A,B)|I∈ SoF_{datasets}})
   c. SL_functionalₚᵥₐₗᵤₑ(A,B)=Fisher's_Method({SL_functional_{pvalue,I}(A,B)|I∈functional_{datasets}})
   d. SDL_functionalₚᵥₐₗᵤₑ(A,B)=Fisher's_Method({SDL_functional_{pvalue,I}(A,B)|I∈functional_{datasets}})
   e. expressionₚᵥₐₗᵤₑ(A,B)=Fisher's_Method({expression_{pvalue,I}(A,B)|I∈expression_{datasets}})
VI. further integrated the three combined p-values into one p-value per gene-pair, again via Fisher's method, considering all inference procedures:
   SL_Allₚᵥₐₗᵤₑ(A,B)=Fisher's_Method(SL_SoFₚᵥₐₗᵤₑ(A,B)∪SL_functionalₚᵥₐₗᵤₑ(A,B)∪expressionₚᵥₐₗᵤₑ(A,B)})
   SDL_Allₚᵥₐₗᵤₑ(A,B)=Fisher's_Method(SDL_SoFₚᵥₐₗᵤₑ(A,B)∪SDL_functionalₚᵥₐₗᵤₑ(A,B)∪expressionₚᵥₐₗᵤₑ(A,B)})
VII. for each pair of genes (A,B) ∈ [GeneList×GeneList] return SL_SoFₚᵥₐₗᵤₑ(A,B), SL_functionalₚᵥₐₗᵤₑ(A,B), SDL_SoFₚᵥₐₗᵤₑ(A,B), SDL_functionalₚᵥₐₗᵤₑ(A,B), expressionₚᵥₐₗᵤₑ(A,B), and SL_Allₚᵥₐₗᵤₑ(A,B), SDL_Allₚᵥₐₗᵤₑ(A,B).

Each edge in the combined graph thus represents an interacting pair of genes, having a unified p-value.

According to some embodiments, once the graphs are available, they may be analyzed for retrieving information and assisting in taking decision relevant for the patient. Graphs may be analyzed in a supervised or non-supervised manner, wherein the graph is combined with a genetic profile of a patient's tumor.

The present invention provides according to one aspect, a method of applying SL and SDL networks for predicting the response of cancer cells to the inhibition of a gene product, based on the genomic profile of the cells. The latter can be a profile of SCNA, mutations, DNA or histone methylation, gene expression (mRNA) or protein abundance.

According to some embodiments, the method is utilized in an unsupervised mode wherein, 1) for each sample inactive and overactive genes are identified according to its genomic profile; and 2) the viability of a given sample is predicted following the inhibition of a given gene as proportional to the number of inactive SL-partners and overactive SDL-partners the pertaining gene has in the given sample.

According to other embodiments, the method is utilized in a supervised mode wherein, important features of the network and relevant genetic characteristics of the tumor are extracted and utilized to train and utilize machine learning predictors. The training of the predictors is done according to some embodiments by integrating experimental measurements of gene essentiality or drug efficacy. The machine learning predictors according to some embodiments are Support Vector Machine (SVM) classifiers or Neural Network predictors.

Some analyses may relate to identifying potential targets for therapy, while other analyses may relate to assessing prognosis for a patient.

In another example, the SL-network and/or the SDL network may be used to provide prognosis for the patient.

### Definitions

Synthetic lethality (SL) occurs when a perturbation of two nonessential genes is lethal.

Synthetic Dosage Lethality (SDL) denotes an interaction between two genes in which the over-activity of one gene renders the other gene essential.

SL-based treatment refer to treatment of a condition (such as, cancer) with known, repurposed or newly identified, agents capable of targeting at least one gene present in an SL or SDL network according to the present invention.

Somatic copy Number of Alterations (SCNA) refer to somatic changes to chromosome structure that result in gain or loss in copies of sections of DNA, and are prevalent in many types of cancer.

Messenger RNA (mRNA) is a large family of RNA molecules that convey genetic information from DNA to the ribosome, where they specify the amino acid sequence of the protein products of gene expression. mRNA genetic information is in the sequence of nucleotides, which are arranged into codons consisting of three bases each.

A small hairpin RNA or short hairpin RNA (shRNA) is a sequence of RNA that makes a tight hairpin turn that can be used to silence target gene expression via RNA interference (RNAi). Expression of shRNA in cells is typically accomplished by delivery of plasmids or through viral or bacterial vectors.

Small interfering RNA (siRNA), sometimes known as short interfering RNA or silencing RNA, is a class of double-stranded RNA molecules, 20-25 base pairs in length. siRNA plays many roles, but it is most notable in the RNA interference (RNAi) pathway, where it interferes with the expression of specific genes with complementary nucleotide sequences. siRNA functions by causing mRNA to be broken down after transcription, resulting in no translation.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, lung cancer (including small-cell lung cancer, non-small-cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic NHL; intermediate grade/follicular NHL; intermediate grade diffuse NHL; high grade immunoblastic NHL; high grade lymphoblastic NHL; high-grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

The term “anti-neoplastic composition” refers to a composition useful in treating cancer comprising at least one active therapeutic agent capable of inhibiting or preventing tumor growth or function or metastasis, and/or causing destruction of tumor cells. Therapeutic agents suitable in an anti-neoplastic composition for treating cancer include, but not limited to, chemotherapeutic agents, radioactive isotopes, toxins, cytokines such as interferons, and antagonistic agents targeting cytokines, cytokine receptors or antigens associated with tumor cells. For example, therapeutic agents useful in the present invention can be antibodies such as anti-HER2 antibody and anti-CD20 antibody, or small molecule tyrosine kinase inhibitors such as VEGF receptor inhibitors and EGF receptor inhibitors. Preferably the therapeutic agent is a chemotherapeutic agent.

A “chemotherapeutic agent” is a chemical compound useful in the treatment of cancer. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Agnew, Chem Intl. Ed. Engl. 33:183-186 (1994)); dynemicin, including dynemicin A; bisphosphonates, such as clodronate; an esperamicin; as well as neocarzinostatin chromophore and related chromoprotein enediyne antibiotic chromophores), aclacinomycins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine ; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, Aexemestane, formestanie, fadrozole, vorozole, letrozole, and Aanastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy DNA-based vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

The term “repurposing” is directed to repurposing known active ingredients which are used for treating a first condition in the therapy of a different condition, such as, cancer therapy.

### Experimental Procedures

### Description of DAISY

A method of identifying Synthetic Lethal (SL) and Synthetic Dosage Lethal (SDL)-interactions, and generating SL and SDL networks, using a direct data-driven computational system, is provided, wherein the computational system utilizes three types of profiles:
A gene-activity-profile, denoting the activity level of genes in a given cancer sample or cell line, according to the analysis of one or more of the following data types: Somatic Copy Number of Alterations (SCNA), germline Copy-Number Variations (CNV), DNA methylation, histone methylation, somatic or germline mutations; optionally, the gene-activity-profile can be further refined by accounting for the gene-expression-profile(s) (as described in (3)) of the cancer sample or cell line;
A gene-essentiality-profile, denoting the level of lethality measured following the inhibition of various genes in a given cancer sample or cell line; gene inhibition can be obtained via, for example, shRNA, siRNA, mutagenesis, or drug administration;
A gene-expression-profile, denoting either a transcriptomic profile or a protein abundance profile of a given cancer sample or cell line.
   The computational system identifies SL-pairs by applying the following statistical inference procedures for every pair of genes (gene A and gene B):
I. “genomic Survival of the Fittest” (SoF) examines if the co-inactivation of both genes (A and B) occurs significantly less than expected by analyzing gene-activity-profiles.
II. “inhibition-based functional examination” integrates the gene-activity-profiles of a set of cancer samples with the gene-essentiality-profiles of these samples, and examines if gene B is significantly more essential in samples in which gene A is inactive.
III. “pairwise gene co-expression”, examines if the expression of genes A and B is correlated, by analyzing gene-expression-profiles.
   Likewise, the computational system identifies SDL-pairs by applying the statistical inference procedure described in (III) as well as the following two procedures for every pair of genes (gene A and gene B):
IV. “genomic Survival of the Fittest” (SoF) examines if the over-activation of gene A along with the inactivation of gene B occurs significantly less than expected by analyzing gene-activity-profiles.
V. “inhibition-based functional examination” integrates the gene-activity-profiles of a set of cancer samples with the gene-essentiality-profiles of these samples, and examines if gene B is significantly more essential in samples in which gene A is overactive.

For each gene-pair five p-values are obtained according to each one of the statistical inference procedures described above. The p-values obtained in (I)-(III) denote the significance of the SL-interaction between the two genes, while the p-values obtained in (III)-(V) denote the significance of the SDL-interaction between the two genes. Gene-pairs with significantly low p-values (e.g., <0.01 following multiple hypotheses correction) are considered as predicted SL- or SDL-pairs.

The datasets utilized to detect SL- and SDL-interactions via DAISY are listed in Table 6. To construct the SL- and SDL-networks, the input GeneList for DAISY algorithm (see above) included 23,125 genes, and hence DAISY traversed over ˜535 million gene pairs. To do so efficiently DAISY was implemented based on the HTcondor architecture, which enables parallel computing (Thain et al., 2005).

A pseudo-code implementing DAISY is provided below.
1. creating and initializing the following graphs: SoF_{SL}, SoF_{SDL}, functional_{SL}, functional_{SDL}, expression_{SL}, and expression_{SDL}, wherein SoF_{SL} and SoF_{SDL} are the SL and SDL networks constructed from SoF_{data}, respectively; functional_{SL} and functional_{SDL} are the SL and SDL networks constructed from functional_{data}, respectively; expression_{SL} and expression_{SDL} are the SL and SDL networks constructed from the expression_{data}, respectively;
2. input description: In the following description a genetic profile denotes a profile that consists of one or more of the following data: Somatic Copy Number of Alterations (SCNA), germline Copy-Number Variations (CNV), DNA methylation, histone methylation, somatic or germline mutations; an expression profile denotes either a transcriptomic profile or a protein abundance profile. Given a set of genes whose SL and SDL-partners are to be found (termed GeneList), and three sets of data:
   a. SoF_{datasets} referring to datasets that will be utilized to generate the SoF_{SL} and SoF_{SL}, each dataset will include genomic profiles of a set of cancer samples, and optionally also the expression profiles of these samples;
   b. functional_{datasets} referring to dataset that will be utilized to generate the functional_{SL} and functional_{SDL}; each dataset will include the gene essentiality measurements taken from a cohort of cancer cell lines, along with the genomic profiles of these cell lines, and optionally also the expression profiles of these cell lines. Gene essentiality measurements can be obtained via shRNA, siRNA, or molecular inhibitors;
   c. expression_{datasets} referring to dataset that will be utilized to generate the expression_{SL} and expression_{SDL}; each dataset will include expression profiles of a set of clinical cancer samples or cancer cell lines;
3. for each pair of genes (A,B) ∈ [GeneList×GeneList]:
   a. determining whether (A,B) is to be added to SoF_{SL}:
      for every dataset I ∈ SoF_{datasets}
      i. test via a statistical test (e.g., one-sided Wilcoxon rank sum test) whether, in dataset I, gene B has higher SCNA levels in samples in which gene A is inactive compared to the rest of the samples; gene inactivation is deduced from the genomic and optionally also from the expression profiles of the samples in dataset I;
      ii. let SL_SoF_{pvalue,I}(A,B) be the obtained p-value;
      iii. if SL_SoF_{pvalue,I}(A,B) following Bonferroni correction is below 0.05 add (A,B) to SoF_{SL};
   b. determining whether (A,B) is to be added to SoF_{SDL}:
      for every dataset I ∈ SoF_{datasets}
      i. test via a statistical test (e.g., one-sided Wilcoxon rank sum test) whether, in dataset I, gene B has higher SCNA levels in samples in which gene A is overactive compared to the rest of the samples; gene overactivation is deduced from the genomic and optionally also from the expression profiles of the samples in dataset I;
      ii. let SDL_SoF_{pvalue,I}(A,B) be the obtained p-value;
      iii. if SDL_SoF_{pvalue,I}(A,B) following Bonferroni correction is below 0.05 add (A,B) to SoF_{SDL};
   c. determining whether (A,B) is to be added to functional_{SL}:
      for every dataset I ∈ functional_{datasets}
      i. test via a statistical test (e.g., one-sided Wilcoxon rank sum test) whether, in dataset I, the inhibition of gene B is more lethal in samples in which gene A is inactive compared to the rest of the samples. gene inactivation is deduced from the genomic and optionally also from the expression profiles of the samples in dataset I;
      ii. let SL_functional_{pvalue,I}(A,B) be the obtained p-value;
      iii. if SL_functional_{pvalue,I}(A,B)<0.05 add (A,B) to functional_{SL};
   d. determining whether (A,B) is to be added to functional_{SDL}:
      for every dataset I ∈ functional_{datasets}
      i. Test via a statistical test (e.g., one-sided Wilcoxon rank sum test) whether, in dataset I, the inhibition of gene B is more lethal in samples in which gene A is overactive compared to the rest of the samples; gene overactivation is deduced from the genomic and optionally also from the expression profiles of the samples in dataset I;
      ii. Let SDL_functional_{pvalue,I}(A,B) be the obtained p-value;
      iii. If SDL_functional_{pvalue,I}(A,B)<0.05 add (A,B) to functional_{SDL};
   e. determining whether (A,B) is to be added to mRNA_{SL} and mRNA_{SDL}:
   for every dataset I ∈ expression_{datasets}
      i. compute the Spearman correlation between the expression of gene A and gene B in dataset I;
      ii. let expression_{pvalue,I}(A,B) be the correlation p-value, and expression_{correlation,I}(A,B) be the correlation coefficient;
      iii. if expression_{correlation,I}(A,B)≧Rₘᵢₙ, and expression_{pvalue,I}(A,B) following Bonferroni correction is below 0.05 add (A,B) to expression_{SL} and to expression_{SDL};
4.
   a. creating an SL output network as the intersection of networks SoF_{SL}, functional_{SL}, and expression_{SL}, such that an edge exists in the combined graph only if it appears in the three graphs;
   b. creating an SDL output network as the intersection of graphs SoF_{SDL}, functional_{SDL}, and expression_{SDL}, such that an edge exists in the combined graph only if it appears in the three graphs;
5. for every inference procedure combine the p-values obtained by its datasets into a single p-value per gene-pair via Fishers combined probability test (Mosteller and Fisher):
   a. SL_SoFₚᵥₐₗᵤₑ(A,B)=Fisher's_Method({SL_SoF_{pvalue,I}(A,B)|I∈ SoF_{datasets}})
   b. SDL_SoFₚᵥₐₗᵤₑ(A,B)=Fisher's_Method({SDL_SoF_{pvalue,I}(A,B)|I∈ SoF_{datasets}})
   c. SL_functionalₚᵥₐₗᵤₑ(A,B)=Fisher's_Method({SL_functional_{pvalue,I}(A,B)|I∈functional_{datasets}})
   d. SDL_functionalₚᵥₐₗᵤₑ(A,B)=Fisher's_Method({SDL_functional_{pvalue,I}(A,B)|I∈functional_{datasets}})
   e. expressionₚᵥₐₗᵤₑ(A,B)=Fisher's_Method({expression_{pvalue,I}(A,B)|I∈expression_{datasets}})
6. further integrated the three combined p-values into one p-value per gene-pair, again via Fisher's method, considering all inference procedures:
   SL_Allₚᵥₐₗᵤₑ(A,B)=Fisher's_Method(SL_SoFₚᵥₐₗᵤₑ(A,B)∪SL_functionalₚᵥₐₗᵤₑ(A,B)∪expressionₚᵥₐₗᵤₑ(A,B)})
   SDL_Allₚᵥₐₗᵤₑ(A,B)=Fisher's_Method(SDL_SoFₚᵥₐₗᵤₑ(A,B)∪SDL_functionalₚᵥₐₗᵤₑ(A,B)∪expressionₚᵥₐₗᵤₑ(A,B)})
7. for each pair of genes (A,B) ∈ [GeneList×GeneList] return SL_SoFₚᵥₐₗᵤₑ(A,B), I SL_functionalₚᵥₐₗᵤₑ(A,B), SDL_SoFₚᵥₐₗᵤₑ(A,B), I SDL_functionalₚᵥₐₗᵤₑ(A,B), expressionₚᵥₐₗᵤₑ(A,B), and SL_Allₚᵥₐₗᵤₑ(A,B), SDL_Allₚᵥₐₗᵤₑ(A,B).

### Evaluating DAISY Based on Experimentally Detected SL-Interactions

The fit between the SL-pairs identified by DAISY, and those detected in six independent SL-screens that were conducted in cancer cell lines was tested: (1) An shRNA screen of 88 kinases conducted in renal carcinoma cells to identify the SL-partners of VHL (Bommi-Reddy et al., 2008); (2) a screen of a small molecule library encompassing 1,200 drugs and drug-like molecules that identified agents selectively lethal to endometrial adenocarcinoma cells lacking functional MSH2 (Martin et al., 2009); (3-4) two high-throughput RNA interference (RNAi) screens that identified determinants of sensitivity to a PARP1-inhibitor in breast cancer among (3) DNA repair genes (Lord et al., 2008), and (4) kinases (Turner et al., 2008); (5) a genome-wide shRNA screens (Luo et al., 2009) and (6) a large-scale siRNA screen (Steckel et al., 2012) that identified genes selectively essential to KRAS-transformed colon cancer cells, but not to derivatives lacking this oncogene.

DAISY was applied to identify the SL-partners of VHL, MSH2 and PARP1, and the SDL-partners of KRAS. DAISY examined gene pairs that were experimentally examined in one of the screens described above. In the case of KRAS, for which two large-scale screens were conducted, DAISY examined only genes that were tested in both screens as potential KRAS SDL-partners. A gene was considered to be an experimentally identified KRAS-SDL only if it was detected as a KRAS-SDL in both screens. For MSH2, we mapped between the drugs that were utilized in the screen to their targets according to DrugBank (Knox et al., 2011), and disregarded drugs with more than one target, to avoid ambiguity.

To rigorously evaluate DAISY's performances in identifying the SL- and SDL-partners of these key cancer-associated genes, the p-values DAISY generated were used in an unsupervised manner, between SDL or SL (SDL/SL) and non-SDL/SL gene pairs. DAISY computed for every dataset and every pair of genes a p-value that denotes the significance of the association between the genes according to the pertaining dataset (prior to the correction for multiple hypotheses testing). For every data-type the p-values obtained by its datasets were combined into a single p-value per gene-pair via Fisher's combined probability test, also known as Fisher's Method (Mosteller and Fisher, 1948).

The p-values were corrected for multiple hypotheses testing via Bonferroni correction, and used to classify the gene-pairs along an increasing cutoff that defined which p-values are small enough to conclude that a gene-pair is interacting. Based on the latter ROC curves were generated, which plot the true positive rate vs. the false positive rate of the prediction across various decision threshold settings. The prediction was evaluated based on the AUC of the ROC. An empirical p-value were computed for the obtained AUC by randomly shuffling the labels 10,000 times, and re-computing the AUC with the random labels. The number of times a random AUC was greater or equal to the original AUC was then counted. This number divided by 10,000 is the empirical p-value of the ROC.

### Examining the SL-Network Based on Gene Essentiality Data

The utility of an SL-network can be examined by employing it to predict gene essentiality in a cell-line-specific manner, and testing whether these predictions are supported by experimental results obtained in shRNA screens. The procedure requires one to define two parameters:
Deletion_{cutoff}—the SCNA level under which a gene is considered deleted.
SLessentiality_{cuttoff}—the minimal number of inactive SL-partners that renders a gene essential.
   Given these parameters the procedure is performed as follows, for every cell line: (1) Underexpressed genes that have an SCNA level below Deletion_{cutoff} are defined as inactive; (2) the number of inactive SL-partners of each gene denotes its predicted essentiality; (3) genes with at least SLessentiality_{cuttoff} inactive SL-partner are predicted as essential.

To validate the SL-network in this manner it was first reconstructed without the shRNA datasets, to avoid any potential circularity. It was employed to predict the essentiality of 1,288 SL-network-genes in 46 cancer cell lines. For these cell lines both gene expression and SCNA data were used to generate the predictions, and gene essentiality data for validation (Barretina et al., 2012; Marcotte et al., 2012). Deletion_{cutoff} was defined as −0.1, based on the literature (Beroukhim et al., 2010), and the SLessentiality_{cuttoff} as 1—a gene is said to be essential in a cell line if at least one of its SL pairs is deleted. Underexpression was defined as previously explained (expression below the 10^{th} percentile of this gene across samples). The range of Deletion_{cutoff} and SLessentiality_{cuttoff} parameters was examined, demonstrating the robustness of the SL-network performances.

The gene essentiality predictions were examined based on the experimental zGARP scores (Marcotte et al., 2012). The lower the zGARP score is, the more essential the gene is. The examination process was performed as follows.
1. For each cell line four p-values were obtained:
   a. Two one-sided Wilcoxon rank sum p-values, denoting whether the zGARP scores of the predicted essential genes are significantly lower than those of genes predicted as nonessential, when considering all genes or only SL-network genes as the background model.
   b. Two hypergeometric p-values, denoting if the predicted essential genes are significantly enriched with experimentally identified essential genes, when considering all genes or only SL-network genes as the background model. A gene was defined a as experimentally essential if its zGARP score in a given cell line was below −1.289 (the 10^{th} percentile of the zGARP scores) (Marcotte et al., 2012).
2. According to each one of these four p-values, the number of cell lines for which the predictions significantly match the experimental findings (p-value<0.05), were computed.

To examine the significance of the results obtained by the SL-network gene-essentiality was predicted based on 10,000 random networks of the same topology as SL-network Based on the performances of the random networks four empirical p-values were obtained, each denoting if the performance of the SL-network is significant according to one of the four original p-values described in (1) above.

### Examining the SDL-Network Based on Drug Efficacy Measurements

The validity of the SDL-network was evaluated by employing it to predict the sensitivity of different cancer cell lines to various drugs, and to compare the predictions to drug efficacy measurements. The procedure is based on two parameters:
Overexpression_{cutoff}—a threshold for identifying overexpressed genes. For every gene the Overexpression_{cutoff} percentile of its expression level across the different samples in the dataset, was computed and defined a gene as overexpressed if its expression is above this percentile.
SDLessentiality_{cuttoff}—the number of overexpressed SDL-partners that renders a gene essential.

Given these two parameters, for every cell line: its overexpressed genes were identified, predicted genes with at least SDLessentiality_{cuttoff} overexpressed SDL-partner as essential, and predicted the cell line as sensitive to drugs whose targets were predicted as essential in it. For each drug it was tested whether its efficacy is higher in the cell lines that were predicted as sensitive compared to its efficacy in cell lines that were predicted as resistant (one-sided Wilcoxon rank sum test). The fraction of drugs for which the network significantly differentiates (p-value<0.05) between sensitive and resistant cell line was then computed. The process of drug efficacy predictions was repeated based on 10,000 random networks of the same topology as the SDL-network, and empirical p-values were obtained, denoting the significance of SDL-network performances in this task.

To evaluate the SDL-network in this manner, the data from the CGP (Garnett et al., 2012) and from the CTRP (Basu et al., 2013) was used. The CGP data contains the IC50 values of 131 drugs across 639 cancer cell lines. (The IC50 of a drug denotes the drug concentration required to eradicate 50% of the cancer cells.) The CTRP data includes the sensitivities of 242 cancer cell lines to 354 small molecules. The sensitivity measure in this case is termed area-under-the-dose-curve. Gene expression profiles of 593 out of the 639 cell lines used in the CGP data, and the expression profiles of 241 cell lines used in the CTRP from the Cancer Cell Line Encyclopedia (CCLE) (Barretina et al., 2012) were extracted. As the method exploits the SDL-network to deduce the efficacy of each drug in a given context, it was possible to perform the prediction only for drugs that had at least one of their targets in the SDL-network—37 and 49 drugs in the CGP and CTRP data, respectively. The drugs were mapped to their targets based on the mapping reported in the CGP and in the CTRP, and based on DrugBank (Basu et al., 2013; Garnett et al., 2012; Knox et al., 2011).

The parameters were set to an Overexpression_{cutoff} of 80, and an SDLessentiality_{cuttoff} of 2. Under these definitions, it was possible to predict the response of cells only to drugs that had targets with at least two SDL-partners—23 and 32 drugs in the CGP and CTRP data, respectively. The sensitivity of the predictions to the Overexpression_{cutoff} and SDLessentiality_{cuttoff} parameters was examined, demonstrating the robustness of the network. Lastly, to evaluate single SDL-interactions, this analysis was repeated for each SDL pair alone, instead of using the entire SDL-network.

### Supervised Learning: Data Description

Two types of neural network models were constructed. The first model predicts a gene-cell line pair relation—whether a gene is essential in a specific cancer cell line or not. The second model predicts a drug-cell line pair relation—the efficacy of a drug in a given cell line. Both models used a set of 53 features, based on the SL/SDL-networks.

The first model is given a set of features, which define a gene-cell line pair, and predicts if the gene is essential in the cancer cell line or not. To generate the features the SL-network that was reconstructed without the shRNA datasets was utilized, to avoid any potential circularity. This was employed to predict the essentiality of 1,288 SL-network-genes in 46 cancer cell lines (the network can be used to predict only the essentiality of the genes it contains). For these 46 cell lines the data required to generate the features—gene expression and SCNA data—was obtained from the CCLE (Barretina et al., 2012). Gene essentiality data was taken from (Marcotte et al., 2012). Each gene-cell line pair was represented based on the 53 features (see section below). If the zGARP score of the gene in the cell line was below −1.289 (below the 10^{th} percentile of the zGARP scores), it was denoted as essential in this cell line, and the pair was labeled as 1, otherwise it was labeled −1 (that is, non-essential). The prediction was performed for 47,978 gene-cell line pairs, 6,066 (12.6%) of which were labeled as 1, and the rest as −1 (11,270 pairs were omitted due to the lack of data).

The second type of models obtained were given a set of features that define a drug-cell line pair, and predicted the efficacy of the drug when administered to the cell line. Such models were obtained for each of the pharmacologic datasets separately: (1) Models that predicts log IC50 values and are trained and tested based on the CGP data (Garnett et al., 2012), and (2) models that predicts the area-under-the-dose-curve and are trained and tested based on the CTRP data (Basu et al., 2013). The features were generated based on the SDL-network and the genomic profiles of the cell lines (see next section). To generate the features from the CCLE the gene expression and SCNA profiles of 414 and 241 of the cell lines used in the CGP and CTRP data, respectively were extracted. As the method exploits the SDL-network to deduce the efficacy of each drug in a given context, it was possible to perform the prediction only for drugs that had at least one of their targets in the SDL-network—37 and 49 drugs in the CGP and CTRP data, respectively. For the CGP data the resulting matrix of 414 cell lines by 37 drugs contains 8,814 IC50 values, with 6,504 missing values; overall there were 8,770 drug-cell line pairs, as 44 pairs were removed due to the lack of genomic data (i.e., missing mRNA or SCNA data). For the CTRP data the resulting matrix of 244 cell lines by 37 drugs contains 8,170 efficacy values, with 3,639 missing values; overall 7,890 drug-cell line pairs were identified, as 294 pairs were removed due to the lack of genomic data.

### Supervised Learning: Features

53 features that describe the state of a given gene in a given cell line were extracted based on the SL-network combined with SCNA and mRNA data:
1. The number of inactive SL-partners or overactive SDL-partners the gene has in the cell line. (A gene is defined as inactive if it is underexpressed and its SCNA level is below −0.3, and as overactive if it is overexpressed and its SCNA level is above 0.3).
2-13. The sum, average, minimal, and maximal level of the gene's SL/SDL-partners in the cell line, according to SCNA, mRNA, and normalized mRNA measurements. (The mRNA measurements were normalized via z-score, such that the mean and standard deviation of the expression of each gene across the samples are 0 and 1, respectively).
14-25. The sum, average, minimal, and maximal level of the gene's SL/SDL-partners across all cell lines, according to SCNA, mRNA, and normalized mRNA measurements.
26-27. The mRNA and SCNA level of the gene in the cell line, times the number of inactive SL-partners or overactive SDL-partners it has.
28-37. Principle Component Analysis (PCA) was performed with the adjacency matrix of the network. As the network is directional and not symmetric PCA was also performed with the transpose of the networks adjacency matrix The five first principle components of the gene based on each one of the matrixes were then used.
38-39. The in- and out-degree of the gene in the network.
40-45. The average, minimal and maximal SCNA and mRNA levels of the gene across the different cell lines.
46-47. The mRNA and SCNA level of the gene in the cell line.
48-53. The average, minimal and maximal mRNA and SCNA levels measured in the cell line.

To predict the drug efficacy in various cancer cell lines these gene-cell features were transformed to drug-cell features. To this end the drug and its target genes were mapped, and the drug-cell features were computed as an average of the (target) gene-cell feature. The mapping between drugs and their targets was taken from the CGP, the CTRP, and DrugBank (Basu et al., 2013; Garnett et al., 2012; Knox et al., 2011).

### Supervised Learning: Neural Networks

Neural network predictors were built by employing the MATLAB implementation of a feed-forward multi-layer perceptron (the function ‘fitnet’) with the default parameters. Three different layers were defined: input, hidden and output layer. The number of features (53, see above) determined the number of input units. The number of hidden units was 20. The sigmoid function was used as the perceptron activation function of the neural network model. A 5-fold cross-validation was performed for building the models: The original dataset was separated into five equally sized sets, obtained by randomly distributing all gene-cell or drug-cell pairs into five sets. In the discretized form (gene-cell) each set had the same ratio between positive and negative samples as in the full dataset. In each iteration one of the sets was exclusively used for testing, while others were destined for training the model.

### Utilizing the SL-Network to Predict Prognosis in Breast Cancer

The gene-expression profiles of 2,000 breast cancer clinical samples were utilized to examine the prognostic-value embedded in the SL-network (Curtis et al., 2012). Samples whose survival status was ambiguous or unknown were disregarded, resulting in 1,586 samples. Based on the gene expression of each one of the SL-pair two groups of patients were defined:
1. The low group: The group in which both of the SL-paired genes are lowly expressed (that is, below the median of the gene expression levels).
2. The high group: The group in which at least one of the SL-paired genes is expressed (that is, above the median of the gene expression levels).

For each SL-pair the 15-year survival Kaplan-Meier plots of its two groups of patients were generated, and a log rank p-value was obtained denoting the significance of the separation between the two groups in terms of their prognosis (Bland and Altman, 2004). In addition, a signed KM-score was defined, whose magnitude (absolute value) is −log(p-value), and hence the more significant the log rank p-value is the higher the magnitude of the signed KM-score will be. The sign of the signed KM-score is positive if the low group had a better prognosis, and negative otherwise. The rationale behind the signed KM-score is that it is assumed that the SL-pairs not only significantly separate between groups of patients in respect to their prognosis (as reflected by the log rank p-value), but do so in a directional manner: the low group would have a better prognosis as compared to the high group. This directionality is reflected in a positive signed KM-score.

To evaluate the performance of the SL-pairs it was compared to the performance of single SL-network-genes and to that of two groups of 10,000 randomly selected gene-pairs: (a) Those that consist only of SL-network-genes, and (b) those that consist of all genes. When working with single genes the low group consisted of samples that underexpressed the gene, and the high group consisted of samples that expressed the gene. The results (log rank p-values and signed KM-scores) obtained with the original SL-network pairs were then compared to the results obtained with each of the three groups (single SL-network genes and the two types of randomly selected pairs) via a one-sided Wilcoxon rank sum test.

For each SL-pair of genes Cox-regression was performed to evaluate whether its prognostic value is significant even when accounting for the following clinical characteristics of the breast cancer patients: Age at diagnosis, grade, tumor size, lymph nodes, estrogen receptor expression, HER2 expression, and progesterone receptor expression. Correction for multiple hypothesis testing was done based on the Benjamini-Hochberg algorithm (Benjamini and Hochberg, 1995).

Lastly, the patients were classified according to the overall SL-network behavior. That is, instead considering only the expression of a specific SL-pairs, the expression of the entire set of SL-pairs were considered. To do so it was computed for each sample how many of the SL-pairs in the network it co-underexpressed, and defined a global SL-score being the fraction of SL-pairs that were classified to the low group. As a random model two types of random networks were generated, of the same topology as the SL-network that consisted of: (1) essential genes in breast cancer—1,971 genes that obtained the lowest average zGARP score measured in 29 breast cancer cell lines (Marcotte et al., 2012), (2) deletion driver genes—1,971 genes that obtained the lowest q-value in an analysis which identified deletion drivers (Beroukhim et al., 2010). Both random networks include 1,971 genes, as the original SL-network includes 1,971 genes. In this analysis random networks that consist of the SL-network genes were not used as a random model as the SL-scores of such networks are highly correlated with the SL-scores of the original network (mean Spearman correlation coefficient of 0.927). 10,000 random networks of each type were generated as described above. Based on each one of these networks the global SL-scores for each sample was computed and the samples were divided into four groups according to these scores (the first, second, third, and fourth groups include samples with a global SL-score that is between the 0-25th, 25th-50th, 50th-75th, and 75th-100th percentiles of the scores, respectively). For each random network a log rank p-value was then computed, denoting if the 15-year survival of the four groups is significantly different. It was also examined if the order of the four groups is as expected, that is, if the groups with higher global SL-scores had better 15-year survival. The number of random networks that obtained a log rank p-value which is at least as low as that obtained by the original network, was then counted, and also had the right order of groups in terms of survival. This number divided by 20,000 is the empirical p-value denoting the significance of the performances of the original SL-network in correctly dividing the samples based on their global SL-scores.

### Results

### The DAta-mIning SYnthetic-lethality-identification Pipeline (DAISY)

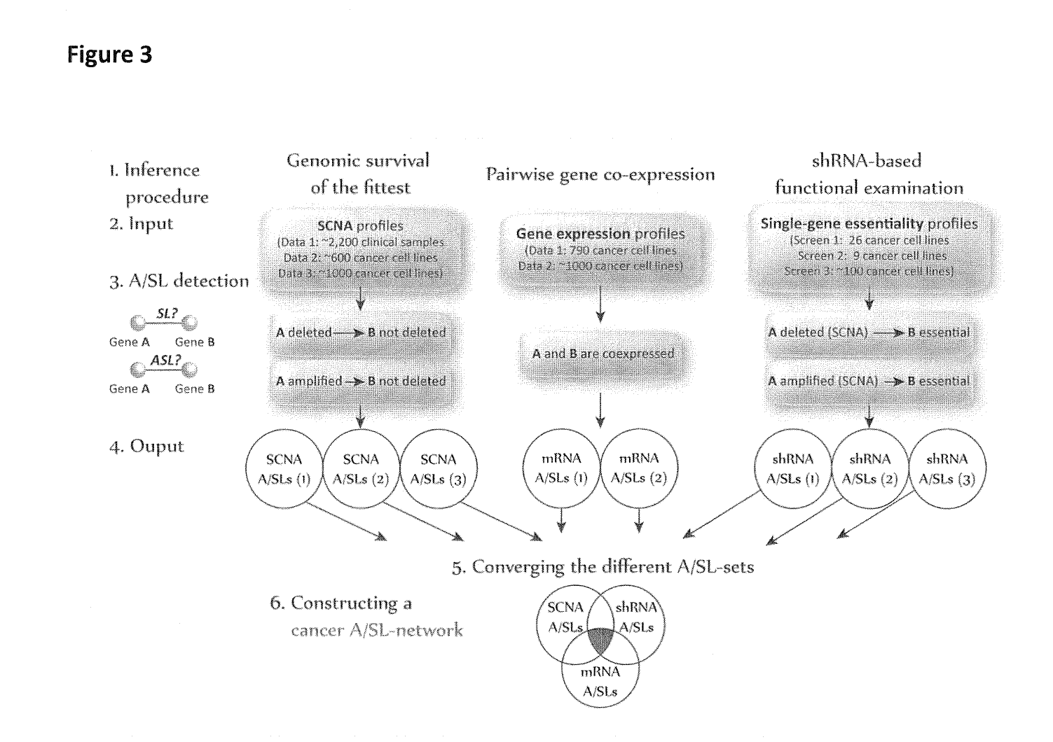

A new approach for inferring SL-interactions from cancer genomic data, collected from both cell-lines and clinical samples, termed DAISY, was developed. DAISY analyzes three data types: (1) Somatic Copy Number Alterations (SCNA), (2) phenotypic lethality data obtained in shRNA gene knockdown screens, and (3) gene expression (FIG. 3). The new approach applies three statistical inference procedures, each tailored to a specific dataset:
(1) The first, “genomic survival of the fittest”, is based on the observation that cancer cells that have lost two SL-paired genes will be strongly selected against. Accordingly, SL-interactions can be identified by analyzing SCNA data somatic mutation data and detecting events of gene-co-deletions that occur significantly less than expected. This is because cells harboring such SL co-deletions are eliminated from the population observed. In fact, very similar conceptual approaches are already extensively used to analyzed the outcomes of shRNA screens in cell lines, in which essential genes and SL-gene-pairs are detected by identifying the shRNA probes that have been rapidly eliminated from the cell population (Cheung et al., 2011; Luo et al., 2008; Marcotte et al., 2012).
(2) The second inference strategy, “shRNA based functional examination”, is closely related to the first. It is based on the notion that the essentiality of a synthetically lethal gene will manifest itself when it is knocked down in cancer cells where its SL-partner(s) are inactive (that is, with a markedly low copy-number and expression). Accordingly, the SL-pairs of a given gene can be identified by searching for genes whose underexpression and low copy-number induce its essentiality.
(3) The third procedure, “pairwise gene co-expression”, is based on the notion that SL-pairs tend to participate in closely related biological processes and hence are likely to be co-expressed (Costanzo et al., 2010; Kelley and Ideker, 2005). It is further shown herein that this trend indeed holds in known SLs that have been experimentally detected in cancer (FIG. 4).

Given SCNA, shRNA, and gene co-expression data of thousands of cancer samples, DAISY identifies SL-pairs by combining these three inference strategies. It traverses over all the possible gene-pairs (˜534 million), and examines for each pair if it fulfills the three statistical inference criteria expected from an SL-pair according to each one of the datasets, as described above. Gene-pairs that fulfill all the three criteria in a statistically significant manner are predicted by DAISY as SL-pairs. DAISY was applied to analyze eight different genome-wide cancer datasets (Barretina et al., 2012; Beroukhim et al., 2010; Cheung et al., 2011; Garnett et al., 2012; Luo et al., 2008; Marcotte et al., 2012) (FIG. 3, Barretina et al. and Beroukhim et al. each contains two datasets).

TABLE 6
Data description

| | | | No. | |
|---|---|---|---|---|
| | Data | Additional | clinical | |
| Type | type | data | samples | Reference |
| Clinical | SCNA | — | 2,201 | (Beroukhim |
| samples | | | | et al., 2010) |
| Cancer | SCNA | — | 591 | (Beroukhim |
| cell lines | | | | et al., 2010) |
| | SCNA | mRNA | 995 | The Cancer Cell |
| | | | | Line Encyclopedia |
| | | | | (CCLE) (Barretina |
| | | | | et al., 2012) |
| | mRNA | — | 790 | (Garnett |
| | | | | et al., 2012) |
| | mRNA | — | 997 | CCLE (Barretina |
| | | | | et al., 2012) |
| | shRNA | SCNA and | 91 | Achilles |
| | | mRNA profiles | | (Cheung |
| | | (Barretina | | et al., 2011) |
| | | et al., 2012) | | |
| | shRNA | SCNA and | 26 | (Marcotte |
| | | mRNA profiles | | et al., 2012) |
| | | (Barretina | | |
| | | et al., 2012) | | |
| | shRNA | SCNA profiles | 9 | (Luo |
| | | (Beroukhim | | et al., 2008) |
| | | et al., 2010) | | |

The concept of synthetic lethality was additionally expanded to encompass Synthetic Dosage Lethal (SDL) gene-pairs. While two genes form a regular SL pair if the inactivation of one gene renders the other essential, two genes form an SDL-pair if the amplification or over-activity of one of them renders the other gene essential. Importantly, SDL-interactions can permit the targeting of cancer cells with over-active oncogenes that are difficult to target directly (such as KRAS), by targeting the SDL-partners of such oncogenes. Their detection via DAISY is analogous to the way regular SLs are detected, using the same three inference procedures outlined above. More specifically, DAISY detects two genes, A and B, as an SDL-pair if their expression is correlated, and if the amplification or overexpression of gene A induces the essentiality of gene B. Induced essentiality is detected in two ways: first, according to shRNA screens, by examining if gene B become essential when gene A is overactive. Second, according to SCNA data, by examining if gene B has a higher SCNA level when gene A is overactive, potentially compensating for the over-activity of gene A.

### Evaluating DAISY Based on Experimentally Detected SL-Interactions in Cancer

As a first step in testing, DAISY SL predictions were generated for four central cancer genes for which there are already published experimentally-determined cancer SL-collections (there are yet only just a few such reports). DAISY was applied to identify the SL-partners of PARP1, the tumor suppressors VHL, and MSH2, and the SDL-partners of the oncogene KRAS. Using DAISY a predictor was built that classified every potential gene pair as either being an SL/SDL-pair or not, and compared these predictions to the experimental results that have been reported in six pertaining large-scale screens (Bommi-Reddy et al., 2008; Lord et al., 2008; Luo et al., 2009; Martin et al., 2009; Steckel et al., 2012; Turner et al., 2008). The performances of the DAISY-predictor were quantified based on the Area Under the Curve (AUC) of its Receiver Operating Characteristic (ROC) curve. The ROC-curve plots the fraction of true positives out of the total actual positives (TPR, true positive rate) vs. the fraction of false positives out of the total actual negatives (FPR, false positive rate) across many decision threshold settings. The resulting AUC is the standard measure of the overall performance of a classifier, where an AUC of 0.5 denotes the performance of a random predictor and an AUC of 1 denotes the performance of an ideal predictor.

Overall, the DAISY-predictor obtained an AUC of 0.799, which shows good concordance between the predicted and observed SL/SDLs (empirical p-value<1e-4, FIG. 4A). To assess which of the data types and inference strategies enables DAISY to successfully predict synthetic lethality, the predictions were also repeated when using only one data type at a time (Experimental Procedure). As shown in FIG. 4A, an AUC of 0.705 can be obtained by predicting SL-interactions only based on the SCNA genomic data. These results can be further improved by adding the gene expression data, reaching to an AUC of 0.790. As the shRNA data is not predictive on its own (AUC of 0.477), DAISY was modified to consider the shRNA criterion as a soft constraint (Experimental Procedures). Importantly, DAISY captures well-established and clinically important SL-interactions including the prominent SL-interaction between PARP1 and BRCA1/2 (Lord et al., 2008) and the synthetic lethality between MSH2 and DHFR (Martin et al., 2009). Reassuringly, a close examination of the SCNA and gene expression of these known SL-pairs measured in these datasets shows that the levels of one gene are significantly higher when its partner is deleted and that their expression is significantly correlated, as assumed by DAISY (FIG. 4B, C).

### Experimentally Examining DAISY Predicted SL-Partners of the Tumor Suppressor VHL

Some of the SL predictions were tested experimentally. The tumor suppressor VHL, which is frequently mutated in cancer, especially in clear cell renal carcinomas (Bommi-Reddy et al., 2008) was chosen as a model. DAISY was applied to predict the SL-partners of VHL and identify among these genes those which are essential in renal carcinoma cells (RCC4) exclusively due to the loss of VHL, resulting in a set of 44 genes.

An siRNA screen was performed to examine if the predicted genes are preferentially essential in VHL−/− renal carcinoma cells compared with isogenic cells in which pVHL function was restored (VHL+ cells). For each of the 44 target genes the inhibitory effect of its knockdown was measured in the two cell lines (each in six replicates), and its selectivity was quantified by a differential inhibition score (i.e., the percentage of growth inhibition observed in the VHL-deficient cells minus the percentage of growth inhibition observed in the VHL-restored cells).

Nine genes (20.45%) show a strong selective effect (differential inhibition score>10). One of the predicted genes (MYT1) has been previously identified as an SL-partner of VHL in a screen that searched for the SL-partners of VHL among 88 kinases (Bommi-Reddy et al., 2008). Hence, by treating this gene as a positive control anchor, it was possible to compare between this screen and the screen of Bommi-Reddy et al. In the present screen, the inhibition of 45.4% of the genes was at least as selective as the inhibition of MYT1. For comparison, only 11.9% of the genes examined in the Bommi-Reddy et al. screen have this property. Hence, according to this joint positive control, the present screen was able to find 3.83 times more SL genes than the previous screen (Bernoulli p-value of 4.758e-09).

DAISY predictions were further tested by measuring the response of the renal cells to 9 drugs whose targets were predicted by DAISY to be selectively essential in the VHL-deficient renal cells. A range of concentrations for each drug were tested to identify a suitable working concentration in which there was an effect on cells growth, but not complete death (which is more likely to be due to non-specific toxicity). The percentage of growth inhibition obtained at this mid-effective concentration of each drug on both cell lines (each in triplicates) was then measured. For all 6 drugs for which effects on cell growth could be identified, the VHL-deficient cells were more sensitive (higher percentage of inhibition at mid-effective concentration, FIG. 5). This specificity was however not observed with the positive control drug Staurosporine, indicating that the selective effect is not due to a general susceptibility of the VHL-deficient cells.

### Applying DAISY to Construct a Genome-Wide Network of SL-Interactions in Cancer

DAISY was applied to identify all gene pairs that are likely to be synthetically lethal in cancer, constructing the resulting data-driven cancer SL-network. As each of the eight datasets examined was analyzed separately the mutual overlap between the resulting SL-sets could be tested, and find to be significantly higher than expected by random. The resulting SL-network consists of 1,971 genes and 2,600 SL-interactions. It displays scale-free like characteristics, and is enriched with known cancer-associated genes, including drug targets, driver genes, oncogenes and tumor suppressors. The network is also significantly enriched with 152 Gene Ontology (GO) annotations (p-value<0.05 following multiple hypotheses correction), the top ones being cell cycle and division, mitosis, nuclear division, M phase, organelle fission, DNA metabolic processes, and DNA replication. The network clusters into six main clusters, each highly enriched with biological functions relevant to cancer.

### SL-Based Prediction of Gene Essentiality in Cancer Cell Lines

The utility of the networks in making functional predictions of interest in cancer was examined. Two prediction assignments were checked: the prediction of gene essentiality and the prediction of drug efficacy. In both tasks the SL/SDL-networks are utilized to generate cancer-specific predictions given a genomic characterization of a specific cancer in hand.

The SL-network was utilized to predict gene essentiality per cell line. As the predictions were aimed to be examined based on the results obtained in an shRNA gene knockdown screen, an SL-network was constructed for this test based only on mRNA and SCNA data, to avoid any potential circularity. Based on the latter, the cell-specific essentiality prediction proceeds in an unsupervised manner in two steps as follows: (1) First, for each cell line a list of inactive genes was determine. These are underexpressed genes whose SCNA level is below a certain Deletion_{cutoff} parameter (Experimental Procedure). (2) Second, to predict the viability of the cell line after the knockdown of a specific target gene X, the number of inactive SL-partners of X in the given cell line was compute. If their number is above a certain threshold (SLessentiality_{cutoff}), the knockdown of gene X in that cell line was predict to be lethal, and if not, it was predict to be viable. The results presented are based on setting the Deletion_{cutoff} as −0.1 following (Beroukhim et al., 2010), and the SLessentiality_{cuttoff} as 1, that is, assuming that a single SL-pair is lethal if indeed materialized. However, the results over a range of Deletion_{cutoff} and SLessentiality_{cuttoff} parameters demonstrate the robustness of the SL-network performance of the present invention over a broad range of cutoff values.

Using the approach described above gene essentiality was predicted in overall 129 different cancer cell lines, and examined the predictions based on the results obtained in two large-scale gene essentiality screens (Cheung et al., 2011; Marcotte et al., 2012). It was found that per cell line the predicted essential genes are enriched with experimentally determined essential genes and have significantly lower experimental essentiality scores in the given cell line (essential genes have lower scores, empirical p-value<2.52e-4, FIG. 5A, Experimental Procedures). Furthermore, the higher the number of predicted inactive SL-partners a gene has the more essential it is according to the experimental data (Spearman correlation coefficients of 0.996, and 0.942, p-values of 6.56e-72 and 1.86e-23, for the Marcotte and Achilles (Cheung et al. 2011) screens, respectively, FIGS. 6A-B). Of note, the SL-network succeeds more in predicting gene essentiality in cell lines with a higher number of gene deletions. Indeed, in such genetically unstable cell lines it is more likely that gene essentiality arises due to synthetic lethality. Finally, the SL-based gene essentiality prediction procedure described above was repeated, but this time replacing the SLs generated by DAISY with SLs that are human orthologs of yeast SLs (Conde-Pueyo et al., 2009). This however leads to markedly inferior performance, testifying to the inherent value embedded in the DAISY-inferred SLs.

The results reported above have been obtained using a very simple and straightforward unsupervised prediction procedure that counts the number of inactive SL-neighbors a target gene has. More sophisticated predictors were then used, constructed: (1) by considering additional features that describe the state of a specific gene in a given cell line based on the SL-network (for example, the average SCNA level of its SL-partners), and (2) by training on gene essentiality data to learn the important features and the classification inference procedure in what is termed a supervised manner. To this end values of 53 SL-based features for each gene-cell-line pair were extracted. These features were utilized to generate two supervised neural network classifiers of cell-line-specific gene essentiality, each one trained and tested based on a different genome-scale gene-essentiality screen (Cheung et al., 2011; Marcotte et al., 2012). A standard cross-validation prediction procedure was employed in which the test set is completely separated from the training and inner-validation involved in the generation of the neural network model. The performances of the models on the test sets resulted in ROC-curves with AUCs of 0.755 and 0.854 for the Marcotte (Marcotte et al., 2012) and Achilles (Cheung et al., 2011) data, respectively. For comparison, the nine cell lines that were tested in both screens were considered, and utilized the shRNA scores obtained in one screen to predict gene essentiality according to the other screen. Using the Achilles screen to predict gene essentiality as reported in the Marcotte screen, or vice versa, results in markedly inferior prediction performance, with AUCs of 0.663 and 0.706, respectively.

### Experimentally Validating the SL-Based Prediction of Gene Essentiality in a Breast Cancer Cell Line

To further examine the SL-based gene essentiality predictions a whole genome siRNA screen was conducted in the triple negative breast cancer cell line BT549 under normoxia and hypoxia. As BT549 was examined also in the shRNA screen of (Marcotte et al., 2012), it was possible to compare the fit between the herein presented SL-based predictions and each of the experimental screens to the fit between each of these two screens to the other. To this end the SL-based neural network predictor was trained based on the data obtained in Marcotte, after discarding the BT549 cell-line included originally in that collection. The resulting predictor was then used to predict gene essentiality in BT549, and the predictions were examined according to the results reported in (Marcotte et al., 2012). As a competing predictor the results reported in the new BT549 siRNA screen were used to predict those reported in the BT549 Marcotte screen. Remarkably, the SL-based neural network model predicts gene essentiality in BT549 significantly better than the predictions obtained using the new experimental siRNA screen conducted under normoxia or under hypoxia (an AUC of 0.842 vs. AUCs of 0.625, and 0.618, respectively). Furthermore, the performance of the SL-based predictor is further improved on a more refined set of genes that were found to be essential in BT549 according to both the previous and current screens, obtaining a very high AUC of 0.951 (FIG. 6C). Similar trends were observed when using the unsupervised SL-based predictor, and the supervised predictor trained on the Achilles shRNA data.

Underexpression of SL-Pairs is Associated with Better Prognosis in Breast Cancer

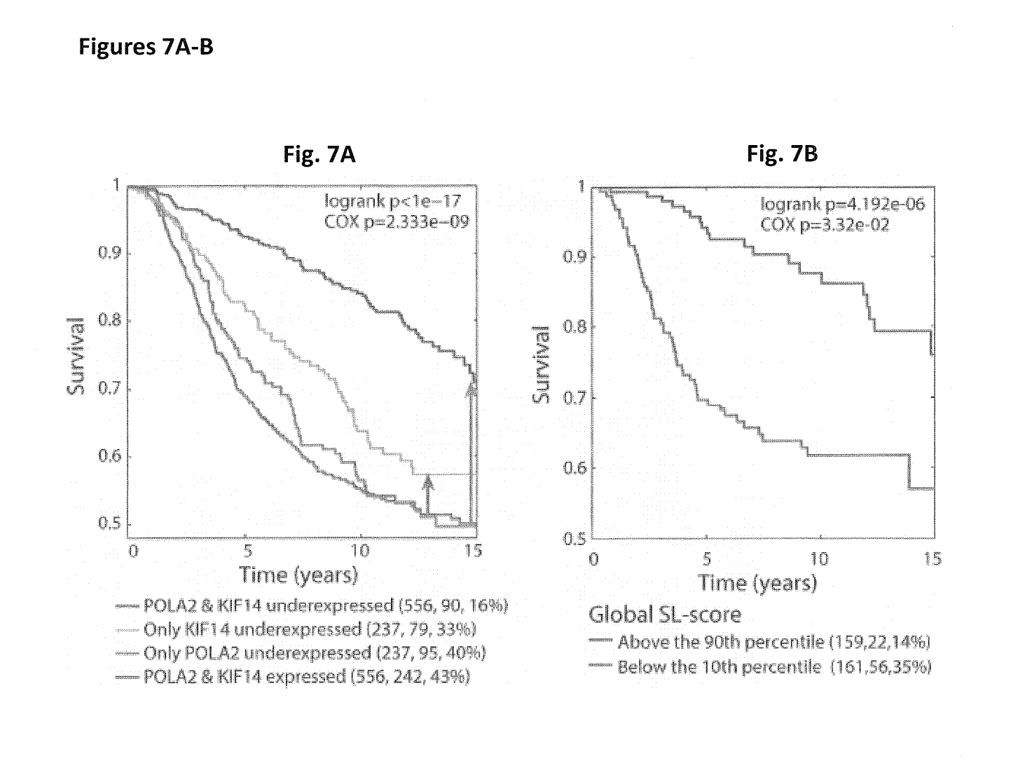

To examine the SL-network in a clinical setting gene expression and 15-year-survival data in a cohort of 1,586 breast cancer patients were analyzed (Curtis et al., 2012). It was postulated that co-underexpression of two SL-paired genes would increase tumor vulnerability, and result in better prognosis. To test this, according to each SL-pair, the patients were classified into two groups: patients whose tumors co-underexpressed the two SL-paired genes (low-group, expression of both genes is below their median levels), and patients whose tumors expressed at least one of these genes (high-group). For each SL-pair a signed Kaplan-Meier (KM)-score was computed. The higher the signed KM-score is, the better the prognosis of the low-group is compared to the high-group. Indeed, the signed KM-score of the SL-pairs are significantly higher than those of randomly selected gene-pairs (one-sided Wilcoxon rank sum p-value of 3.09e-59). It was examined if this result arises from the mere essentiality of genes in the SL-network rather than the interaction between them by repeating the analysis with (1) single genes from the SL-network, and (2) randomly selected gene-pairs involving genes from the SL-network that are not connected by SL-interactions. Reassuringly, the SL-pairs have significantly higher signed KM-scores both compared to single SL-genes and compared to random SL-network-gene-pairs (one-sided Wilcoxon rank sum p-values of 1.67e-05 and 2.00e-09, respectively). Highly significant KM-plots were obtained based on 271 SL-pairs (log rank and Cox regression p-values<0.05, following multiple hypotheses testing correction, Table 5, FIG. 7A).

Next, the patients were classified according to all the SL-pairs in the network together. For each sample a global SL-score that denotes how many of the SL-pairs it co-underexpressed was computed. As predicted, samples that co-underexpressed a high number of SL-pairs had a significantly better prognosis compared to those that co-underexpressed a low number of SL-pairs (log rank p-value of 1.482e-07, FIG. 7B). It was examined if this result is due to the mere essentiality of the SL-network genes or due to the SL-network interactions. To this end, the KM-analysis described above was repeated with 10,000 random networks consisting of genes that were found essential in breast cancer (Marcotte et al., 2012). The random networks preserve the topology of the SL-network—only the identity of the nodes is replaced by randomly selecting it from breast cancer essential genes. According to each one of these random networks the samples were divided into four classes based on the number of connected gene-pairs they co-underexpressed. Reassuringly, none of these 10,000 networks managed to separate the samples as significantly as the SL-network.

As breast cancer is a highly heterogeneous disease the utility of the global SL-scores across specific and more homogenous breast cancer groups was examined. The clinical samples were divided into separate groups according to either grade, subtype or genomic instability level (as previously defined by Bilal et al., 2013). For each group of patients, all consisting of the same subtype, grade, or genomic instability level, it was examined whether higher global SL-scores are associated with improved prognosis. This is indeed the case for all groups except one—grade 1 patients. The global SL-scores provide the most significant separation in the grade 2, normal-like subtype, and moderate genomic instability groups (log rank p-values of 8.64e-05, 1.01e-03, and 1.25e-04, respectively). As expected, the global SL-score is significantly negatively correlated with the tumor grade and genomic instability level (Spearman correlation coefficients of −0.407 and −0.267, p-values of 2.58e-62 and 2.43e-27, respectively), and highly associated with the tumor subtype (ANOVA p-value of 4.32e-101). Normal-like tumors have the highest global SL-scores while basal tumors have the lowest scores. Notably, the prognostic value of the global SL-score is significant even when accounting for the tumor grade, subtype, or genomic instability level (Cox p-values of 1.98e-04, 2.08e-08, and 2.89e-09, respectively). Lastly, the prognostic value of the global SL-scores is superior to that obtained by using genomic instability levels.

### Harnessing SDL-Interactions to Predict Drug Efficacy

The DAISY system was applied to identify all candidate SDL-pairs and a cancer SDL-network was constructed. The overlap between the SDL-interactions that were inferred based on the different datasets is significantly higher than expected by random. The network includes 3,022 genes and 3,293 SDL-interactions.

The utility of harnessing the SDL-network to predict the response of different cancer cell lines to anticancer drugs based on their genomic profiles was examined. As these drugs target mainly oncogenes, the SDL-network was chosen to predict their efficacy rather than the SL-network, which indeed yields a lower performance in this task. Two datasets of drug efficacies were utilized that were measured in a panel of cancer cell lines: (1) The Cancer Genome Project (CGP) data (Garnett et al., 2012), and (2) the Cancer Therapeutics Response Portal (CTRP) data (Basu et al., 2013). Using the SDL-network and the genomic profiles of the cancer cell lines (Barretina et al., 2012; Garnett et al., 2012), it was predicted for each drug which cell lines are sensitive and which are resistant to its administration. The prediction algorithm works in an analogous manner to the unsupervised SL-based scheme that was presented earlier for predicting gene essentiality.

The SDL-network enabled predicting the response of 593 cancer cell lines to 23 drugs, and of 241 cancer cell lines to 32 additional drugs, when utilizing the CGP and CTRP datasets to test the predictions, respectively. Overall, it was found that drugs are significantly more effective in cell lines that are predicted to be sensitive than in cell lines that are predicted to be resistant (empirical p-values of 3.525e-04 and 1.017e-04, based on the CGP and CTRP datasets, respectively).

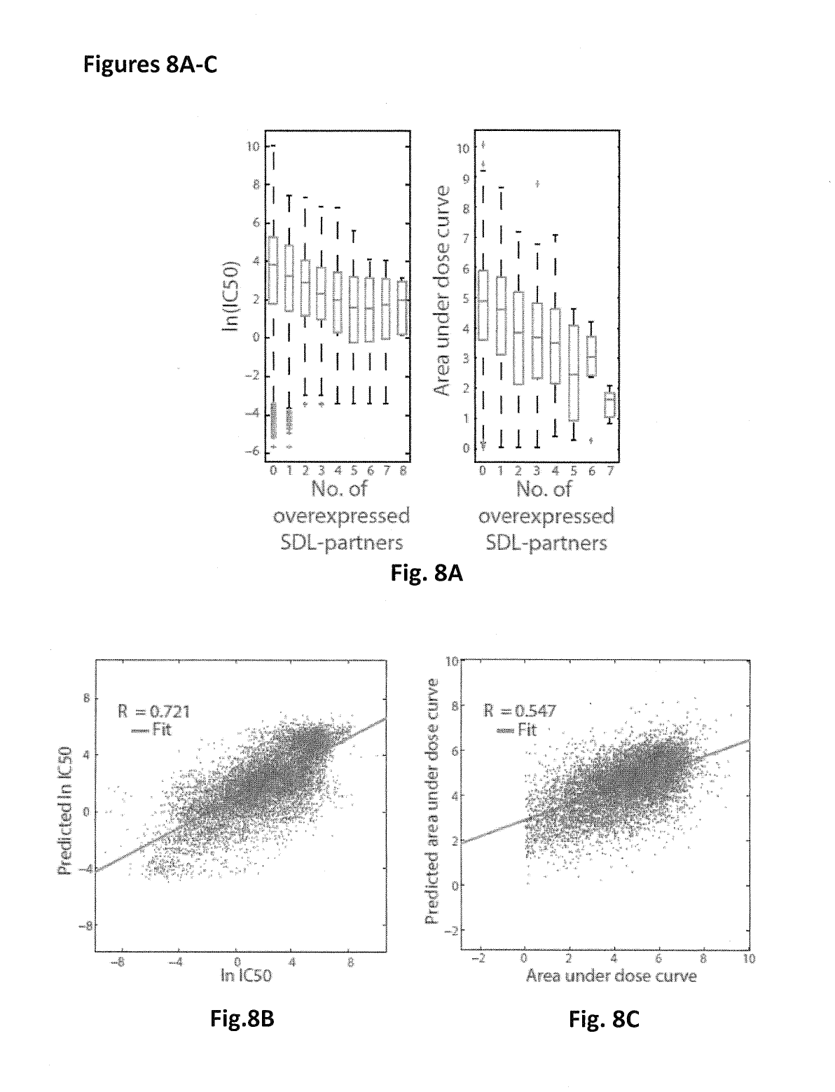

Checking the variation in the accuracy of the prediction-signal across the different drugs it was found that the more SDL-partners the drug-targets have in the SDL-network, the more accurately the SDL-network enables to predict which cell lines will be sensitive to the drug (Spearman correlation of 0.486 and 0.515, p-values of 9.29e-03 and 1.25e-03, for the CGP and CTRP datasets, respectively). Likewise, when considering only the predictions that were obtained for drugs with a sufficiently high number of SDL-interactions, the fraction of drugs that are significantly predicted increases. It was also found that the IC50 values of a drug decrease with the increase in the number of overexpressed SDL-pairs its targets have in a given cell-line (Spearman correlation of 0.85, p-value of 3.04e-03, FIG. 8A).

Focusing on the drugs that were predicted most accurately by using the SDL-network, it was further examined which SDL-interactions enable to successfully differentiate between sensitive and resistant cell lines in these cases. The SDL-network is highly predictive of the sensitivity to EGFR-inhibitors—Erlotinib, BIBW2992, and Lapatinib (Wilcoxon rank sum p-values of 2.88e-09, 1.55e-04, and 2.98e-08, respectively). It turns out that all the 17 SDL-interactions of EGFR can on their own lead to drug sensitivity predictions that significantly differentiate between cells sensitive and resistant to EGFR-inhibition (Wilcoxon rank sum p-value<0.05). One of the predicted SDL-partners of EGFR is IGFBP3, whose over-expression should accordingly induce sensitivity to drugs targeting EGFR. Reassuringly, it has been shown that IGFBP3 is lowly expressed in Gefitinib-resistant cells, and that the addition of recombinant IGFBP3 restored the ability of Gefitinib to inhibit cell growth (Guix et al., 2008).

The SDL-network is also highly predictive of the response to PARP-inhibitors (AZD-2281, ABT-888, and AG14361). Each one of the five SDL-interactions of PARP1 can, on its own, significantly differentiate between sensitive and resistant cell lines to PARP-inhibition). Interestingly, one of these interactions is with MDC1, which contains two BRCA1 C-terminal motifs and also regulates BRCA1 localization and phosphorylation in DNA damage checkpoint control (Lou et al., 2003). Indeed, BRCA1/2 are synthetically lethal with PARP1 (Lord et al., 2008).

In a manner analogous to that described herein for predicting gene essentiality, supervised neural network predictors of drug efficacies per cell line was created based on the 53 SDL-based-features. Two prediction models were trained and tested, one for the CGP dataset, and another for the CTRP dataset. The features used are similar to those utilized to predict gene essentiality based on the SL-network, this time describing drug-cell line pairs instead of gene-cell line pairs. Gene-cell features were converted to drug-cell features by mapping between drugs and their targets. With only 53 features it was managed to predict drug efficacies with Spearman correlation of 0.739 and 0.514, and p-values<1e-350, for the CGP and CTRP data, respectively (FIGS. 8B, 8C). Comparing between the supervised neural-network models and the naive, unsupervised algorithm described earlier which predicts drug response without the aid of any machine learning tools, it was reassuringly found that drugs which are predicted better based on the supervised approach are also predicted better based on the unsupervised approach (Spearman correlation of 0.571 and 0.501, p-values of 2.85e-4 and 2.93e-04, for the CGP and CTRP datasets, respectively).

The SDL-based predictors were further examined by analyzing the results of a new large pharmacological screen in which the efficacies of 126 drugs were measured across 825 cancer cell lines. The drugs utilized in the screen target overall 108 genes, 41 of which are included in the SDL-network. Based the SDL-network and the genomic profiles of these cell lines (Barretina et al., 2012) the efficacies of the drugs were predicted by using the unsupervised and supervised predictors (the latter were trained on the CTRP data). The SDL-based predictors obtained significant predictions (p-value<0.05) of drug efficacy (area-under-the-dose-curve) for 83 (65.87%) and 70 (55.6%) drugs, when applying the unsupervised or supervised approach, respectively. As previously shown based on the CGP and CTRP data, it was found again that the SDL-network is highly predictive of the response to EGFR, PARP1, BCL2, and HDAC2 inhibitors. Overall, the response to drugs targeting 28 (68.3%) and 26 (63.4%) SDL-genes is predicted in a significant manner (combined p-value<0.05), using the unsupervised or supervised approach, respectively. The prediction-signals of both approaches are strongly correlated (Spearman correlation of 0.645, p-value of 3.845e-16.

### Examining the Symmetry of Synthetic Lethal Interactions

Synthetic Lethal (SL) and Synthetic Dosage Lethal (SDL) interactions are not necessarily symmetric. Meaning, if inactivation (amplification) of gene A renders gene B essential, it does not necessarily imply that inactivation (amplification) of B renders A essential. The symmetry of SL- and SDL-interactions was examined based on the interactions inferred via DAISY. Interactions that could not have been examined in both directions were excluded from this analysis. Overall, the fraction of symmetric interactions is relatively low, and even, in some cases, less than expected if gene pairs were randomly selected.

Asymmetry may arise due to the evolutionary nature of cancer development. When genetic changes occur chronologically the perturbation of a gene induces cellular changes that affect the response to subsequent genetic perturbations, breaking the symmetry between SL- and SDL-pairs. For example, the inactivation of a tumor suppressor may relax the regulation of a certain oncogene. The cancer cells will grow to depend on this particular oncogene, a phenomenon known as “oncogene addiction” (Weinstein and Joe, 2008), and will hence be highly sensitive to its inhibition. On the other hand, it is unlikely that the loss of the oncogene will render the tumor suppressor essential.

To examine if this suggested phenomenon is manifested in the SL-network of the present invention, information of cancer-associated genes was extracted: oncogenes, tumor suppressors, cancer amplification and deletion drivers (Beroukhim et al., 2010; Chan et al., 2010; Zhao et al., 2013). Based on these gene annotations the SL-network is enriched with interactions of the form: tumor suppressor→oncogene, and deletion driver→amplification driver (hypergeometric p-values of 2.12e-04, and 2.69e-34, respectively). On the other hand, the network is not enriched for the opposite interactions: oncogene→tumor suppressor, and amplification driver→deletion driver (hypergeometric p-values of 0.689, and 1.00, respectively). These results support the hypothesis suggested above.

In addition, the complexity of cellular processes such as metabolism, regulation and signaling may also generate asymmetric interactions. For example, when considering SDL-interactions, if the over-activity of gene A generates a toxic metabolite which is detoxified by gene B, the over-activity of A will render B essential, though the other direction will not necessarily hold.

### Network Analysis and Visualization

The SL- and SDL-networks were clustered by applying the Girvan-Newman fast greedy algorithm as implemented by the GLay Cytoscape plug-in (Morris et al., 2011; Su et al., 2010). A gene-annotation enrichment analysis was performed for every network, and every network-cluster via DAVID (Huang et al., 2008, 2009). Interactive maps of networks according to the present invention are accessible through http://www.cs.tau.ac.i1/˜livnatje/SL network.cys and http://www.cs.tau.ac.i1/˜livnatje/ASL network.cys, and can be explored using the Cytoscape software (Cline et al., 2007). The maps include different gene properties and annotations, as well as alternative views that dissect the network hubs or genes with specific characteristics.

The enrichment of the SL and SDL networks with cancer-associated genes of five types was examined: (1) anticancer drug targets (Knox et al., 2011); (2) oncogenes and (3) tumor suppressors (Chan et al., 2010; Zhao et al., 2013), and cancer (4) amplification and (5) deletion drivers (Beroukhim et al., 2010). The SL and SDL networks are enriched with these cancer associated gene types, especially when considering genes with a high degree in the network.

### Harnessing the SL-Network to Assess Gene Essentiality in Cancer Cell Lines

### Robustness Analysis

To apply the SL-network for predicting gene essentiality in a cell line specific manner an approach that depends on two parameters: Deletion_{cutoff} and SLessentiality_{cutoff} was developed. The former denotes the SCNA level under which an underexpressed gene is considered inactive, and the latter denotes the number of inactive SL-partners required to deduce that a gene is essential (for further details see Experimental Procedures). This approach was applied to predicted gene essentiality based on the SL-network in 46 cancer cell lines. For these cell lines both gene expression and SCNA data were available to generate the predictions and gene essentiality data for validation (Barretina et al., 2012; Marcotte et al., 2012).

In addition to the results obtained with a Deletion_{cutoff} of −0.1 and an SLessentiality_{cuttoff} of 1. The network performances across a broad range of parameters were examined. The Deletion_{cutoff} and SLessentiality_{cuttoff} parameters were set to 10 different values each, ranging from −0.1 to −1, and from 1-10, respectively. In each setting the predictive signal of the network was computed by the four empirical p-values described in the Experimental Procedures. The network performances is highly robust across a fairly broad range of definitions. However, the more stringent the gene loss and essentiality definitions are, the less predictions could be made for more genetically stable cell lines. Likewise, genes that have a number of SL-partners that is below the SLessentiality_{cuttoff} parameter could not have been predicted as essential in any cell line, regardless of the genomic profiles of the cell lines.

The SCNA level of a gene is the observed vs. expected number of copies it has in a given sample, on a log₂ scale. Hence, if the reference state has two copies of a given gene, a SCNA level of −1 is equivalent to a heterozygous loss of a gene, meaning, one copy. It should be noted, that SCNA data is measured at the population-level, and hence contains the average SCNA level of a given gene in a population of cells. If the sample is contaminated with normal cells, the copy number of the cancer cells will be more extreme, that is, the SCNA level of the cancer cells will be higher or lower if the measured SCNA level is positive or negative, respectively. A heterogeneous population of cancer cells that contains several clones will also add noise to the data. Nonetheless, it is assured that there is at least one cancer clone that has an integer copy-number which is at least as low as the measured copy-number.

Ideally one would like to set Deletion_{cutoff} such that only genes with homozygous deletions will be defined as deleted. A full deletion of a gene is a rare event—in 78.4% of the cancer SCNA profiles that were analyzed there is not a single gene with a SCNA level less than −1 (Beroukhim et al., 2010). Therefore, several, more moderate, definitions of gene loss (setting the Deletion_{cutoff} to 10 different values ranging from −0.1 to −1) were tested. To ensure that the low SCNA level is also observed in the levels of the gene, a gene was defined as inactive only if it was also underexpressed (with a low mRNA levels) in the cancer cell line, as explained in Experimental Procedures. As gene deletion was defined more permissively, one (partially) deleted SL-partner may not be sufficient to render a gene essential. Hence, more stringent definitions of gene essentiality were examined (setting the SLessentiality_{cuttoff} parameter to 10 different values, ranging from 1-10).

### The Prediction-Signal and Genetic Instability

It was postulated that the SL-network will obtain more accurate gene-essentiality-predictions for cell lines with a higher number of inactive genes as compared to cell lines with lower number of inactive genes. In cell lines with many inactive genes it is more likely that the essentiality of more genes will arise due to synthetic lethality, rather than due to other causes which are not related to synthetic lethality, and hence cannot be captured by the SL-network. To examine this hypothesis, for each cell line the fraction of its inactive genes was computed. The Spearman correlation across all cell lines between this measure and the prediction-signal that was obtained for each cancer cell line was then computed.

The prediction-signal is defined in two ways: (1) the −log(p-value) of the hypergeometric test that denotes per cell line if the genes that were predicted as essential in it are enriched with essential genes, and (2) the −log(p-value) of the Wilcoxon rank sum test denoting if the gene essentiality (zGARP) score of the predicted essential genes is significantly lower compared to the score of other genes in the cell line, according to (Marcotte et al., 2012). The reference set for comparison for the two definitions of predictions signal was either all genes or only the genes in the network, resulting in four prediction-signal measures.

A significant correlation between the fractions of inactive genes and the prediction-signals was found, showing that the more genes the cell line has lost, the better the SL-network predicts its essential genes. This correlation increases when applying more stringent definitions of gene loss (Deletion_{cutoff}) and essentiality (SLessentiality_{cuttoff}).

### Comparison to the Yeast-Derived SL-Network

The gene essentiality predictions were repeated with the yeast-derived SL-network, originally termed the inferred Human SL Network (iHSLN) (Conde-Pueyo et al., 2009). The predictions were evaluated as described in the Experimental Procedures. The results obtained by the SL-network were significantly superior to those obtained by the iHSLN.

### The SDL-Network and its Properties

DAISY was applied to identify all candidate SDL-pairs to construct an SDL-network. The overlap between the SDL-interactions that were inferred based on the different datasets is significantly high, demonstrating the predictions' consistency. The SDL-network includes 3,022 genes and 3,293 SDL-interactions. The SDL-network and the SL-network share 961 genes, with 3 overlapping interactions. Similar to the SL-network, the SDL-network also displays scale-free like characteristics. It is enriched with cancer associated genes and with 144 Gene Ontology (GO) annotations. The top GO annotations are: RNA processing and splicing, transcription, cell cycle, mitotic cell cycle, mRNA metabolic process, and DNA metabolic process.

### Robustness Analysis of Drug Predictions

The SDL-network was utilized to predict drug-efficacy in an unsupervised manner. The prediction is based on two parameters: Overexpression_{cutoff} and SDLessentiality_{cutoff} (see Experimental Procedures). The drug efficacy predictions were repeated with different definitions of gene overexpression (Overexpression_{cutoff}) and gene essentiality (SDLessentiality_{cutoff}), ranging from 50-90 and 1-5, respectively. As explained the Experimental Procedures, for each drug its efficacy in the cell lines that were predicted to be sensitive and in the cell lines that were predicted to be resistant to its administration (one-sided Wilcoxon rank sum test) were compared. The efficacy is represented by the IC50-values, or area-under-dose-curve, when testing the predictions based on the Cancer Genome Project (CGP) (Garnett et al., 2012) and the Cancer Therapeutics Response Portal (CTRP) data (Basu et al., 2013), respectively. An empirical p-value that denotes the significance of the predictions obtained across all the different drugs was then computed. The prediction-signal, as shown by these empirical p-values, is highly robust across a fairly broad range of definitions. However, when employing more stringent gene essentiality definition (SDLessentiality_{cutoff}) the efficacy of drugs whose targets have a low number of SDL-interactions could not be predicted. It was found that the more SDL-partners the drug-target has, the better the SDL-network enables to accurately differentiate between the cell lines that are sensitive and the cell lines that are resistant to its administration.

### Predicting Drug-Response Based on SL-Interactions

The SL-network does not enable to accurately predict the response of cancer cell lines to the administration of different anticancer drugs. This may possibly be due to the fact that these drugs target oncogenes, whose essentiality is mainly dictated by other types of genetic interactions, as SDL-interactions. Supporting this claim, the SL-network predicts best the response to a PARP1 inhibitor (ABT-888, one-sided Wilcoxon rank sum p-value 0.046, CGP data), which is one of the few anticancer drug that rely on synthetic lethality. For comparison, as PARP1 is synthetically lethal with BRCA1/2 (Lord et al., 2008; Turner et al., 2008), the GDC cell lines were divided according to their BRCA1/2 mutation-status and it was predicted that the mutated cell lines will be sensitive to PARP-inhibition. The IC50 values of ABT-888 in the predicted sensitive and in the predicted resistant cell lines were compared via a one-sided Wilcoxon rank sum, and obtained p-value of 0.889. The SCNA and mRNA levels of the BRCA genes were also used to deduce which cell lines have an inactive form of BRCA1/2. When predicting these cell lines as sensitive a one-sided Wilcoxon rank sum p-value 0.902 was obtained.

Exemplary SL and SDL networks identified by the systems and methods disclosed herein.

TABLE 1
SL network which comprises the gene pairs listed.
When gene A is deleted gene B is essential

| Gene A | Gene B |
|---|---|
| ACAP1 | DEF6 |
| ACAP1 | GIMAP1 |
| ACAP1 | MAP4K1 |
| ACAP1 | SEMA4A |
| ACD | SMARCC2 |
| ACD | SNRPA |
| ACIN1 | AZI1 |
| ACIN1 | BAZ1B |
| ACIN1 | DCAF16 |
| ACIN1 | GGA3 |
| ACIN1 | UBE2O |
| ACP1 | GLUD2 |
| ACP1 | LIG4 |
| ACP1 | MAPRE1 |
| ACP1 | RAB23 |
| ACP1 | ZBTB6 |
| ACTN1 | PROCR |
| ACTN1 | S100A11 |
| ACTN1 | SERPINB6 |
| ACTN1 | ZYX |
| ACVR1 | CALU |
| ADAM10 | ATP6V1A |
| ADAM9 | ANXA4 |
| ADAM9 | NPC2 |
| ADAM9 | RAB11FIP5 |
| ADAM9 | RHOC |
| ADAMTS8 | APOA2 |
| ADAT2 | POLR1B |
| ADAT2 | RPIA |
| ADM | ANXA2 |
| ADM | EPAS1 |
| ADM | PTRF |
| ADORA2B | EMP1 |
| ADRA1A | TACR1 |
| ADRA1A | THPO |
| ADRB1 | LRTM1 |
| AFAP1 | FAM127A |
| AFAP1 | SNX21 |
| AGA | CTBS |
| AGGF1 | DLD |
| AGGF1 | TPRKB |
| AGPAT5 | HNRNPA3 |
| AHNAK2 | KIRREL |
| AHNAK2 | PPP1R13L |
| AHNAK2 | RIN2 |
| AIM1L | ENTPD2 |
| AIM1L | JUP |
| AIM1L | PRRG2 |
| AIMP1 | EXOC5 |
| AIMP1 | RNF146 |
| AIMP1 | UCHL5 |
| AKAP4 | BMP8A |
| AKR1C2 | UGT1A7 |
| ALDH18A1 | CCT2 |
| ALDH18A1 | DLAT |
| ALDH18A1 | DNAJB6 |
| ALDH18A1 | MTFR1 |
| ALDH18A1 | TM9SF2 |
| ALDH1A3 | TINAGL1 |
| ALPI | ZNF749 |
| ALPK1 | CAMKK2 |
| ALPK1 | KCNJ5 |
| ALPK1 | PILRA |
| ALPK1 | ZNF692 |
| AMZ2 | HRSP12 |
| AMZ2 | HSPA8 |
| ANAPC10 | C19orf2 |
| ANAPC10 | HBS1L |
| ANAPC10 | UGP2 |
| ANKFY1 | ARF1 |
| ANKFY1 | C19orf2 |
| ANKFY1 | HSPA8 |
| ANKFY1 | LMBRD1 |
| ANKFY1 | MED17 |
| ANKFY1 | MLLT10 |
| ANKFY1 | SDHB |
| ANKFY1 | SDHC |
| ANKRD1 | AXL |
| ANKRD22 | MAPK13 |
| ANKRD22 | SERPINB5 |
| ANKRD22 | SLC37A1 |
| ANP32A | CNOT10 |
| ANP32A | NUP160 |
| ANP32A | ZNF124 |
| ANP32B | HNRNPA1 |
| ANXA1 | LMNA |
| ANXA1 | RASAL2 |
| ANXA1 | SERPINH1 |
| ANXA2 | ACTN4 |
| ANXA2 | CFB |
| ANXA2 | ELOVL1 |
| ANXA2P1 | AHNAK |
| ANXA2P1 | ELOVL1 |
| ANXA2P1 | LIMA1 |
| ANXA2P1 | PROCR |
| ANXA2P1 | RAB11FIP5 |
| ANXA2P2 | PERP |
| ANXA2P2 | PLCD3 |
| ANXA2P2 | TGM2 |
| ANXA5 | BNC2 |
| ANXA5 | NAV3 |
| ANXA5 | OSMR |
| ANXA7 | PEX13 |
| AP3B1 | HSPA8 |
| AP3B1 | LMAN1 |
| AP3B1 | PSMA3 |
| AP3B1 | RPAP3 |
| AP3B1 | TMED2 |
| API5 | DLD |
| API5 | GIN1 |
| API5 | ILF2 |
| API5 | MATR3 |
| API5 | TRIM23 |
| API5 | VAMP3 |
| API5 | YAF2 |
| API5 | ZFYVE21 |
| API5 | ZNF780A |
| APOL1 | CFB |
| APOL3 | OAS2 |
| APPL2 | ARFGEF1 |
| ARF4 | ATP6V1C1 |
| ARF4 | COPB2 |
| ARF4 | LMNA |
| ARF4 | MCL1 |
| ARF4 | RHEB |
| ARFGEF1 | ATP5F1 |
| ARFGEF1 | GTF3C3 |
| ARFGEF2 | NRBF2 |
| ARGLU1 | FUBP1 |
| ARHGAP11A | NCAPH |
| ARHGAP11A | SMC4 |
| ARHGAP19 | CORO1A |
| ARHGAP19 | LIG1 |
| ARHGAP19 | MSL2 |
| ARHGAP19 | NUDT21 |
| ARHGAP19 | SFPQ |
| ARHGAP19 | SNRPD1 |
| ARHGAP29 | CRIM1 |
| ARHGAP29 | TNFAIP1 |
| ARHGAP33 | PKMYT1 |
| ARID1A | CTCF |
| ARID1A | SF1 |
| ARID1A | TROAP |
| ARID1B | BPTF |
| ARMC1 | EXOC5 |
| ARMC6 | NHP2 |
| ARMC6 | PRPF19 |
| ARSB | LEPRE1 |
| ASF1A | MATR3 |
| ASF1B | BRCA1 |
| ASPH | SEMA3C |
| ATAD2B | NASP |
| ATAD5 | CDC7 |
| ATAD5 | CENPF |
| ATAD5 | FANCM |
| ATAD5 | FUBP1 |
| ATAD5 | LIN9 |
| ATAD5 | MCM2 |
| ATAD5 | MYBL2 |
| ATAD5 | NASP |
| ATAD5 | PNN |
| ATAD5 | POLE2 |
| ATAD5 | RAD54L |
| ATAD5 | RFC4 |
| ATAD5 | SFPQ |
| ATAD5 | SRRT |
| ATAD5 | TOPBP1 |
| ATAD5 | WDHD1 |
| ATG2A | SOLH |
| ATG2A | TBL3 |
| ATG2A | ZC3H7B |
| ATG5 | DERL1 |
| ATG5 | DNAJB6 |
| ATG5 | ITFG1 |
| ATG5 | MMADHC |
| ATG5 | UBE2H |
| ATP2C2 | SPINT2 |
| ATP5B | AIFM1 |
| ATP5C1 | NMD3 |
| ATP6AP2 | DCTN4 |
| ATP6AP2 | IL13RA1 |
| ATP6AP2 | LAMP2 |
| ATP6AP2 | UGP2 |
| ATP6V0E1 | CSTB |
| ATP6V1C1 | CUL4B |
| ATP6V1C1 | SDHC |
| AURKB | CKS1B |
| AURKB | ERCC6L |
| AURKB | SNRPA |
| AURKB | TK1 |
| AVPI1 | ADAM9 |
| AVPI1 | CST3 |
| AVPI1 | CTSB |
| AVPI1 | RIPK4 |
| AVPI1 | SGMS2 |
| B3GNT2 | RANBP9 |
| B4GALT1 | EPAS1 |
| BAG3 | ADAM9 |
| BAG3 | CPA4 |
| BAG3 | EGFR |
| BAG3 | LARP6 |
| BAG3 | LMNA |
| BAG3 | S100A11 |
| BAG3 | TNFRSF1A |
| BAIAP2L1 | ARHGEF16 |
| BAIAP2L1 | FRK |
| BAIAP2L1 | RIPK4 |
| BARD1 | SNRPA |
| BAZ1B | E2F1 |
| BAZ1B | H1FX |
| BCAR3 | ARSJ |
| BCAR3 | GPX8 |
| BCAR3 | LARP6 |
| BCAR3 | S100A13 |
| BCAR3 | S100A2 |
| BCAR3 | SMAD3 |
| BCAR3 | TNFAIP1 |
| BCL9L | IGFBP6 |
| BCL9L | S100A11 |
| BCLAF1 | HNRNPA3 |
| BDNF | GNG11 |
| BEND3 | LBR |
| BIN2 | PILRA |
| BLK | IKZF1 |
| BLM | CCDC138 |
| BLM | MCM2 |
| BLM | MCM6 |
| BLM | RFC4 |
| BLM | TIMELESS |
| BLM | TOPBP1 |
| BLMH | XRCC5 |
| BMP1 | SERPINH1 |
| BMP8A | KCNH6 |
| BRCA1 | EXO1 |
| BRCA1 | FEN1 |
| BRCA2 | DLGAP5 |
| BRCA2 | STIL |
| BRD2 | ZNF611 |
| BRD4 | CCNT1 |
| BRD4 | GGA3 |
| BRD4 | TNK2 |
| BRF1 | DNASE1L2 |
| BRIP1 | DTL |
| BRIP1 | FH |
| BRIP1 | GDAP1 |
| BRIP1 | POLA1 |
| BRIP1 | PSMC3 |
| BRPF1 | BRD2 |
| BRPF1 | KDM2B |
| BSPRY | C2orf15 |
| BSPRY | FA2H |
| BSPRY | GRHL1 |
| BTBD7 | POLH |
| BTG2 | SESN1 |
| BUB1B | AURKA |
| BUB1B | CENPI |
| BUB1B | CKAP5 |
| BUB1B | DSCC1 |
| BUB1B | MDC1 |
| BUB1B | SKP2 |
| BUD13 | MCM4 |
| BYSL | CCT2 |
| C10orf2 | PHB2 |
| C10orf35 | KIAA0895 |
| C10orf47 | ARHGEF5 |
| C10orf47 | DSG2 |
| C11orf58 | ARPC5 |
| C11orf58 | CD46 |
| C11orf58 | CDC5L |
| C11orf58 | DLD |
| C11orf58 | DNAJC10 |
| C11orf58 | HRSP12 |
| C11orf58 | MAT2A |
| C11orf58 | MSH2 |
| C11orf58 | MSH6 |
| C11orf58 | NUDT21 |
| C11orf58 | PDCD5 |
| C11orf58 | PNO1 |
| C11orf58 | POLR2K |
| C11orf58 | PPP1R2 |
| C11orf58 | PSMD12 |
| C11orf58 | SGPP1 |
| C11orf58 | TPRKB |
| C11orf58 | UGP2 |
| C11orf58 | ZNF780A |
| C11orf73 | PIK3CA |
| C11orf73 | PSMD10 |
| C12orf47 | RMND5A |
| C15orf42 | TOPBP1 |
| C15orf52 | ACTN4 |
| C17orf48 | C19orf2 |
| C17orf48 | CCT2 |
| C17orf48 | DLD |
| C17orf48 | MED17 |
| C17orf48 | MLLT10 |
| C17orf48 | SENP2 |
| C17orf48 | TFB2M |
| C17orf48 | TMED2 |
| C17orf48 | ZNF227 |
| C17orf62 | GMIP |
| C17orf70 | TBC1D10B |
| C17orf70 | ZBTB17 |
| C17orf70 | ZNF335 |
| C19orf10 | P4HB |
| C19orf21 | EPCAM |
| C19orf66 | HLA-E |
| C1orf112 | CDC25C |
| C1orf135 | MYBL2 |
| C1orf135 | PKMYT1 |
| C1orf200 | RPL13AP17 |
| C20orf202 | DEFB118 |
| C20orf30 | B3GNT2 |
| C20orf30 | C5orf44 |
| C20orf30 | COPS8 |
| C20orf30 | HNRNPF |
| C20orf30 | IL20RB |
| C20orf30 | LIPT1 |
| C20orf30 | MINPP1 |
| C20orf30 | MRPL19 |
| C20orf30 | PRKRA |
| C20orf30 | PSMC6 |
| C20orf30 | RAD17 |
| C20orf30 | SMU1 |
| C20orf30 | UQCRC2 |
| C2orf44 | NASP |
| C4orf21 | CDC7 |
| C4orf21 | GEN1 |
| C4orf21 | MCM2 |
| C4orf29 | WDR33 |
| C4orf46 | HNRNPH1 |
| C6orf162 | MATR3 |
| C6orf162 | RBM12B |
| C6orf25 | NMUR1 |
| C9orf46 | ARPC5 |
| C9orf91 | SLC1A4 |
| CA4 | CELA2B |
| CABIN1 | NCOR2 |
| CABIN1 | PPP1R10 |
| CABIN1 | RARA |
| CALU | CD276 |
| CALU | CD63 |
| CALU | EXOC5 |
| CALU | FNDC3B |
| CALU | MAP1LC3B |
| CALU | SENP2 |
| CALU | ZCCHC24 |
| CALY | EMX1 |
| CAP2 | LAMB2 |
| CAPN7 | C1orf56 |
| CAPN7 | H3F3C |
| CAPN7 | MSH6 |
| CAPN7 | NFE2L2 |
| CAPN7 | PIP5K1A |
| CAPN7 | POGK |
| CAPN7 | SRP9 |
| CAPRIN1 | ADNP |
| CAPRIN1 | ARF1 |
| CAPRIN1 | AZIN1 |
| CAPRIN1 | C1orf56 |
| CAPRIN1 | CANX |
| CAPRIN1 | CCT2 |
| CAPRIN1 | CUL4B |
| CAPRIN1 | DLD |
| CAPRIN1 | FH |
| CAPRIN1 | GLRX3 |
| CAPRIN1 | GLUD1 |
| CAPRIN1 | GLUD2 |
| CAPRIN1 | HRSP12 |
| CAPRIN1 | NFE2L2 |
| CAPRIN1 | PPP1R2 |
| CAPRIN1 | PRDX3 |
| CAPRIN1 | PTGES3 |
| CAPRIN1 | SRP9 |
| CAPRIN1 | UGP2 |
| CAPRIN1 | YAF2 |
| CAPRIN1 | ZFYVE21 |
| CAPRIN1 | ZNF780A |
| CARD10 | AMIGO2 |
| CARD10 | DHRS3 |
| CARD10 | GPRC5A |
| CARD10 | TNFRSF1A |
| CARD10 | TSPAN1 |
| CARM1 | GGA3 |
| CASP8 | PSMB8 |
| CAST | AHNAK |
| CAST | CAPN2 |
| CAST | LAMB3 |
| CAST | S100A13 |
| CBFB | SFPQ |
| CC2D1A | KCNH6 |
| CC2D1A | SFTPB |
| CCAR1 | SF3B1 |
| CCAR1 | TOPBP1 |
| CCBE1 | SERPINE1 |
| CCDC130 | GPR44 |
| CCDC130 | SMARCC2 |
| CCDC138 | DSCC1 |
| CCDC76 | CCT2 |
| CCDC88C | EPB41 |
| CCDC88C | PDIK1L |
| CCDC88C | RBM38 |
| CCL19 | TSKS |
| CCNA2 | MCM2 |
| CCNA2 | MYBL2 |
| CCNA2 | NCAPG2 |
| CCNA2 | POLA2 |
| CCNA2 | TOPBP1 |
| CCNB1 | CDKN3 |
| CCNC | ANAPC10 |
| CCNC | HRSP12 |
| CCNC | MRPL3 |
| CCNC | ZFAND1 |
| CCNF | EHMT2 |
| CCNG2 | B3GNT2 |
| CCNG2 | PIK3CA |
| CCNL1 | CNOT8 |
| CCNT1 | CNOT3 |
| CCR7 | CD52 |
| CCT8 | POLR1B |
| CD109 | S100A3 |
| CD151 | COL4A2 |
| CD151 | MT2A |
| CD163 | GHRHR |
| CD164 | API5 |
| CD226 | THPO |
| CD276 | SERPINH1 |
| CD46 | PRKAA1 |
| CD52 | ADRB1 |
| CD63 | GDF15 |
| CD63 | NBL1 |
| CD63 | SLC38A6 |
| CDA | LAMB3 |
| CDADC1 | FCGR3A |
| CDC20 | CEP152 |
| CDC20 | CEP55 |
| CDC20 | KIF14 |
| CDC20 | PKMYT1 |
| CDC20 | TIMELESS |
| CDC20 | TK1 |
| CDC25A | CPSF6 |
| CDC25A | LBR |
| CDC25A | MSH6 |
| CDC25A | PTMA |
| CDC25A | RMND5A |
| CDC25C | CCNA2 |
| CDC25C | MCM7 |
| CDC25C | NDC80 |
| CDC25C | PLK4 |
| CDC42BPB | GIPC1 |
| CDC42BPB | ZNF358 |
| CDC42EP1 | AHNAK |
| CDC42EP1 | PRSS23 |
| CDC42EP1 | TM4SF1 |
| CDC45 | HIRIP3 |
| CDC45 | MYBL2 |
| CDC45 | SMC2 |
| CDC45 | TRIP13 |
| CDC45 | UNG |
| CDC5L | API5 |
| CDC5L | CPNE1 |
| CDC5L | HNRNPH1 |
| CDC5L | PSMC3 |
| CDC6 | CCNE2 |
| CDC6 | CDCA8 |
| CDC6 | CHAF1A |
| CDC6 | KIF2C |
| CDC6 | PCNA |
| CDC6 | STIL |
| CDC7 | CCNF |
| CDC7 | CENPA |
| CDC7 | CENPF |
| CDC7 | HNRNPA1 |
| CDC7 | MYBL2 |
| CDC7 | POLD3 |
| CDC7 | RAD51AP1 |
| CDC7 | RFC2 |
| CDC7 | SENP1 |
| CDC7 | TOPBP1 |
| CDCA2 | INCENP |
| CDCA2 | SPAG5 |
| CDCA3 | RAD51 |
| CDCA7 | ARHGAP19 |
| CDCA8 | PKMYT1 |
| CDH1 | CGN |
| CDH1 | CRB3 |
| CDH1 | EXPH5 |
| CDH1 | PLXNB1 |
| CDH1 | SH2D3A |
| CDH1 | SOX13 |
| CDH2 | DPYSL3 |
| CDH3 | CGN |
| CDK1 | MAD2L1 |
| CDK1 | SNRPA1 |
| CDK1 | STIL |
| CDK7 | ITCH |
| CDS1 | DMKN |
| CDS1 | FXYD3 |
| CDS1 | GPRC5A |
| CDS1 | MAPK13 |
| CDS1 | PLS1 |
| CDS1 | PTK6 |
| CDS1 | STYK1 |
| CDT1 | CNOT3 |
| CDT1 | EXOSC5 |
| CECR5 | POLR1B |
| CELA2B | GPR32 |
| CELF1 | NRF1 |
| CENPA | FEN1 |
| CENPA | RFC5 |
| CENPE | RAD51AP1 |
| CENPE | TOPBP1 |
| CENPH | NUF2 |
| CENPJ | POLQ |
| CENPM | MYBL2 |
| CENPM | NUP188 |
| CENPM | PCNA |
| CENPM | STIL |
| CENPO | FEN1 |
| CEP152 | CENPF |
| CEP152 | DTL |
| CEP152 | R3HDM1 |
| CEP152 | RBM15 |
| CEP152 | TOPBP1 |
| CEP152 | ZNF669 |
| CEP55 | ECT2 |
| CEP55 | SPAG5 |
| CEP78 | CDK1 |
| CEP78 | CENPO |
| CEP78 | KIFC1 |
| CETN3 | CLDND1 |
| CGRRF1 | PSMD12 |
| CHAC1 | CARS |
| CHAC1 | YARS |
| CHAF1A | ANAPC5 |
| CHAF1A | CPSF6 |
| CHAF1A | POLE2 |
| CHAF1A | RAD51AP1 |
| CHAF1A | RBM14 |
| CHAF1A | TRMT5 |
| CHAF1B | FEN1 |
| CHAF1B | INCENP |
| CHAF1B | SMC2 |
| CHAF1B | SMC4 |
| CHAF1B | TPX2 |
| CHAF1B | WDHD1 |
| CHDH | EPCAM |
| CHEK1 | KNTC1 |
| CHEK1 | SMC2 |
| CHEK1 | TMEM194A |
| CHMP1A | CORO1B |
| CHMP1B | UBE2H |
| CHMP4C | DSG2 |
| CKAP2 | DEK |
| CKAP2 | DLGAP5 |
| CKS2 | KIF14 |
| CLASP2 | CPSF6 |
| CLIC3 | S100A16 |
| CLINT1 | EXOC5 |
| CLINT1 | RNF146 |
| CLIP4 | PLAT |
| CLSPN | SMC2 |
| CMAS | GIN1 |
| CMTM4 | DSC2 |
| CMTM4 | EXPH5 |
| CMTM4 | TMEM144 |
| CNBP | BACH1 |
| CNBP | SNX2 |
| CNBP | TRIM23 |
| CNN3 | ANXA5 |
| CNN3 | PDGFC |
| CNNM4 | MARVELD2 |
| CNOT6L | MSL2 |
| CNOT8 | RAB1A |
| CNR2 | HIPK4 |
| CNR2 | PTGIR |
| COIL | RBM12 |
| COL12A1 | FSTL1 |
| COL4A2 | ANTXR1 |
| COL4A2 | KIRREL |
| COMMD10 | UCHL5 |
| COMMD8 | MMADHC |
| COMMD8 | SOAT1 |
| COPS2 | TBL1XR1 |
| COPS5 | RAB1A |
| CORO2A | F11R |
| CPEB3 | PDIK1L |
| CPSF3 | PPIH |
| CPSF6 | CDC7 |
| CPSF6 | FUS |
| CPSF6 | HNRNPR |
| CPSF6 | RAD54L |
| CRB3 | EVPL |
| CRB3 | SSH3 |
| CREB1 | SPTLC1 |
| CREB3 | CYR61 |
| CREB3 | TPM1 |
| CREBZF | IQCB1 |
| CREBZF | POU2F1 |
| CREBZF | PUM2 |
| CRISP1 | MSTN |
| CRISP1 | NCR1 |
| CROCC | DNASE1L2 |
| CROCC | RHOT2 |
| CRTAP | MSRB3 |
| CRTAP | PTRF |
| CRY2 | SMARCC2 |
| CSK | MAST3 |
| CSNK1G2 | CACNA1G |
| CSNK1G2 | CPSF1 |
| CSNK1G2 | RBM14 |
| CSNK1G2 | SMARCC2 |
| CSNK1G2 | TRIM28 |
| CTCF | LUC7L2 |
| CTCF | MATR3 |
| CTCF | NASP |
| CTDSPL2 | MCM2 |
| CTDSPL2 | SFPQ |
| CTSA | NEU1 |
| CTSB | CAV1 |
| CTSB | IGFBP6 |
| CTSB | LMNA |
| CTSB | PTPN14 |
| CTSB | S100A11 |
| CTSB | SERPINH1 |
| CTSD | EPHX1 |
| CTSD | TNFRSF1A |
| CTTNBP2NL | ANXA2 |
| CTTNBP2NL | LARP6 |
| CUL1 | DCTN4 |
| CUL1 | RAB23 |
| CUL1 | TBL1XR1 |
| CWF19L1 | CCT2 |
| CXCL1 | LTBR |
| CXCL13 | SIGLEC8 |
| CXCL16 | RAB25 |
| CXCL2 | SDC4 |
| CXCR6 | P2RX2 |
| CXXC1 | EDC4 |
| CXXC1 | SMARCC2 |
| CXorf21 | SCNN1D |
| CXorf21 | SNRPA |
| CYB5R4 | TBL1XR1 |
| CYR61 | CAPN2 |
| CYR61 | EPAS1 |
| CYR61 | LATS2 |
| CYR61 | PARVA |
| CYR61 | RUSC2 |
| CYTH3 | THBS1 |
| CYTH4 | ABI3 |
| CYTH4 | TNFAIP8L2 |
| DAG1 | SPR |
| DAPP1 | SEMA4A |
| DAZAP1 | LBR |
| DAZAP1 | PDSS1 |
| DAZAP1 | RMND5A |
| DAZAP2 | B3GNT1 |
| DBF4 | USP1 |
| DCAF12 | CPSF6 |
| DCAF12 | RMND5A |
| DCAF15 | GRK6 |
| DCAF15 | POLR1B |
| DCK | ESPL1 |
| DCK | NRF1 |
| DCK | SMC3 |
| DCK | TAF5 |
| DCTN6 | C20orf30 |
| DDOST | P4HB |
| DDX1 | NCBP1 |
| DDX11 | RECQL4 |
| DDX21 | TMEM48 |
| DDX28 | TARS2 |
| DDX3X | RAD23B |
| DDX3X | ZNF780A |
| DDX49 | U2AF2 |
| DDX5 | ARFGEF1 |
| DDX5 | CNBP |
| DDX6 | DCTN1 |
| DDX6 | SMG7 |
| DDX6 | ZC3H7B |
| DDX60 | HLA-B |
| DDX60L | PARP14 |
| DEK | HMGN1 |
| DEPDC1 | CCNB2 |
| DEPDC1 | MELK |
| DEPDC1 | RAD51AP1 |
| DGCR8 | ANAPC2 |
| DHPS | AIP |
| DHTKD1 | XRCC2 |
| DHX15 | MRPL3 |
| DHX15 | NDUFAF4 |
| DHX15 | PIK3CA |
| DHX15 | RAD23B |
| DHX30 | COPS7B |
| DHX30 | ZNF668 |
| DHX32 | IL13RA1 |
| DHX32 | S100A11 |
| DHX9 | ATAD5 |
| DHX9 | NME1 |
| DIAPH3 | HRH4 |
| DIP2A | CRY2 |
| DIP2A | DCTN1 |
| DIP2A | MAP3K3 |
| DIP2A | SFTPB |
| DIP2A | SNAPC4 |
| DIP2A | ZNF771 |
| DKK3 | CAV2 |
| DKK3 | CTGF |
| DLAT | ATP6V1A |
| DLAT | CD46 |
| DLAT | CNOT8 |
| DLAT | DCTN4 |
| DLAT | HSPD1 |
| DLAT | MRPL13 |
| DLAT | POLR2K |
| DLAT | SSBP1 |
| DLD | IARS2 |
| DLD | MRPS28 |
| DLEC1 | GLP1R |
| DLG1 | GLRX3 |
| DLG5 | CRABP2 |
| DLGAP5 | CENPF |
| DLGAP5 | MCM6 |
| DLGAP5 | MYBL2 |
| DLGAP5 | NUDT1 |
| DLGAP5 | TUBA3D |
| DMP1 | CSH1 |
| DMP1 | MLL2 |
| DMP1 | POLH |
| DMP1 | ROS1 |
| DMP1 | XYLB |
| DMTF1 | CCDC76 |
| DNA2 | EZH2 |
| DNA2 | ILF3 |
| DNA2 | PCNA |
| DNA2 | ZNF107 |
| DNAH1 | NCKAP1L |
| DNAJA2 | CD46 |
| DNAJA2 | DLD |
| DNAJA2 | HNRNPC |
| DNAJB4 | PTRF |
| DNAJB6 | DLG1 |
| DNAJB6 | MED17 |
| DNAJB6 | TBL1XR1 |
| DNM1L | PSMA3 |
| DNM2 | FAM193B |
| DNMT1 | NFATC3 |
| DNMT1 | SMARCB1 |
| DOCK1 | ADAM9 |
| DOCK1 | CTGF |
| DOCK1 | SNX21 |
| DOLK | CLPTM1 |
| DONSON | RFC4 |
| DONSON | TPX2 |
| DOT1L | RBM14 |
| DSCR3 | EXOC5 |
| DSCR3 | NMD3 |
| DSG2 | KRT6B |
| DSG2 | KRT80 |
| DSP | PRKCZ |
| DSTN | TGFBI |
| DTL | E2F1 |
| DTL | POLD3 |
| DUS3L | BYSL |
| DUSP3 | CTTN |
| DYNLL1 | TIMM17A |
| DZIP1 | CFL2 |
| E2F1 | DSCC1 |
| E2F2 | E2F1 |
| ECHDC1 | SSBP1 |
| EEF1A1 | NAP1L1 |
| EEF1E1 | CAPRIN1 |
| EEF1E1 | HSPD1 |
| EEF1E1 | TPRKB |
| EGR1 | CDC42BPB |
| EGR1 | FOSB |
| EHF | ELF3 |
| EHF | GRHL1 |
| EHF | LAMB3 |
| EHMT2 | AZI1 |
| EHMT2 | CCNF |
| EHMT2 | TROAP |
| EIF2AK2 | OAS3 |
| EIF2S3 | EML4 |
| EIF2S3 | SNX2 |
| EIF3J | C19orf2 |
| EIF4G2 | CREB1 |
| EIF4G2 | PTPLAD1 |
| ELAVL1 | ATP6V0A2 |
| ELAVL1 | COPS7B |
| ELAVL1 | RBM12 |
| ELF1 | IRAK4 |
| ELMO3 | ARHGEF5 |
| ELMO3 | CGN |
| ELMO3 | DSC2 |
| ELMO3 | PVRL4 |
| EML4 | ZBTB6 |
| ENO1 | YAF2 |
| ENOPH1 | ADNP |
| ENOPH1 | CCT2 |
| ENOPH1 | EXOC5 |
| ENPP5 | EPCAM |
| EPB41L1 | PLEKHA6 |
| EPHA2 | ABCC3 |
| EPHA2 | PLCD3 |
| EPHA2 | PTRF |
| EPHA2 | S100A2 |
| EPHA2 | SMURF2 |
| EPHA2 | TUFT1 |
| EPS8 | IL13RA1 |
| EPS8L2 | LAMB3 |
| EPS8L2 | MAP7 |
| ERAP1 | CASP8 |
| ERBB2 | TPD52L1 |
| ERBB3 | HOOK2 |
| ERLIN1 | DLD |
| ERLIN1 | DNAJB6 |
| ERLIN1 | IARS2 |
| ERO1L | LAMC2 |
| ESCO2 | DHX9 |
| ESCO2 | PCNA |
| ESR1 | CSH1 |
| ESR1 | CSH2 |
| EWSR1 | PASK |
| EXOC5 | RRN3 |
| EXOSC2 | CPSF6 |
| EXOSC2 | HNRNPA3 |
| EXOSC9 | DBF4 |
| EXOSC9 | NCAPG2 |
| EXOSC9 | NSL1 |
| EXOSC9 | RAD51AP1 |
| EXOSC9 | RFC4 |
| EXOSC9 | SMC2 |
| EXOSC9 | TOPBP1 |
| EXT2 | ACVR1 |
| EXT2 | RAB11FIP5 |
| EZH1 | MAPK8IP3 |
| EZH2 | POLA2 |
| EZH2 | UBE2S |
| F2RL1 | ADAM9 |
| F2RL1 | ARL14 |
| F2RL1 | C1orf106 |
| F2RL1 | CAPN1 |
| F2RL1 | DSG2 |
| F2RL1 | DSP |
| F2RL1 | ID1 |
| F2RL1 | IL18 |
| F2RL1 | LAMA5 |
| F2RL1 | LAMB3 |
| F2RL1 | PPAP2C |
| F2RL1 | TM4SF1 |
| F3 | THBS1 |
| FA2H | CLDN4 |
| FAM108B1 | ARPC5 |
| FAM108B1 | CCT2 |
| FAM108B1 | H3F3C |
| FAM108B1 | HRSP12 |
| FAM108B1 | HSPD1 |
| FAM108B1 | MED17 |
| FAM108B1 | NUDT21 |
| FAM108B1 | PIP5K1A |
| FAM108B1 | PSMD12 |
| FAM108B1 | PTPLAD1 |
| FAM108B1 | SRP9 |
| FAM108B1 | TMED2 |
| FAM108B1 | UGP2 |
| FAM108B1 | VDAC1 |
| FAM114A1 | ANXA2 |
| FAM114A1 | DSTN |
| FAM114A1 | FRMD6 |
| FAM114A1 | LGALS1 |
| FAM114A1 | RIN2 |
| FAM114A1 | RRBP1 |
| FAM114A1 | TGFB1I1 |
| FAM114A1 | TMEM184B |
| FAM54B | VKORC1 |
| FAM83F | MARVELD2 |
| FANCA | BRCA1 |
| FANCA | CDC7 |
| FANCD2 | E2F8 |
| FANCD2 | TMPO |
| FANCI | BUB1 |
| FANCI | DTL |
| FANCI | LIG1 |
| FANCI | SKP2 |
| FANCI | TOPBP1 |
| FARSA | ANAPC5 |
| FASTK | CLPTM1 |
| FASTKD2 | CNOT8 |
| FASTKD2 | MAPRE1 |
| FASTKD2 | SPTLC1 |
| FAU | GLTSCR2 |
| FBRS | RARA |
| FBXO28 | CAPN7 |
| FBXO3 | OCRL |
| FBXO31 | ZNF574 |
| FBXO5 | BIRC5 |
| FBXO5 | CENPO |
| FBXO5 | KIF2A |
| FBXO5 | TUBA1A |
| FBXW7 | MSL2 |
| FCER2 | MLL2 |
| FCER2 | PILRA |
| FCER2 | PTPRC |
| FCHO2 | ADAM9 |
| FEN1 | HELLS |
| FEN1 | KIF11 |
| FEN1 | KIF14 |
| FEN1 | RECQL4 |
| FER | PIK3CA |
| FERMT1 | GJB3 |
| FEZ2 | IGFBP6 |
| FEZ2 | LMNA |
| FEZ2 | PLIN3 |
| FEZF2 | CD163 |
| FEZF2 | TAS2R9 |
| FGD1 | NGFRAP1 |
| FGF8 | NMUR1 |
| FGFBP1 | EVPL |
| FGFBP1 | KRT15 |
| FGFBP1 | LAMA5 |
| FGFBP1 | PRRG2 |
| FGFBP1 | SCNN1A |
| FGFBP1 | SEMA4B |
| FGFBP1 | ZNF165 |
| FGFR1 | FERMT2 |
| FHL5 | GHRHR |
| FHL5 | OPRK1 |
| FHL5 | SLC13A1 |
| FKBP14 | ANXA5 |
| FKBP8 | ARL6IP4 |
| FKBP8 | CABP1 |
| FLI1 | HCLS1 |
| FLJ10038 | NSUN6 |
| FLJ44054 | ZAN |
| FLNA | CD44 |
| FLNA | IL6 |
| FLNB | PTPRF |
| FNTA | ARPC5 |
| FNTA | HNRNPC |
| FNTA | TMED2 |
| FOS | S100A10 |
| FOXA1 | CGN |
| FOXL1 | GH1 |
| FOXM1 | UBE2C |
| FOXO3 | ACP1 |
| FOXO3 | ARPC5 |
| FOXO3 | HRSP12 |
| FOXO3 | POLR2K |
| FOXO3 | PTPLAD1 |
| FOXO3 | SCYL2 |
| FRAT2 | RREB1 |
| FSTL3 | AGRN |
| FSTL3 | OSMR |
| FUS | HNRNPC |
| FUT1 | SFN |
| FUT3 | OVOL1 |
| FXYD3 | CGN |
| FZD3 | CCT2 |
| FZD3 | HSPD1 |
| FZR1 | DCTN1 |
| G3BP2 | API5 |
| G3BP2 | B3GNT1 |
| GABPA | SENP7 |
| GABPA | SRP9 |
| GART | BRIX1 |
| GART | PSMC3 |
| GART | WDR74 |
| GAS2L1 | CDC42BPB |
| GAS2L1 | TNFRSF1A |
| GBF1 | FKBP8 |
| GBF1 | MED16 |
| GBP3 | ANXA2 |
| GBP3 | LIMA1 |
| GCDH | DDX51 |
| GCFC1 | NASP |
| GDE1 | ATP6AP2 |
| GDE1 | DDX3X |
| GDE1 | MBTPS2 |
| GDE1 | STXBP3 |
| GDE1 | TSN |
| GDE1 | TTC35 |
| GDE1 | UGP2 |
| GDF15 | LTBR |
| GDI2 | API5 |
| GDI2 | CNIH |
| GDI2 | MGAT2 |
| GEMIN4 | SLC19A1 |
| GGA1 | USP20 |
| GGA1 | XAB2 |
| GGA3 | SRRM1 |
| GH1 | APBB1IP |
| GIGYF2 | MSL2 |
| GIN1 | DLAT |
| GIN1 | HSPA8 |
| GIN1 | RAB1A |
| GIN1 | TFB2M |
| GIN1 | YWHAZ |
| GINS2 | NASP |
| GIPC1 | KRT80 |
| GIPC1 | LAMA5 |
| GJB2 | EHF |
| GJB2 | F11R |
| GJB2 | ITGB6 |
| GJB3 | SEMA4B |
| GLB1 | CD63 |
| GLE1 | CD46 |
| GLE1 | GLUD2 |
| GLE1 | TBL1XR1 |
| GLE1 | TMED2 |
| GLIPR1 | COL6A2 |
| GLRX3 | HRSP12 |
| GLRX3 | HSPA8 |
| GLRX3 | SSBP1 |
| GLS2 | FUT1 |
| GLUD1 | DNAJB6 |
| GLUD1 | SDHD |
| GLUD1 | TMEM126B |
| GMNN | CCNB1 |
| GNAT1 | CD7 |
| GNAT1 | NRIP2 |
| GNG12 | BMP1 |
| GNG12 | S100A13 |
| GNG12 | S100A3 |
| GNS | SLC38A6 |
| GPR126 | ARHGEF5 |
| GPR126 | ITGA2 |
| GPR18 | AIF1 |
| GPR183 | MNDA |
| GPR3 | TACR1 |
| GPR68 | PRB3 |
| GPRC5A | DSG2 |
| GPX8 | AXL |
| GPX8 | CAPN2 |
| GPX8 | CAV2 |
| GPX8 | FBXO17 |
| GPX8 | LEPRE1 |
| GPX8 | MMP14 |
| GPX8 | PPP2R3A |
| GPX8 | PTRF |
| GPX8 | RIN2 |
| GPX8 | RND3 |
| GPX8 | S100A2 |
| GPX8 | SMURF2 |
| GPX8 | TGM2 |
| GRAP2 | THPO |
| GRB7 | ABHD11 |
| GRB7 | ALS2CL |
| GRB7 | GJB3 |
| GRHL3 | OVOL1 |
| GRHL3 | SSH3 |
| GRIK1 | THPO |
| GRTP1 | CGN |
| GRTP1 | EFNA1 |
| GRTP1 | GRHL2 |
| GRWD1 | RBM14 |
| GSN | LMNA |
| GSN | PTRF |
| GTF2A2 | ILF2 |
| GTF2A2 | PSMD10 |
| GTF2B | FNTA |
| GTF2B | NUPL2 |
| GTF2H1 | GLUD2 |
| GTF3C4 | CD46 |
| GTF3C4 | GTF3C3 |
| GTF3C4 | PNO1 |
| GTF3C4 | RPE |
| GTF3C4 | SUMO1 |
| GTSE1 | MYBL2 |
| GTSE1 | RACGAP1 |
| GYPB | OPRK1 |
| GYPB | RHAG |
| GYPE | KRT76 |
| GYPE | TAS2R8 |
| H2AFX | CCNF |
| H2AFX | POLE |
| H2AFX | TIMELESS |
| H2AFZ | RAD51AP1 |
| H2AFZ | UBE2T |
| HADH | MCM2 |
| HAUS1 | ENY2 |
| HAUS1 | RBMX |
| HBS1L | SRP9 |
| HDAC2 | KLHL23 |
| HDAC2 | MATR3 |
| HELLS | PCNA |
| HELLS | RFC2 |
| HELLS | SFPQ |
| HELLS | TOPBP1 |
| HELLS | ZNF107 |
| HEXB | ADAM9 |
| HFE | IL17RC |
| HIP1R | PTCRA |
| HLA-G | CD58 |
| HMGB1 | CDC7 |
| HMGB1 | E2F8 |
| HMGB1 | HNRNPA2B1 |
| HMGB1 | POLA2 |
| HMGB1 | RBBP4 |
| HMGB1 | USP1 |
| HMGB2 | BCLAF1 |
| HMGB2 | FUS |
| HMGB2 | HNRNPA1 |
| HMGB2 | HNRNPA3 |
| HMGB2 | SKP2 |
| HMGB2 | TIMELESS |
| HMGB2 | USP37 |
| HMMR | PCNA |
| HNRNPA1 | CDC7 |
| HNRNPA2B1 | DLGAP5 |
| HNRNPA2B1 | HMGB2 |
| HNRNPA2B1 | YBX1 |
| HNRNPC | DDX46 |
| HNRNPC | SKIV2L2 |
| HNRNPC | TBL1XR1 |
| HNRNPC | TPR |
| HNRNPC | YBX1 |
| HNRNPD | DDX46 |
| HNRNPD | HNRNPA1 |
| HNRNPD | NRF1 |
| HNRNPD | NUP160 |
| HNRNPD | TOP2A |
| HNRNPD | TOPBP1 |
| HNRNPF | CANX |
| HNRNPF | CLINT1 |
| HNRNPF | CREB1 |
| HNRNPF | DLG1 |
| HNRNPF | HNRNPA2B1 |
| HNRNPF | MOCS3 |
| HNRNPF | SLC25A40 |
| HNRNPF | TMEM48 |
| HNRNPF | UCHL5 |
| HNRNPH3 | FUS |
| HNRNPH3 | HNRNPM |
| HNRNPM | C2orf44 |
| HNRNPR | CTCF |
| HNRNPR | RBM14 |
| HOXB8 | GRIA3 |
| HOXD12 | GRM4 |
| HRSP12 | TFB2M |
| HRSP12 | UBXN4 |
| HSD3B2 | POU4F3 |
| HSD3B2 | THPO |
| HSF2 | C2orf44 |
| HSP90AB1 | PTPLAD1 |
| HSPA4 | HSPA8 |
| HTN1 | TAS2R8 |
| HTRA1 | IGFBP3 |
| HTRA1 | KIRREL |
| HVCN1 | APBB1IP |
| IARS | YARS |
| IARS2 | MTFR1 |
| IARS2 | PEX2 |
| IARS2 | RNF14 |
| IARS2 | TAF12 |
| IBSP | KLK2 |
| ICAM1 | HLA-C |
| IDI1 | INSIG1 |
| IFFO1 | MAP3K3 |
| IFIT1 | IFI27 |
| IFNGR1 | MMADHC |
| IFNGR1 | SLC38A2 |
| IGFBP7 | CD109 |
| IGFBP7 | ITGA5 |
| IKBIP | CALU |
| IKBIP | TPST1 |
| IKBIP | WBP5 |
| IL16 | CD84 |
| IL16 | LILRA2 |
| IL17RB | EPCAM |
| IL17RB | GLS2 |
| IL18 | SERPINB5 |
| IL18 | SLC16A5 |
| IL20RA | STX19 |
| IL3RA | AIF1 |
| ILF3 | ARHGAP19 |
| ILF3 | CTCF |
| ILF3 | HNRNPH1 |
| ILKAP | SF1 |
| IMPAD1 | ITFG1 |
| IMPAD1 | RAB1A |
| IMPAD1 | UBE2H |
| INADL | GPR56 |
| INTS12 | CCDC76 |
| INTS12 | HBS1L |
| INTS12 | POLR2K |
| INTS12 | UCHL5 |
| IRF2BP1 | ZNF335 |
| IRF6 | GRHL3 |
| IRF7 | IRF9 |
| ISG15 | OASL |
| ITGA2 | ADAM9 |
| ITGA2 | BCAR3 |
| ITGA2 | SLC2A1 |
| ITGA3 | SDC4 |
| ITGAV | AHNAK |
| ITGB3BP | MSH2 |
| ITSN1 | FERMT2 |
| JPH3 | POLH |
| KCND3 | EMX1 |
| KCND3 | MEOX2 |
| KCND3 | OMD |
| KCND3 | PPP1R3A |
| KCNJ5 | ALDOB |
| KCNJ5 | ZNF335 |
| KCTD11 | ADAM9 |
| KCTD13 | IRF2BP1 |
| KCTD13 | SMARCC2 |
| KDELR2 | THBS1 |
| KDELR3 | ARL1 |
| KDELR3 | GPRC5A |
| KDELR3 | HEBP1 |
| KDM3A | MATR3 |
| KDM4B | POGZ |
| KDM4B | SMARCC2 |
| KDM6B | POLR1B |
| KDM6B | RHOT2 |
| KERA | SLC13A1 |
| KHSRP | CPSF1 |
| KIAA0101 | MCM3 |
| KIAA0101 | PCNA |
| KIAA0101 | POLE2 |
| KIAA0101 | PPIH |
| KIAA0101 | SMC4 |
| KIAA0101 | SNRPA |
| KIAA0101 | SNRPD1 |
| KIAA0284 | GIPC1 |
| KIAA0664 | SLC25A10 |
| KIAA0664 | SOLH |
| KIAA0664 | TRAP1 |
| KIAA0664 | USP36 |
| KIAA0913 | PHF1 |
| KIAA1033 | DNAJC10 |
| KIAA1279 | RAB1A |
| KIAA1522 | GOLT1A |
| KIAA1522 | NR2F6 |
| KIAA1522 | TSKU |
| KIAA1609 | BCL9L |
| KIAA1609 | TJP1 |
| KIAA1731 | PPIG |
| KIF11 | GINS1 |
| KIF11 | PCNA |
| KIF11 | POLE2 |
| KIF11 | RFC4 |
| KIF11 | SMC2 |
| KIF11 | TYMS |
| KIF15 | BRCA1 |
| KIF15 | CKS1B |
| KIF15 | CPSF6 |
| KIF15 | WDR67 |
| KIF18A | CENPA |
| KIF18A | MSH2 |
| KIF18A | ZWILCH |
| KIF20A | UBE2S |
| KIF20B | E2F1 |
| KIF20B | UBE2C |
| KIF23 | GINS1 |
| KIF23 | PLK1 |
| KIF2C | AURKA |
| KIF2C | CKS1B |
| KIF2C | MYBL2 |
| KIF2C | PLK1 |
| KIF2C | RAD51AP1 |
| KIF2C | SMC2 |
| KIF2C | TIMELESS |
| KIFC1 | CIT |
| KIFC1 | NCAPD2 |
| KLF4 | CD9 |
| KLF4 | GPRC5A |
| KLF5 | DDR1 |
| KLF5 | EDN1 |
| KLF5 | FOS |
| KLF5 | GPRC5A |
| KLF5 | MET |
| KLF5 | PLEK2 |
| KLF5 | PRRG2 |
| KLHL8 | LIN9 |
| KLHL8 | MSL2 |
| KLHL8 | ZNF678 |
| KNTC1 | CDCA7 |
| KNTC1 | CENPA |
| KNTC1 | LIG1 |
| KNTC1 | NUP153 |
| KRI1 | CPSF6 |
| KRI1 | DDX55 |
| KRI1 | NOP56 |
| KRI1 | POGZ |
| KRI1 | RBM14 |
| KRT16 | GJB3 |
| KRT19 | BSPRY |
| LAMA4 | GPX8 |
| LAMB2 | CDC42BPB |
| LAMB2 | PTPN21 |
| LAMB2 | RAB11FIP5 |
| LAMB2 | RRBP1 |
| LAMB4 | TRAT1 |
| LAPTM4A | CETN2 |
| LAPTM4A | PRSS23 |
| LARP1 | IPO4 |
| LARP6 | NMT2 |
| LARP7 | ACP1 |
| LARP7 | GLUD2 |
| LARP7 | HRSP12 |
| LARP7 | RANBP2 |
| LARP7 | UGP2 |
| LATS2 | DAB2 |
| LBR | TCERG1 |
| LCORL | NAA38 |
| LDB1 | PBX2 |
| LEPROT | ANXA1 |
| LEPROT | PRSS23 |
| LGALS1 | FOSL1 |
| LHFP | JAZF1 |
| LIF | EHD2 |
| LIG1 | BIRC5 |
| LIG1 | NAP1L4 |
| LIG1 | RBM14 |
| LIN7C | RAB1A |
| LIN9 | ATAD5 |
| LMAN1 | EXOC5 |
| LMAN1 | HSPD1 |
| LMAN1 | PIK3CA |
| LMAN1 | PIP5K1A |
| LMAN1 | RPE |
| LMAN1 | YAF2 |
| LMBRD1 | ARPC5 |
| LMBRD1 | CD46 |
| LMBRD1 | HRSP12 |
| LMNB1 | CENPA |
| LMNB1 | MYBL2 |
| LMNB2 | POLE |
| LMNB2 | RBM14 |
| LNPEP | MBNL1 |
| LOC81691 | KIF15 |
| LOX | ADAMTS1 |
| LOXL2 | DAB2 |
| LOXL2 | NCS1 |
| LOXL2 | RAI14 |
| LOXL2 | RND3 |
| LPAL2 | HOXB8 |
| LRCH4 | GGA3 |
| LRRC1 | HOOK2 |
| LRRC40 | RAD51AP1 |
| LRRC8E | GPRC5A |
| LRTM1 | PKD2L2 |
| LSM7 | NASP |
| LSM7 | POLE |
| LSM7 | RBM14 |
| LSM7 | SKP2 |
| LSP1 | IKZF1 |
| LTBR | CSTB |
| LTBR | GALE |
| LTBR | PLEK2 |
| LUC7L3 | PRPF4B |
| LUC7L3 | RBMX |
| LY6G6D | FETUB |
| LYAR | RIOK1 |
| MAD2L1 | HNRNPA1 |
| MAD2L1 | MCM2 |
| MAD2L1 | UBE2T |
| MADD | FAM193B |
| MAGEL2 | OR10J1 |
| MAGOH | HNRNPC |
| MAK16 | POLR1B |
| MANEA | CREB1 |
| MANEA | HNRNPA2B1 |
| MAP1LC3B | HSPA13 |
| MAP1S | MAP3K11 |
| MAP1S | NCOR2 |
| MAP1S | NOC4L |
| MAP1S | SMARCC2 |
| MAP2K4 | SENP2 |
| MAP2K7 | GGA3 |
| MAP2K7 | POGZ |
| MAP2K7 | SLC22A8 |
| MAP2K7 | TACR1 |
| MAP3K3 | ZBTB17 |
| MAP3K7 | DNAJB6 |
| MAP3K7 | HSPA4 |
| MAP3K7 | POLR3C |
| MAP3K7 | SLC30A5 |
| MAP3K7 | YAF2 |
| MAP3K9 | CLDN4 |
| MAP7 | ARHGEF5 |
| MAP7 | CGN |
| MAP7 | EFNA1 |
| MAP7 | EXPH5 |
| MAP7 | GRHL2 |
| MAP7 | PVRL4 |
| MAPK1 | ATP6V1A |
| MAPK8IP3 | C11orf2 |
| MARCH5 | ILF2 |
| MARCH5 | RHOA |
| MARCH5 | SSBP1 |
| MARCH7 | DLD |
| MARCH7 | SRP9 |
| MARS2 | COX5A |
| MARVELD3 | CHDH |
| MARVELD3 | GRHL3 |
| MARVELD3 | PVRL4 |
| MBD3 | DCTN1 |
| MBD3 | MED24 |
| MBTPS1 | CALU |
| MBTPS2 | PSMD10 |
| MCM10 | ARHGAP11A |
| MCM10 | CCNB2 |
| MCM10 | CTCF |
| MCM10 | FANCG |
| MCM10 | RAD51 |
| MCM10 | SMC2 |
| MCM3 | HNRNPR |
| MCM3 | LMNB1 |
| MCM3 | NUDT21 |
| MCM3 | RFC4 |
| MCM3 | USP39 |
| MCM5 | MYBL2 |
| MCM5 | RFC2 |
| MDC1 | CPSF6 |
| MDC1 | TROAP |
| MDM4 | TCERG1 |
| ME2 | EXOC5 |
| ME2 | NONO |
| MED16 | DCTN1 |
| MED21 | HRSP12 |
| MED4 | SRP9 |
| MED7 | HRSP12 |
| MFNG | PTPN6 |
| MGC16275 | POLR1B |
| MGRN1 | HCFC1 |
| MGST2 | F11R |
| MIER2 | DCTN1 |
| MKI67 | PCNA |
| MLF1IP | CDC7 |
| MLF1IP | MCM2 |
| MLF1IP | RRM2 |
| MLF1IP | SKP2 |
| MLLT10 | SF3B1 |
| MLLT10 | ZNF273 |
| MMADHC | TMEM126B |
| MND1 | ANP32E |
| MND1 | ATAD5 |
| MND1 | CDC7 |
| MND1 | NCAPH |
| MND1 | TOPBP1 |
| MPDZ | SDC2 |
| MPZL2 | LAD1 |
| MPZL2 | RNF39 |
| MPZL2 | ST6GALNAC1 |
| MRFAP1L1 | ILF2 |
| MRFAP1L1 | TBL1XR1 |
| MRPL12 | WDR77 |
| MRPL13 | ARFGEF2 |
| MRPL13 | GLUD2 |
| MRPL13 | PRKAA1 |
| MRPL18 | ENOPH1 |
| MRPL18 | MRPL3 |
| MRPL18 | TPRKB |
| MRPL2 | USP36 |
| MRPL3 | GART |
| MRPL3 | NUS1 |
| MRPL37 | MRPL38 |
| MRPL39 | EXOC5 |
| MRPL39 | NMD3 |
| MRPL42 | HNRNPA2B1 |
| MRPL42 | YBX1 |
| MRPS15 | DNAJA2 |
| MRPS2 | PHB2 |
| MRPS25 | ING5 |
| MRPS28 | PNO1 |
| MRPS7 | MRPS12 |
| MT2A | PLAT |
| MT2A | PRKCDBP |
| MT2A | S100A3 |
| MT4 | PLAUR |
| MTA1 | BAZ1B |
| MTF2 | DBF4 |
| MTF2 | MLF1IP |
| MTF2 | TOPBP1 |
| MTF2 | ZNF678 |
| MVP | PDXK |
| MYB | ATAD5 |
| MYB | DEPDC5 |
| MYB | MARS2 |
| MYB | RMND5A |
| MYB | SEMA4D |
| MYB | SIDT1 |
| MYH13 | CCL24 |
| MYH13 | HAMP |
| MYH14 | PTPRU |
| MYH14 | SSH3 |
| MYH2 | ACSM1 |
| MYH3 | ADCY8 |
| MYH4 | FETUB |
| MYH4 | KCNJ9 |
| MYH4 | MLL2 |
| MYH7 | CD79B |
| MYH9 | PTPN14 |
| MYL12A | CAV2 |
| MYL12A | FZD6 |
| MYL12A | S100A11 |
| MYO1C | EHD2 |
| MYO1C | PDXK |
| MYO1C | PLEC |
| MYO1C | TEAD3 |
| MYO5C | LRRC1 |
| MYO6 | CGN |
| MYOF | ADAM9 |
| MYOF | AGRN |
| MYOF | AXL |
| MYOF | CLIP1 |
| MYOF | CSTB |
| MYOF | CYR61 |
| MYOF | MYO1E |
| MYOF | PINK1 |
| MYOF | PPP2R3A |
| MYOF | TNFAIP2 |
| MYOF | TRIP6 |
| NAA15 | CCNC |
| NAA15 | CCT2 |
| NAA15 | EEF1E1 |
| NAA16 | RSBN1 |
| NAA16 | ZNF138 |
| NAA50 | CD46 |
| NAA50 | GDE1 |
| NAE1 | CCDC138 |
| NAP1L4 | HNRNPUL1 |
| NAP1L4 | PABPN1 |
| NARS2 | ZBTB6 |
| NARS2 | ZNF227 |
| NASP | HNRNPA1 |
| NASP | TIMELESS |
| NAT10 | RPIA |
| NBEAL2 | ADRBK1 |
| NBEAL2 | MLLT6 |
| NBEAL2 | PPP2R5A |
| NCAPD2 | RAD51 |
| NCAPD3 | CCNF |
| NCAPD3 | UBE2C |
| NCAPG | CDCA3 |
| NCAPG | CENPF |
| NCAPG | CENPI |
| NCAPG | CKAP5 |
| NCAPG | CKS1B |
| NCAPG | HNRNPA2B1 |
| NCAPG | MYBL2 |
| NCAPG | NCAPG2 |
| NCAPG | NCAPH |
| NCAPG | POLE2 |
| NCAPG | RFC2 |
| NCAPG | ZWINT |
| NCAPH2 | MYBL2 |
| NCAPH2 | ZNF335 |
| NCBP1 | RBM14 |
| NCBP1 | TMED2 |
| NCBP2 | C20orf30 |
| NCF4 | LAIR1 |
| NCLN | DCTN1 |
| NCR1 | FETUB |
| NCR1 | PRKACG |
| NDC80 | BARD1 |
| NDC80 | CENPF |
| NDC80 | PCNA |
| NDC80 | POLE2 |
| NDC80 | RFC5 |
| NDUFAF4 | DLAT |
| NDUFAF4 | DLD |
| NDUFAF4 | LYRM7 |
| NDUFAF4 | MATR3 |
| NDUFAF4 | MRPL3 |
| NDUFAF4 | SKIV2L2 |
| NDUFAF4 | TPRKB |
| NDUFS4 | HSPA8 |
| NEIL3 | POLE2 |
| NEIL3 | RAD51AP1 |
| NEIL3 | RRM2 |
| NEIL3 | SMC4 |
| NEK2 | TPX2 |
| NEUROG2 | MNDA |
| NEUROG2 | PPP1R3A |
| NEXN | FBN1 |
| NFATC3 | MCM2 |
| NFYB | CCNT2 |
| NLE1 | WDR77 |
| NOC2L | RHOT2 |
| NOC2L | SMG5 |
| NOC3L | PAK1IP1 |
| NOL11 | CCDC58 |
| NOL11 | CHEK1 |
| NOL11 | RG9MTD1 |
| NOL11 | ZNF670 |
| NOL12 | LIG1 |
| NOL12 | PPAN |
| NOL12 | SMARCC2 |
| NOLC1 | PHB2 |
| NONO | PHF6 |
| NOP56 | CIRH1A |
| NOP56 | FUS |
| NOP56 | NCL |
| NPM3 | PHB2 |
| NPNT | EPCAM |
| NPTN | LPP |
| NRF1 | CKAP5 |
| NSMCE4A | MCM2 |
| NSMCE4A | STRBP |
| NT5E | CD59 |
| NT5E | RAI14 |
| NT5E | S100A3 |
| NTN4 | CFB |
| NUDT21 | ARFGEF1 |
| NUDT21 | FH |
| NUDT21 | GMPR2 |
| NUDT21 | SCYL2 |
| NUDT21 | SLC25A40 |
| NUDT21 | TMED2 |
| NUDT21 | UBC |
| NUDT21 | ZNF227 |
| NUDT21 | ZNF780A |
| NUP160 | MSH2 |
| NUP160 | ZNF670 |
| NUP188 | FUS |
| NUP54 | CNBP |
| NUP54 | HAT1 |
| NUSAP1 | CENPI |
| NUSAP1 | DSCC1 |
| NUSAP1 | NCAPH |
| OMD | IMPG1 |
| OPTN | IFI35 |
| OR1I1 | SLC4A1 |
| ORM1 | KCNH6 |
| OSGEPL1 | RAD17 |
| OSGEPL1 | SKIV2L2 |
| OSTM1 | GNS |
| OSTM1 | HEXB |
| OVOL2 | CLDN3 |
| P4HA2 | COL4A2 |
| P4HA2 | S100A13 |
| P4HA2 | ULBP2 |
| PABPC3 | AZIN1 |
| PACS2 | STRN4 |
| PAICS | HNRNPC |
| PAICS | MCM2 |
| PALLD | TGFB1I1 |
| PAPOLG | MATR3 |
| PAPOLG | UBR5 |
| PAPOLG | ZCCHC11 |
| PARP1 | ATAD5 |
| PARVA | PLOD1 |
| PARVA | SMURF2 |
| PARVG | CD79A |
| PATZ1 | AKAP8L |
| PATZ1 | DDX51 |
| PATZ1 | POLE |
| PBK | DLGAP5 |
| PBK | ECT2 |
| PBK | POLE2 |
| PBK | RFC4 |
| PBK | TYMS |
| PBOV1 | PILRA |
| PCNA | CENPA |
| PCNA | DHX9 |
| PCNA | KIF11 |
| PCNA | ZWINT |
| PCNT | FANCA |
| PDCD11 | SLC19A1 |
| PDE4C | SFTPB |
| PDE6C | KCND3 |
| PDGFC | ITGAV |
| PDGFC | PLOD2 |
| PDGFC | SNX21 |
| PDIK1L | CTCF |
| PDIK1L | ZNF124 |
| PDSS1 | RAD51 |
| PDSS1 | SRRT |
| PDX1 | CRP |
| PDX1 | GRM4 |
| PDX1 | PHKG1 |
| PDX1 | PPP1R3A |
| PERP | ATP8B1 |
| PERP | SH2D3A |
| PES1 | CARM1 |
| PES1 | RRP1 |
| PES1 | SNAPC4 |
| PES1 | TRMT1 |
| PEX2 | DNAJB6 |
| PEX2 | PNO1 |
| PFKFB2 | INADL |
| PFN1 | ACTB |
| PGAM5 | CHAC2 |
| PGAM5 | TBRG4 |
| PGGT1B | CREB1 |
| PGGT1B | DNM1L |
| PHB2 | SNRPA |
| PHF11 | CTSS |
| PHF15 | TAPBP |
| PHF2 | KDM3B |
| PHF7 | TACR1 |
| PHLDB1 | PTPN14 |
| PHOX2B | SLC13A1 |
| PIAS4 | DCTN1 |
| PIAS4 | POLR1B |
| PICALM | PSEN1 |
| PIK3C2A | MAP4K3 |
| PIK3C2A | VAMP3 |
| PIK3CA | ACP1 |
| PIK3CA | ARPC5 |
| PIK3CA | PRPF18 |
| PILRA | MYH6 |
| PIN1 | TUBB |
| PINK1 | PTRF |
| PKMYT1 | C11orf2 |
| PKMYT1 | SIVA1 |
| PKMYT1 | STIL |
| PKP3 | GJB3 |
| PKP3 | LAMC2 |
| PKP3 | MAPK13 |
| PKP3 | PRSS16 |
| PLA2G1B | BMP8A |
| PLAT | CD63 |
| PLCG2 | TMC8 |
| PLEKHG3 | LAMA5 |
| PLEKHG3 | PTPRU |
| PLEKHG3 | TSPAN1 |
| PLK1 | RAD54L |
| PLK2 | ADAM9 |
| PLK2 | EPHA2 |
| PLK2 | RIN2 |
| PLK2 | S100A2 |
| PLK2 | SSH3 |
| PLK4 | CENPN |
| PLK4 | CKS1B |
| PLK4 | LBR |
| PLK4 | MCM2 |
| PLK4 | NCAPG2 |
| PLK4 | RIF1 |
| PLK4 | SKP2 |
| PLK4 | UBE2T |
| PLLP | MPZL2 |
| PLOD1 | CALU |
| PLOD1 | CD63 |
| PLOD1 | RRAS |
| PLOD3 | TMEM43 |
| PLXNB2 | CTNND1 |
| PLXNB2 | TSKU |
| PM20D2 | YEATS4 |
| PNLIPRP1 | LILRB3 |
| POLA2 | CEP152 |
| POLA2 | EXO1 |
| POLA2 | KIAA0101 |
| POLA2 | KIF14 |
| POLD1 | RBM14 |
| POLE | FANCC |
| POLE | SNRNP70 |
| POLE2 | BRCA1 |
| POLE2 | CDC25C |
| POLE2 | MCM6 |
| POLE2 | RFC2 |
| POLH | CRY2 |
| POLH | THPO |
| POLH | TMEM19 |
| POLR1B | POLH |
| POLR2A | ZNF574 |
| POLR2L | AP2S1 |
| POLR3F | ASAP1 |
| POLR3K | HNRNPA2B1 |
| POT1 | CNBP |
| POT1 | RAB23 |
| POT1 | RAD17 |
| POU2F1 | CHD4 |
| PPAN | DDX51 |
| PPIC | C6orf145 |
| PPIC | CAPN2 |
| PPIC | EDN1 |
| PPIC | S100A13 |
| PPIC | SERPINH1 |
| PPL | C1orf172 |
| PPL | TINAGL1 |
| PPP1CC | CCDC138 |
| PPP1CC | CPNE1 |
| PPP1R13L | ATP8B1 |
| PPP1R15A | CEBPB |
| PPP1R3A | MYH6 |
| PPP1R8 | NXF1 |
| PPP2R3C | MATR3 |
| PPP5C | FUS |
| PPRC1 | PIAS4 |
| PPRC1 | SNAPC4 |
| PRC1 | DTL |
| PRIM1 | STIL |
| PRKCDBP | S100A6 |
| PRKDC | HAT1 |
| PRKDC | HSPA4 |
| PRKDC | HSPD1 |
| PRKDC | MSH6 |
| PRKRA | HRSP12 |
| PRKRA | SENP2 |
| PRM2 | CATSPERG |
| PRNP | BACH1 |
| PRNP | KIRREL |
| PRNP | PHLDA1 |
| PRNP | S100A2 |
| PRNP | TMED2 |
| PRNP | UBC |
| PRNP | ULBP2 |
| PROL1 | ALDOB |
| PRPF38A | CTCF |
| PRPF38A | RBM14 |
| PRR14 | BRF1 |
| PRR14 | UBTF |
| PRR5 | DDR1 |
| PRR5 | KRT6B |
| PRR5 | MAPK13 |
| PRR5 | PTPRF |
| PRRG2 | CXCL16 |
| PRRG2 | SSH3 |
| PSAP | CTSB |
| PSEN1 | ADAM10 |
| PSEN1 | RNF14 |
| PSEN1 | SYPL1 |
| PSMA1 | ARPC5 |
| PSMA1 | CREB1 |
| PSMA1 | HRSP12 |
| PSMA1 | IARS2 |
| PSMA1 | MED17 |
| PSMA1 | POLR2K |
| PSMA1 | PPP1R2 |
| PSMA1 | PSMA2 |
| PSMA1 | PTPLAD1 |
| PSMA1 | RARS |
| PSMA1 | RPF1 |
| PSMA1 | UGP2 |
| PSMA5 | ARPC5 |
| PSMC3 | CD46 |
| PSMC3 | PTK2 |
| PSMC3 | RAB1A |
| PSMC3 | TSN |
| PSMC3 | YAF2 |
| PSMC3 | YWHAZ |
| PSMD12 | HRSP12 |
| PSMD12 | VAMP3 |
| PSMD6 | DNAJA2 |
| PSMD6 | FH |
| PSMD6 | MRPL3 |
| PSMD6 | TPRKB |
| PSMD6 | UGP2 |
| PSMD6 | ZNF227 |
| PSRC1 | CCNF |
| PTBP1 | CPSF6 |
| PTBP1 | NASP |
| PTBP1 | RBM14 |
| PTGES3 | RRM1 |
| PTP4A1 | PSEN1 |
| PTPLA | PTRF |
| PTPLAD1 | DLAT |
| PTPLAD1 | GLUD2 |
| PTPLAD1 | RPAP3 |
| PTPN12 | PLAUR |
| PTPN22 | TRAF3IP3 |
| PTPN3 | C1orf172 |
| PTPN6 | AP1G2 |
| PTPRF | SEMA4B |
| PTRF | CFL2 |
| PTRF | DRAP1 |
| PTRF | HMGA2 |
| PTRF | LAMC1 |
| PTRF | MMP14 |
| PUM2 | ZBTB6 |
| PURG | OR10J1 |
| PXN | MET |
| RAB1A | ASAP1 |
| RAB1A | COPS5 |
| RAB1A | DCTN6 |
| RAB1A | GDE1 |
| RAB1A | IFNGR1 |
| RAB1A | IMPAD1 |
| RAB1A | MTFR1 |
| RAB1A | PEX2 |
| RAB1A | PICALM |
| RAB1A | PTK2 |
| RAB1A | RIOK3 |
| RAB1A | TMED10 |
| RAB28 | HNRNPC |
| RAB28 | PIK3CA |
| RAB28 | PSMC3 |
| RAB5A | ARFGEF2 |
| RAB5A | CLIP1 |
| RAB5A | CNIH |
| RAB5A | DNAJB6 |
| RAB5A | PDCD10 |
| RAC1 | CTTN |
| RAC2 | RUNX1 |
| RAD17 | C19orf2 |
| RAD17 | CLDND1 |
| RAD17 | DLD |
| RAD17 | MAPRE1 |
| RAD17 | PIK3CA |
| RAD17 | RBM12 |
| RAD17 | SSBP1 |
| RAD17 | ZNF780A |
| RAD23B | CD46 |
| RAD23B | HRSP12 |
| RAD23B | ITCH |
| RAD23B | PNO1 |
| RAD51 | LIG1 |
| RAD51 | TOP2A |
| RAD51AP1 | LMNB1 |
| RAD54L | HMGB1 |
| RAD54L | RAD51AP1 |
| RAD54L | XRCC3 |
| RALB | LMNA |
| RALGPS1 | OVOL2 |
| RANBP1 | GART |
| RANBP3 | BAZ1B |
| RANBP3 | DCTN1 |
| RANBP3 | SMARCC2 |
| RANBP9 | B3GNT2 |
| RANBP9 | CCNG2 |
| RARS | RAB23 |
| RASSF8 | NNMT |
| RBM10 | EDC4 |
| RBM10 | FAM193B |
| RBM10 | MED12 |
| RBM10 | MXD3 |
| RBM10 | SMG5 |
| RBM10 | SUZ12 |
| RBM10 | UBE2O |
| RBM10 | USP22 |
| RBM12 | SRP9 |
| RBM15 | DDX11 |
| RBM15 | LBR |
| RBM26 | CCAR1 |
| RBM26 | HNRNPA3 |
| RBM26 | ZRANB2 |
| RBM47 | PLS1 |
| RBPMS | LPP |
| RBPMS | RHOC |
| RC3H2 | SRP9 |
| RCC2 | CTCF |
| RCOR3 | PHF21A |
| RDH13 | ESRRA |
| REXO4 | PUS1 |
| RFC3 | HNRNPA2B1 |
| RFC3 | ILF2 |
| RFC3 | MCM2 |
| RFC3 | MSH2 |
| RFC3 | NASP |
| RFC3 | PSMC3 |
| RFC3 | SLC25A19 |
| RFC3 | USP39 |
| RFC3 | YBX1 |
| RFC4 | TOP2A |
| RFC5 | CHAC2 |
| RFC5 | HNRNPA2B1 |
| RFNG | ZNF768 |
| RFXAP | LBR |
| RFXAP | PRPF38A |
| RHBDF1 | LGALS3 |
| RHBDF1 | LRRC8E |
| RHBDF1 | TRIM16L |
| RHOA | ATP6V1A |
| RHOA | KLHL12 |
| RHOA | MGAT2 |
| RHOA | RPAP3 |
| RHOC | CPA4 |
| RHOC | S100A13 |
| RHOT2 | SMARCC2 |
| RIF1 | CCAR1 |
| RIPK4 | SSH3 |
| RMI1 | DHFR |
| RMI1 | MCM6 |
| RMND5A | PARP1 |
| RNASE2 | KCNH6 |
| RNASEH2A | BRCA1 |
| RNASEH2A | OIP5 |
| RNASEH2A | TUBA1A |
| RNF138 | MSL2 |
| RNF138 | NUP160 |
| RNF146 | C14orf166 |
| RNF146 | CMAS |
| RNF146 | EIF2B1 |
| RNF146 | IARS2 |
| RNF146 | ILF2 |
| RNF146 | MATR3 |
| RNF146 | MMADHC |
| RNF146 | NFYB |
| RNF146 | PSMA2 |
| RNF146 | RAD23B |
| RNF146 | SLC38A2 |
| RNF146 | UBC |
| RNF146 | YAF2 |
| RNF146 | YIPF4 |
| RNF219 | HNRNPA3 |
| RNF38 | PUM2 |
| RNF6 | SDHD |
| RPE | CCT5 |
| RPL11 | EIF2B1 |
| RPL11 | MSH2 |
| RPL11 | PRKDC |
| RPL11 | RPS14 |
| RPL27A | RPS14 |
| RPL36 | NACA |
| RPL36 | NACAP1 |
| RPL36 | RPS11 |
| RPL36 | RPS16 |
| RPL36 | RPS5 |
| RPL5 | HNRNPA1 |
| RPP14 | PNO1 |
| RPP14 | TMED2 |
| RPS24 | RPL10A |
| RPS24 | RPL11 |
| RPS24 | RPS11 |
| RPS6 | RIMS2 |
| RPS6KA1 | TMC8 |
| RPS6KB1 | ARPC5 |
| RPS6KB1 | DLD |
| RPS6KB1 | MLLT10 |
| RRAGB | ASAP1 |
| RRAS2 | MYO1E |
| RRM1 | ENO1 |
| RRM1 | LRRC40 |
| RRM1 | SRP9 |
| RRM1 | TOPBP1 |
| RRM1 | ZNF273 |
| RRM2 | CCNF |
| RRM2 | FEN1 |
| RRN3 | MAT2A |
| RRP1B | GEMIN5 |
| RRP1B | RPIA |
| RUSC2 | CAP2 |
| RYK | RNF11 |
| S100P | EVPL |
| SAFB | FUBP1 |
| SAFB | HNRNPA3 |
| SAFB | LUC7L3 |
| SAFB | MATR3 |
| SAFB | NUP160 |
| SAFB | POLE |
| SAFB | RBM12 |
| SAFB | RBM14 |
| SAFB | SFPQ |
| SAFB | SKP2 |
| SAFB2 | CNNM3 |
| SAFB2 | CPSF1 |
| SAMD1 | HNRNPR |
| SAMD1 | KHDRBS1 |
| SARS | DDIT3 |
| SART3 | KHDRBS1 |
| SBNO1 | TARDBP |
| SCAI | PDIK1L |
| SCEL | VGLL1 |
| SCN2B | THPO |
| SCNN1A | C1orf172 |
| SCNN1A | RNF39 |
| SCYL2 | DSCR3 |
| SCYL2 | HSPD1 |
| SCYL2 | PSMD6 |
| SCYL2 | SLC25A40 |
| SCYL2 | UBE2H |
| SCYL2 | ZNF780A |
| SDC1 | CDS1 |
| SDC1 | KIAA1217 |
| SDF4 | P4HB |
| SDHB | HNRNPF |
| SDHD | HSPD1 |
| SEC24B | GLUD2 |
| SEC24B | SRP9 |
| SEC24B | UBXN4 |
| SEL1L | CD164 |
| SEMA3B | GPRC5A |
| SEMA4B | GJB2 |
| SENP2 | CWF19L1 |
| SENP2 | DNAJC10 |
| SENP2 | STRAP |
| SEP15 | B3GNT1 |
| SEP15 | CLINT1 |
| SEP15 | KLHL12 |
| SEP15 | YAF2 |
| SERINC1 | MMADHC |
| SERINC1 | PTK2 |
| SERINC1 | SLC38A2 |
| SERINC1 | UBA6 |
| SERPINB5 | GPRC5A |
| SERPINB5 | RNF39 |
| SERPINB6 | EPHA2 |
| SEZ6L | FOXN4 |
| SF1 | USP7 |
| SF1 | ZC3H7B |
| SF3A2 | CAD |
| SF3A2 | GJA8 |
| SF3A2 | POLR1B |
| SF3A2 | TNK2 |
| SFI1 | C19orf40 |
| SFI1 | POLE |
| SFI1 | TMEM19 |
| SFN | LLGL2 |
| SFN | RNF43 |
| SFPQ | RBM26 |
| SFTPC | LILRB3 |
| SFXN4 | COX5A |
| SGCB | LRP12 |
| SGMS2 | RAB11FIP5 |
| SGTA | CCNT1 |
| SGTA | DCTN1 |
| SGTA | POLR1B |
| SGTA | POMT2 |
| SGTA | RBM14 |
| SGTA | SMARCC2 |
| SH2D4A | B4GALT1 |
| SH2D4A | SLPI |
| SH3BGRL3 | DRAP1 |
| SH3BGRL3 | PTRF |
| SH3D19 | ANXA4 |
| SH3D19 | S100A6 |
| SH3GL1 | PLEC |
| SH3RF1 | RND3 |
| SHB | ANXA2 |
| SHPRH | ANKRD46 |
| SHPRH | ZNF124 |
| SHROOM3 | CXCL16 |
| SIGLEC7 | DPEP2 |
| SIGLEC7 | PVRL1 |
| SIGLEC8 | SPI1 |
| SIL1 | LRP10 |
| SIX3 | KLF1 |
| SKIV2L2 | EML4 |
| SKIV2L2 | GLUD2 |
| SKIV2L2 | PNO1 |
| SKIV2L2 | TMED2 |
| SKP1 | RAB1A |
| SLBP | ARPC5 |
| SLBP | MSH6 |
| SLBP | RFC4 |
| SLBP | ZNF227 |
| SLBP | ZWINT |
| SLC12A1 | SLC13A1 |
| SLC19A1 | BRF1 |
| SLC22A13 | TAS2R9 |
| SLC25A32 | TFB2M |
| SLC2A1 | ABCC3 |
| SLC2A1 | S100A16 |
| SLC30A5 | ARPC1A |
| SLC30A5 | C20orf30 |
| SLC30A5 | CALU |
| SLC30A5 | MAP3K7 |
| SLC30A5 | RAB1A |
| SLC30A5 | TMEM126B |
| SLC35A2 | TMED9 |
| SLC35B4 | CCDC88A |
| SLC35D2 | MET |
| SLC35D2 | SERPINB6 |
| SLC38A2 | BACH1 |
| SLC38A2 | IFNGR1 |
| SLC38A2 | RAB1A |
| SLC39A13 | GNG11 |
| SLC39A13 | THBS1 |
| SLC44A3 | CD46 |
| SLTM | CCNT2 |
| SMAD4 | CAND1 |
| SMAD4 | EXOC5 |
| SMAD4 | NONO |
| SMARCC1 | MDM4 |
| SMARCC1 | MSL2 |
| SMC1A | LMNB1 |
| SMC2 | DHFR |
| SMC2 | KIF20A |
| SMC2 | KIF2C |
| SMC2 | ZNF273 |
| SMC3 | ADNP |
| SMC3 | PCNA |
| SMC3 | SRP9 |
| SMC6 | PIK3CA |
| SMCHD1 | CREB1 |
| SMEK1 | CTDSPL2 |
| SMG6 | DCTN1 |
| SMPD1 | CD63 |
| SMR3B | HSD3B2 |
| SNAI2 | INHBA |
| SNAI2 | MXRA7 |
| SNAP23 | ATP6V1C1 |
| SNHG7 | RBM14 |
| SNRNP70 | CRY2 |
| SNRPA | MCM2 |
| SNX13 | HRSP12 |
| SNX13 | RAB5A |
| SNX13 | TMEM126B |
| SNX2 | C20orf30 |
| SNX2 | COPS5 |
| SNX2 | DNM1L |
| SNX2 | LMAN1 |
| SNX2 | RAB1A |
| SNX33 | LMNA |
| SNX33 | RHOC |
| SNX33 | SERPINH1 |
| SNX7 | LARP6 |
| SNX7 | THBS1 |
| SOX10 | CDX1 |
| SOX21 | KIR2DL1 |
| SP100 | OAS1 |
| SPAG5 | ANP32E |
| SPAG5 | FEN1 |
| SPAG5 | NASP |
| SPAG5 | ZWINT |
| SPAG8 | AGER |
| SPAG8 | KSR1 |
| SPAG8 | LILRB5 |
| SPAG8 | POU6F2 |
| SPAG9 | HSPA8 |
| SPAST | EPB41 |
| SPINT1 | LRRC1 |
| SPINT1 | OSBPL2 |
| SPRED1 | PHLDA1 |
| SPTLC1 | C1orf56 |
| SPTLC1 | CD46 |
| SPTLC1 | DLAT |
| SPTLC1 | GNAI3 |
| SPTLC1 | HRSP12 |
| SPTLC1 | IARS2 |
| SPTLC1 | MED17 |
| SPTLC1 | MPZL1 |
| SPTLC1 | RIOK3 |
| SPTLC1 | TMED2 |
| SPTLC1 | TWF1 |
| SPTLC1 | UBC |
| SPTLC3 | TACR1 |
| SRPK1 | RPIA |
| SRPX | CALU |
| SRRM1 | CCNF |
| SRRM1 | CCNT1 |
| SRRM1 | CHAF1A |
| SRRM1 | KHSRP |
| SRRM1 | PDE4C |
| SRRM1 | PKMYT1 |
| SRRM1 | POLD1 |
| SRRM1 | RBM14 |
| SRRT | MXD3 |
| SRXN1 | SQSTM1 |
| SS18L2 | PPIH |
| SSBP1 | ECHDC1 |
| SSBP1 | HRSP12 |
| SSBP1 | NMD3 |
| SSBP1 | PSMA3 |
| SSBP1 | TBL1XR1 |
| SSTR4 | CSH2 |
| ST14 | TACSTD2 |
| ST5 | LAMC1 |
| ST5 | TIMP2 |
| ST6GALNAC2 | PRRG4 |
| STARD10 | TACSTD2 |
| STATH | ALDOB |
| STATH | PRB1 |
| STIL | CKS1B |
| STIL | TIMELESS |
| STIP1 | ERLIN1 |
| STIP1 | SNX13 |
| STIP1 | SSBP1 |
| STIP1 | STRAP |
| STMN1 | BIRC5 |
| STMN1 | CCNB2 |
| STMN1 | CENPA |
| STMN1 | CENPF |
| STMN1 | ESPL1 |
| STMN1 | GINS1 |
| STMN1 | GINS2 |
| STMN1 | HMGB1 |
| STMN1 | KIAA0101 |
| STMN1 | MCM7 |
| STMN1 | MYBL2 |
| STMN1 | NUDT1 |
| STMN1 | PLK1 |
| STMN1 | TIMELESS |
| STMN1 | TOP2A |
| STRAP | FASTKD2 |
| STRBP | EPB41 |
| STRBP | LUC7L2 |
| STRBP | MDM4 |
| STRBP | YEATS4 |
| STRBP | ZNF138 |
| STRBP | ZNF273 |
| STRBP | ZNF92 |
| STRN4 | ARFGAP1 |
| SUCLA2 | DLD |
| SUMO1 | ASAP1 |
| SUMO1 | RAB23 |
| SUPT5H | CRY2 |
| SUPT5H | FAM193B |
| SUPT5H | SNAPC4 |
| SUPT5H | USP20 |
| SURF4 | SLC39A7 |
| SURF4 | TMEM214 |
| SUV39H1 | AURKA |
| SUV39H1 | FOXM1 |
| SUV39H1 | MXD3 |
| SUV39H2 | RAD51 |
| SUZ12 | PABPN1 |
| SUZ12 | ZNF107 |
| SUZ12 | ZNF138 |
| SVIL | CAV2 |
| SYNJ1 | SRP9 |
| TAB2 | ATP6V1C1 |
| TAB2 | CLINT1 |
| TAB2 | CUL1 |
| TAB2 | MMADHC |
| TAB2 | MRPL13 |
| TAB2 | MSH2 |
| TAB2 | RPS6KB1 |
| TAB2 | UBE2H |
| TACC3 | AURKA |
| TACC3 | MYBL2 |
| TACC3 | RAD51AP1 |
| TACR1 | GPR68 |
| TACR1 | RAPSN |
| TACSTD2 | TMC5 |
| TAF12 | ARF1 |
| TAF12 | MARCH5 |
| TAF12 | PIP5K1A |
| TAF12 | SDHC |
| TAF5 | SP4 |
| TAF5 | TRA2B |
| TAF5 | YEATS4 |
| TAF9 | LMAN1 |
| TAOK1 | BRF1 |
| TAOK1 | PDE4C |
| TAOK1 | SF1 |
| TAOK1 | USF2 |
| TAOK1 | WDTC1 |
| TAOK2 | BRF1 |
| TAOK2 | PPP1R10 |
| TAP1 | RTP4 |
| TAPT1 | NAA38 |
| TBC1D10B | MEN1 |
| TC2N | EPHA1 |
| TCERG1 | HNRNPA3 |
| TCF3 | KDM2B |
| TCF3 | RBM14 |
| TCL1A | SIGLEC1 |
| TCL6 | CASS4 |
| TCL6 | CCR7 |
| TCL6 | LAT2 |
| TDP1 | NASP |
| TEAD3 | LMNA |
| TELO2 | PPP1R10 |
| TEX10 | POLR1B |
| TFCP2 | TAF12 |
| TFPI | ITGB1 |
| TGFBI | BCL9L |
| TGFBI | PLAT |
| THAP7 | ANAPC2 |
| THBS1 | KIF13A |
| THBS1 | LMNA |
| THBS1 | SERPINB7 |
| THBS1 | TRIM16 |
| THOP1 | DDX51 |
| THOP1 | MCM2 |
| TIA1 | SRP9 |
| TIA1 | ZNF184 |
| TJP1 | DSP |
| TJP1 | LAMA5 |
| TJP3 | CNKSR1 |
| TJP3 | EVPL |
| TJP3 | F11R |
| TJP3 | FAM83B |
| TJP3 | PFKFB2 |
| TJP3 | SMPDL3B |
| TJP3 | ST14 |
| TK1 | RAD51 |
| TLCD1 | CGN |
| TLCD1 | EFNA1 |
| TLCD1 | ELF3 |
| TLCD1 | TSPAN1 |
| TLN1 | ARHGEF1 |
| TM9SF2 | AZIN1 |
| TM9SF2 | CD46 |
| TM9SF2 | LMBRD1 |
| TM9SF2 | PSEN1 |
| TM9SF2 | RALB |
| TM9SF2 | TTC35 |
| TM9SF2 | UGP2 |
| TMCO3 | CD46 |
| TMCO3 | TBL1XR1 |
| TMED2 | C19orf2 |
| TMED2 | CUL4B |
| TMED2 | GPR89B |
| TMEM125 | GLS2 |
| TMEM135 | HNRNPF |
| TMEM158 | MXRA7 |
| TMEM158 | RASSF8 |
| TMEM165 | UBC |
| TMEM184A | ARHGEF16 |
| TMEM184B | NBL1 |
| TMEM194A | CKS1B |
| TMEM30A | CD46 |
| TMEM30B | CXCL16 |
| TMEM30B | IRF6 |
| TMEM43 | RAB11FIP5 |
| TMEM45B | CGN |
| TMEM45B | PLS1 |
| TMEM51 | TNFRSF21 |
| TMPRSS4 | ALS2CL |
| TMPRSS4 | LAD1 |
| TMPRSS4 | S100A14 |
| TMPRSS6 | B4GALNT3 |
| TMPRSS6 | CD6 |
| TMPRSS6 | LILRB3 |
| TMPRSS6 | OR8B8 |
| TNFAIP1 | ITGAV |
| TNFAIP3 | IKBKE |
| TNFAIP3 | NFKBIA |
| TNFAIP3 | STK10 |
| TNFRSF12A | CDC42EP2 |
| TNFRSF12A | ELOVL1 |
| TNFRSF12A | RPS6KA4 |
| TNFSF15 | IRF6 |
| TNIP1 | PSMB8 |
| TNK1 | CGN |
| TNK1 | DSG2 |
| TNK1 | GOLT1A |
| TNK1 | INADL |
| TNK1 | SERPINB5 |
| TNK2 | CABIN1 |
| TNK2 | GTF2H3 |
| TNPO2 | DCTN1 |
| TNR | CD6 |
| TNS4 | GJB3 |
| TNS4 | ITGB6 |
| TNS4 | TTC22 |
| TOM1L2 | LMNA |
| TOMM22 | HNRNPH1 |
| TOMM22 | PSMC3 |
| TOP2A | ANP32E |
| TOP2A | CENPF |
| TOP2B | RPIA |
| TPM1 | DSTN |
| TPM1 | LOXL2 |
| TPM1 | RIN2 |
| TRA2B | DDX46 |
| TRAF3IP3 | LCP2 |
| TRAF3IP3 | NKG7 |
| TRIM23 | GLUD2 |
| TRIM49 | F13B |
| TRIOBP | PRSS23 |
| TRIOBP | SSH3 |
| TRIOBP | TNFRSF1A |
| TRIP6 | RRAS |
| TRMT5 | H2AFV |
| TRMT5 | PCNA |
| TRMT61A | CLCN7 |
| TSPAN1 | EVPL |
| TSPAN1 | KRT80 |
| TSPAN1 | SEMA4B |
| TSPAN1 | SERPINB5 |
| TSPAN4 | DAB2 |
| TSPAN4 | RAB11FIP5 |
| TSPAN4 | SERPINE1 |
| TSPO | FOSL1 |
| TSSK3 | CEACAM3 |
| TSSK3 | GRIN1 |
| TTK | HMGB2 |
| TTK | MCM7 |
| TTK | NEIL3 |
| TUBA1B | GINS1 |
| TUBA1B | NCAPG |
| TUBB | TUBA1B |
| TUBB3 | PCBP4 |
| TUBE1 | ASNS |
| TYMS | BARD1 |
| TYMS | CCNF |
| TYMS | HMGB1 |
| UBA1 | HCFC1 |
| UBA7 | RTP4 |
| UBE2H | ATP6V1C1 |
| UBE2H | PEX2 |
| UBE2H | RNF11 |
| UBE2H | TMEM59 |
| UBE2H | YWHAZ |
| UBE2N | CNBP |
| UBE2N | FUS |
| UBTD1 | MMP14 |
| UBTD1 | PRSS23 |
| UBXN6 | CUL7 |
| UGCG | SRGAP1 |
| UGP2 | B3GNT1 |
| UGP2 | MGAT2 |
| UGP2 | PSMA3 |
| UQCRC2 | FH |
| USO1 | DNAJC10 |
| USP1 | MSH2 |
| VAMP3 | PYGO2 |
| VCL | RAI14 |
| VCL | RIN2 |
| VCL | S100A2 |
| VCL | SAMD4A |
| VCL | TWSG1 |
| VEGFC | AOX1 |
| VEGFC | CRIM1 |
| VEGFC | DFNA5 |
| VEGFC | INHBA |
| VPRBP | DDX55 |
| VPS26A | MYL12B |
| VPS4A | COBRA1 |
| VPS4A | UBA1 |
| VWA3A | MS4A6A |
| WAS | ADRBK1 |
| WAS | MS4A6A |
| WDR43 | IPO4 |
| WDR61 | RNF146 |
| WDR62 | E2F1 |
| WDR62 | PKMYT1 |
| WDR7 | PHF21A |
| WDR76 | KIF2C |
| WDR76 | MATR3 |
| WDR76 | MCM2 |
| WDR76 | MYBL2 |
| WDR76 | TRA2B |
| WHSC1 | EZH2 |
| WNT7B | KRT7 |
| WNT7B | KRT80 |
| WWTR1 | PEA15 |
| WWTR1 | PRSS23 |
| XAB2 | E4F1 |
| XPO7 | CREB1 |
| XPO7 | DLAT |
| XPO7 | FH |
| XPO7 | HSPD1 |
| XPO7 | MED17 |
| XPO7 | POLR2K |
| XPO7 | RAD17 |
| XPO7 | RBM12 |
| XPO7 | UBXN4 |
| XRCC2 | PGF |
| XRCC3 | MYBL2 |
| YEATS4 | NACAP1 |
| YIPF4 | SPTLC1 |
| YIPF5 | CD164 |
| YIPF5 | RAB23 |
| YPEL1 | TIA1 |
| YWHAH | MRPL42 |
| YWHAH | UGP2 |
| YWHAZ | CREB1 |
| YWHAZ | UGP2 |
| ZBED4 | ATP6V0A2 |
| ZBED4 | DFFB |
| ZBED4 | NASP |
| ZBTB11 | CCDC76 |
| ZBTB11 | RNF146 |
| ZBTB39 | ZCCHC3 |
| ZBTB44 | HNRNPA1 |
| ZBTB44 | RBM39 |
| ZBTB48 | CCNT1 |
| ZBTB48 | SF3A2 |
| ZBTB6 | MSH2 |
| ZBTB7A | TAPBP |
| ZC3H4 | RBM14 |
| ZC3H7B | COBRA1 |
| ZC3H7B | FSCN2 |
| ZC3H7B | LTB4R |
| ZC3H7B | POLH |
| ZC3H7B | USP36 |
| ZCCHC4 | HNRNPC |
| ZCCHC8 | TCERG1 |
| ZDHHC7 | CD151 |
| ZDHHC7 | YAP1 |
| ZEB2 | GNB4 |
| ZFYVE21 | ATP8B1 |
| ZFYVE21 | UBE2H |
| ZMYM2 | CCNT2 |
| ZNF107 | MTF2 |
| ZNF107 | TMPO |
| ZNF207 | PSMC3 |
| ZNF207 | XRCC5 |
| ZNF227 | EXOC5 |
| ZNF227 | TMEM126B |
| ZNF248 | TIA1 |
| ZNF273 | E2F2 |
| ZNF273 | MTF2 |
| ZNF274 | ZNF75A |
| ZNF358 | CDC42BPB |
| ZNF385D | SEMG2 |
| ZNF385D | TRPC7 |
| ZNF407 | ATP2B3 |
| ZNF407 | NACA2 |
| ZNF500 | HCFC1 |
| ZNF580 | SF1 |
| ZNF589 | POLR1B |
| ZNF611 | HAUS2 |
| ZNF654 | EXOC5 |
| ZNF670 | RNF138 |
| ZNF700 | ZNF107 |
| ZNF711 | KIF1A |
| ZNF768 | RFNG |
| ZNF780A | CNOT8 |
| ZNF780A | HNRNPF |
| ZNF780A | MSH2 |
| ZNF780A | PSMD10 |
| ZNF780A | UBC |
| ZNF84 | ZFP14 |
| ZWILCH | DONSON |
| ZWINT | CDC20 |
| ZWINT | DCK |
| ZWINT | SGOL1 |
| ZWINT | STIL |
| ZWINT | UBE2C |
| ZXDC | MDM4 |
| AGPAT9 | ASPH |
| ANAPC10 | HRSP12 |
| ACTN4 | KDELR3 |
| ANP32A | MCM7 |
| ANKFY1 | TFG |
| ANXA5 | ANTXR1 |
| ARPC1A | KLHL12 |
| ATG5 | UBC |
| BLVRB | SSH3 |
| CA6 | CD6 |
| CBLC | HOOK2 |
| C8A | SPTLC3 |
| CBLC | SSH3 |
| CDC25A | CENPM |
| CDC25A | DHFR |
| CDCA8 | FAM64A |
| CD52 | FCER2 |
| CD151 | MET |
| CCNC | SCYL2 |
| CEACAM6 | ST14 |
| CYR61 | CARD10 |
| CYR61 | CDC42EP1 |
| CNOT3 | DDX6 |
| CXXC1 | DNASE1L2 |
| CYP2S1 | IL18 |
| DAG1 | KDELR3 |
| CYR61 | LIF |
| CSNK1G2 | MAPK8IP3 |
| DAG1 | MGAT4B |
| CXXC1 | POGZ |
| CWF19L1 | POLR2D |
| CYR61 | PTRF |
| CYR61 | SLC12A4 |
| CNOT3 | SMARCB1 |
| CYR61 | TNFAIP1 |
| DEPDC1 | AURKB |
| DHX32 | BCAR3 |
| DDX28 | GGA2 |
| EBNA1BP2 | HSPA4 |
| ENO1 | B3GNT1 |
| EPHA2 | CCND1 |
| ENO1 | CD46 |
| ESPN | CDH1 |
| EPS8L1 | CXCL16 |
| ERRFI1 | FOSL1 |
| ENO1 | HRSP12 |
| EMP3 | LGALS1 |
| FAM193A | RPRD2 |
| EPB41 | SAFB |
| EPHA2 | SSH3 |
| FBXO46 | CARM1 |
| FGR | CD48 |
| FCER2 | CR1 |
| FBXO46 | CSNK1G2 |
| FDX1 | GDE1 |
| FLII | PLEC |
| FBXO46 | TCF20 |
| FBL | TCF3 |
| FBXO31 | ZNF500 |
| GLB1 | ATP6V0E1 |
| GNG12 | CUEDC1 |
| GNG12 | DKK3 |
| GIN1 | ITCH |
| GTPBP1 | MAPK8IP3 |
| GTF2A2 | PSMA2 |
| GBP1 | PTRF |
| GNG12 | PTRF |
| GDI2 | RHOA |
| GDI2 | RIOK3 |
| GDI2 | RPP14 |
| GNG12 | SMURF2 |
| GSTO2 | TACSTD2 |
| GUCA2B | TCL1A |
| GBP1 | TRIM22 |
| HNRNPCL1 | ABT1 |
| IFRD2 | GEMIN5 |
| HERC6 | PARP14 |
| IFNA6 | PPP1R3A |
| HERC6 | STAT1 |
| ILK | DLGAP4 |
| ITGB3BP | DNMT1 |
| KHDRBS1 | DNMT1 |
| KIAA1522 | EVPL |
| KIAA1522 | GPR56 |
| IRF2 | HLA-B |
| KIAA1522 | PRRG4 |
| ITPKC | SSH3 |
| KIF2C | AURKB |
| LEPRE1 | GNAI2 |
| MBD3 | UBN1 |
| MRPS15 | CAPRIN1 |
| MPP7 | CDS1 |
| MTF2 | DNMT1 |
| MTA1 | GTF2H3 |
| MRPL37 | POLR2E |
| MUTYH | POU2F1 |
| MRPS15 | VDAC1 |
| NBR1 | ARPC5 |
| OR2J3 | KRT76 |
| PDE6C | APOB |
| PERP | CAST |
| PLK4 | CENPL |
| POLD1 | CSNK1G2 |
| PLK4 | DHX9 |
| PDCD11 | FARSA |
| PLOD1 | HTRA1 |
| PICALM | MAPRE1 |
| POLD1 | POLR2A |
| POLD1 | SF3A2 |
| POLD1 | THOP1 |
| PLA2G2F | TNP2 |
| PDE12 | TRPC7 |
| RAD17 | ARPC5 |
| PSEN1 | B3GNT2 |
| PRPF18 | GIN1 |
| PRPF18 | RIOK3 |
| PVRL2 | SSH3 |
| PRRG2 | ST14 |
| PRRG2 | STARD10 |
| RAD17 | TBL1XR1 |
| RAD23B | TMED2 |
| RAD23B | UBC |
| RHOC | AHNAK |
| RBM7 | ARFGEF1 |
| RAX2 | FAM71A |
| RRAS | KDELR3 |
| RHOA | MSH6 |
| RCC1 | PHF5A |
| RCC1 | PPAN |
| RHOC | PTRF |
| RPS3 | RPL30 |
| SFN | ELMO3 |
| SERTAD1 | KDELR3 |
| SGSM3 | MAPK8IP3 |
| SDHB | MRPL13 |
| SDHD | MRPL13 |
| SFN | OVOL1 |
| SFN | P2RY2 |
| SFN | RASSF7 |
| SFN | SP6 |
| SH3BGRL3 | TGFB1I1 |
| SFN | UGT1A1 |
| SMARCB1 | CCNF |
| SRRM1 | CHERP |
| SULT2B1 | CRB3 |
| STIL | DNMT1 |
| SULT2B1 | ELMO3 |
| SRRM1 | GMIP |
| SULT2B1 | ST14 |
| TACSTD2 | ATP2C2 |
| TMC4 | CXCL16 |
| TCF21 | DSPP |
| TACSTD2 | EHF |
| TMC4 | ESRP2 |
| TACSTD2 | F2RL1 |
| TACSTD2 | FRK |
| TAF12 | HNRNPF |
| TJP1 | PTK2 |
| TMC4 | SH2D3A |
| TFG | SLC30A5 |
| SYTL1 | ST14 |
| TACSTD2 | ST14 |
| SYTL1 | STXBP2 |
| SYCP1 | TPSAB1 |
| TACSTD2 | TSPAN15 |
| TRAIP | ADSL |
| TRMU | CCNF |
| TOMM22 | CCT2 |
| TRAIP | CENPM |
| TYK2 | CUL9 |
| TRPC7 | FCGR3B |
| TYK2 | GGA3 |
| TSPAN1 | GPR56 |
| TMEM39B | KHSRP |
| TOE1 | KHSRP |
| TXLNA | KHSRP |
| TMEM39B | MBD3 |
| TXLNA | MBD3 |
| TTK | NUDT1 |
| TSPAN1 | PRRG4 |
| TRIM29 | PTK6 |
| TRIM23 | RAB1A |
| TRIM29 | RAB25 |
| TRPM4 | SSH3 |
| TMPRSS4 | ST6GALNAC1 |
| TRAIP | TROAP |
| TRIOBP | ZNFX1 |
| UBR4 | ARFGAP1 |
| VEGFC | CAPN2 |
| WHSC1 | CDCA3 |
| ZBTB16 | CSH2 |
| ZBTB16 | FCGR3A |
| ZBTB6 | H3F3C |
| ZBTB6 | ILF2 |
| YWHAE | KLHL12 |
| YAP1 | LAMB3 |
| VIPR1 | MARVELD2 |
| WDHD1 | MCM6 |
| YWHAH | MED21 |
| YAP1 | PTPN14 |
| YTHDC1 | RBM39 |
| USO1 | RPAP3 |
| VEGFC | TFPI2 |
| VAMP3 | TGFB1I1 |
| WDHD1 | TPX2 |
| ZBTB48 | ZNF335 |
| ZBTB17 | ZNF668 |
| ZCCHC24 | CALD1 |
| ZCCHC7 | CPSF6 |
| ZC3H7B | CUL9 |
| ZC3H7B | ERN2 |
| ZNF407 | MEOX2 |
| ZC3H7B | MUTYH |
| ZNF593 | MYBBP1A |
| ZC3H7B | RNF40 |
| ZWINT | SKP2 |

TABLE 2
SDL network which comprises the gene pairs listed.
When gene A is over-active gene B is essential

| Gene A | Gene B |
|---|---|
| A2M | A2M |
| AASDH | AASDH |
| ABCB1 | ABCB4 |
| ABCB8 | FASTK |
| ABCC3 | ABCC3 |
| ABCC3 | GPRC5C |
| ABCC3 | ITGB4 |
| ABCF1 | MDC1 |
| ABCF3 | NRBP1 |
| ABHD13 | CUL4A |
| ABI1 | MLLT10 |
| ABLIM3 | P4HA2 |
| ABO | ORM1 |
| ABT1 | MAPK14 |
| ACADVL | MINK1 |
| ACBD6 | ACBD6 |
| ACHE | ACHE |
| ACIN1 | BRF1 |
| ACOT8 | ACOT8 |
| ACP1 | B3GNT2 |
| ACP1 | PIGF |
| ACP2 | ACP2 |
| ACTN1 | ACTN1 |
| ACTN4 | ETHE1 |
| ACTN4 | NCEH1 |
| ACTR3B | ACTR3B |
| ACTR3C | CLDN4 |
| ACYP1 | UBR7 |
| ADAM9 | ATP6V1C1 |
| ADAM9 | CTSA |
| ADAMTS5 | ADAMTS5 |
| ADAMTSL4 | ADAMTSL4 |
| ADAP1 | KLF5 |
| ADAR | TARS2 |
| ADAT3 | RNF126 |
| ADCK1 | ADCK1 |
| ADD1 | ADD1 |
| ADI1 | ADI1 |
| ADNP | ADNP |
| ADNP | CCNT2 |
| ADRA1D | FKBP8 |
| ADRB3 | ADRB3 |
| ASDL | DRG1 |
| ADSS | IARS2 |
| AFF4 | TMED2 |
| AGGF1 | TAF9 |
| AGPAT3 | AGPAT3 |
| AGPAT5 | KIAA1967 |
| AGR2 | CLDN4 |
| AGRN | GJB3 |
| AGTR1 | AGTR1 |
| AHR | MET |
| AIF1 | FGD2 |
| AIF1 | GUCA1A |
| AIF1 | HLA-DOA |
| AIMP1 | RAP1GDS1 |
| AIMP1 | SRP72 |
| AIMP2 | MRPS17 |
| AK1 | NCS1 |
| AKAP11 | FAM48A |
| AKAP8 | ELL |
| AKAP8 | GTPBP3 |
| AKAP8 | RAB8A |
| AKAP8L | AKAP8L |
| AKAP8L | UPF1 |
| AKAP9 | PEX1 |
| AKNA | AKNA |
| AKR1A1 | AKR1A1 |
| AKT1S1 | AP2S1 |
| AKTIP | AKTIP |
| AKTIP | ITFG1 |
| ALDH3B1 | ALDH3B1 |
| ALKBH4 | TRRAP |
| ALPK3 | ALPK3 |
| AMDHD2 | MPG |
| AMDHD2 | STUB1 |
| AMDHD2 | TMEM8A |
| AMIGO2 | EGFR |
| AMOTL2 | B4GALT4 |
| AMOTL2 | OSMR |
| AMOTL2 | TM4SF1 |
| AMZ2 | KLHL12 |
| ANAPC11 | STRA13 |
| ANAPC2 | GTF3C5 |
| ANAPC2 | MRPS2 |
| ANAPC7 | TMPO |
| ANGEL2 | ARID4B |
| ANKFY1 | RPS6KB1 |
| ANKRD16 | ANKRD16 |
| ANKRD16 | NUDT5 |
| ANKRD16 | SUV39H2 |
| ANLN | MET |
| ANO1 | CAPN1 |
| ANO1 | S100A14 |
| ANP32A | CLPX |
| ANP32A | PIAS1 |
| ANP32B | C9orf80 |
| ANP32B | POLE3 |
| ANP32B | STRBP |
| ANP32E | ANP32E |
| ANP32E | LIN9 |
| ANPEP | ANPEP |
| ANXA2 | ALDH1A3 |
| ANXA9 | ELF3 |
| ANXA9 | EVPL |
| ANXA9 | PRSS22 |
| AP1M1 | PIN1 |
| AP2A1 | NUCB1 |
| AP2M1 | CYB5R3 |
| AP2S1 | AP2S1 |
| AP3B1 | GDE1 |
| AP3B1 | GIN1 |
| AF3B1 | SNX2 |
| AP3B1 | TAF9 |
| AP3M2 | POLB |
| API5 | CAPRIN1 |
| APPL2 | SCYL2 |
| APTX | NDUFB6 |
| ARF1 | ADIPOR1 |
| ARF1 | MRPL13 |
| ARF1 | YWHAZ |
| ARF3 | ARF3 |
| ARF4 | RPP14 |
| ARFGAP2 | SF1 |
| ARFGEF1 | ARPC5 |
| ARFGEF1 | AZIN1 |
| ARFGEF1 | MAPRE1 |
| ARFGEF1 | NCOA2 |
| ARFGEF1 | PSMD12 |
| ARFGEF1 | TCEB1 |
| ARGLU1 | PDS5B |
| ARHGAP23 | ARHGAP23 |
| ARHGAP29 | EGFR |
| ARHGAP29 | F3 |
| ARHGAP33 | LIG1 |
| ARHGEF5 | TMEM139 |
| ARID1A | HNRNPR |
| ARID1A | NASP |
| ARID1B | BCLAF1 |
| ARID1B | FBXO5 |
| ARID2 | ZBTB39 |
| ARIH2 | PDE12 |
| ARL1 | GDE1 |
| ARL3 | ACTR1A |
| ARL6IP4 | OGFOD2 |
| ARL8B | EDEM1 |
| ARMC1 | MAPRE1 |
| ARMC1 | YWHAZ |
| ARMC10 | DUS4L |
| ARMC6 | ATP13A1 |
| ARMC6 | FARSA |
| ARMC6 | RAVER1 |
| ARMC8 | SNX4 |
| ARNT | ARNT |
| ARRB1 | ARRB1 |
| ARRB2 | G5G2 |
| ARRDC1 | BSPRY |
| ARVCF | ARVCF |
| ASAP1 | ARPC5 |
| ASAP1 | CLTC |
| ASAP1 | HRSP12 |
| ASAP1 | MRPS28 |
| ASAP1 | PEX2 |
| ASAP1 | PRKAR1A |
| ASAP1 | TCEB1 |
| ASF1A | KATNA1 |
| ASF1A | PCMT1 |
| ASF1B | RAVER1 |
| ASL | ASL |
| ASPH | PLEC |
| ASPH | S100A2 |
| ASPM | NEK2 |
| ASPSCR1 | MRPL38 |
| ATAD2 | CCNE2 |
| ATAD2 | CDC5L |
| ATAD2 | KIF14 |
| ATAD2 | MAPRE1 |
| ATAD2 | MCM3 |
| ATAD2 | MCM4 |
| ATAD2 | PCNA |
| ATAD2 | RFC4 |
| ATAD2 | TOP2A |
| ATAD2 | TOPBP1 |
| ATAD2 | WDR67 |
| ATE1 | NSMCE4A |
| ATF6B | TAPBP |
| ATG2A | MAP3K11 |
| ATG2A | PEX16 |
| ATG3 | IL20RB |
| ATG4C | PRPF38A |
| ATMIN | MBTPS1 |
| ATP1B1 | ELF3 |
| ATP1B3 | ATP1B3 |
| ATP4A | ATP4A |
| ATP5A1 | HDHD2 |
| ATP5A1 | TXNL1 |
| ATP5C1 | NUDT5 |
| ATP5C1 | SUV39H2 |
| ATP5D | NCLN |
| ATP5D | RNF126 |
| ATP5F1 | MRPL37 |
| ATP5L | SLC37A4 |
| ATP5SL | BCKDHA |
| ATP6V0C | ATP6V0D1 |
| ATP6V0E1 | MGAT4B |
| ATP6V1B1 | ATP6V1B1 |
| ATP6V1C1 | IARS2 |
| ATRIP | PDE12 |
| ATRN | POLR3F |
| ATXN2L | PRR14 |
| ATXN2L | ZNF646 |
| ATXN3 | PRPF39 |
| AURKA | ECT2 |
| AUTS2 | AUTS2 |
| AVPI1 | BAG3 |
| AZI1 | CUL9 |
| AZI1 | NUP85 |
| AZI2 | DYNC1LI1 |
| AZIN1 | HRSP12 |
| AZIN1 | TCEB1 |
| B3GALT2 | B3GALT2 |
| B3GAT3 | B3GAT3 |
| B4GALNT3 | DEFB118 |
| B4GALT3 | USP21 |
| BAG4 | ASH2L |
| BAZ1A | PRPF39 |
| BCAR3 | BCAR3 |
| BCAR3 | LEPROT |
| BCAS2 | ILF2 |
| BCKDK | AMDHD2 |
| BCKDK | BCKDK |
| BCKDK | STUB1 |
| BCKDK | VKORC1 |
| BCL2 | BCL2 |
| BCL2L1 | BCL2L1 |
| BCL2L1 | KRT8 |
| BCLAF1 | ADAT2 |
| BCMO1 | BCMO1 |
| BDP1 | BDP1 |
| BDP1 | CHD1 |
| BHLHE41 | BHLHE41 |
| BICD1 | BICD1 |
| BIRC2 | YAP1 |
| BIRC5 | AURKA |
| BIRC5 | RRM2 |
| BLVR8 | LPP |
| BMP2 | BMP2 |
| BPTF | COIL |
| BPTF | MED13 |
| BPTF | ZNF652 |
| BRAP | MAPKAPK5 |
| BRCA2 | BRCA2 |
| BRD2 | EHMT2 |
| BRD2 | PBX2 |
| BRD3 | BRD3 |
| BRD4 | PRKCSH |
| BRD4 | SIN3B |
| BRD7 | RFWD3 |
| BRE | BRE |
| BRF1 | ENTPD5 |
| BRF1 | PPP1R10 |
| BRF2 | GOLGA7 |
| BRIX1 | RAD1 |
| BRIX1 | TRIP13 |
| BRMS1 | RAB18 |
| BRPF1 | SGOL1 |
| BSPRY | ENTPD2 |
| BTBD2 | DNM2 |
| BTBD2 | MBD3 |
| BTF3 | CHD1 |
| BTF3 | TAF9 |
| BTG2 | RGS16 |
| BTN2A1 | BTN2A2 |
| BTN2A2 | BTN2A2 |
| BTN3A1 | BTN3A2 |
| BTN3A1 | HLA-F |
| BUB1B | AQR |
| BUB1B | C15orf23 |
| BUB3 | KIF20B |
| BUB3 | NSMCE4A |
| BUD13 | ATP5L |
| BUD13 | HINFP |
| BUD13 | MLL |
| C10orf137 | TAF5 |
| C10orf47 | C10orf47 |
| C11orf16 | C11orf16 |
| C11orf2 | MEN1 |
| C11orf48 | POLR2G |
| C11orf48 | PRPF19 |
| C11orf57 | CUL5 |
| C11orf68 | CD59 |
| C11orf92 | C11orf92 |
| C12orf29 | CCDC59 |
| C12orf47 | MAPKAPK5 |
| C12orf65 | MPHOSPH9 |
| C12orf73 | ALKBH2 |
| C14orf1 | C14orf1 |
| C14orf102 | RCOR1 |
| C14orf102 | YY1 |
| C14orf119 | LRP10 |
| C14orf129 | GOLGA5 |
| C14orf142 | UBR7 |
| C14orf156 | CDKN3 |
| C14orf156 | EXOC5 |
| C14orf166 | MNAT1 |
| C14orf2 | PAPOLA |
| C15orf42 | DUT |
| C15orf44 | CLPX |
| C16orf42 | AMDHD2 |
| C16orf42 | CD2BP2 |
| C16orf42 | PMM2 |
| C16orf45 | C16orf45 |
| C16orf57 | C16orf57 |
| C16orf79 | E4F1 |
| C16orf80 | CIAPIN1 |
| C17orf48 | ZNF18 |
| C17orf51 | C17or51 |
| C17orf62 | FMNL1 |
| C17orf70 | MAP3K3 |
| C17orf80 | DDX42 |
| C17orf80 | RAD51C |
| C17orf81 | GSG2 |
| C18orf21 | TXNL1 |
| C19orf24 | RNF126 |
| C19orf29 | RNF126 |
| C19orf33 | EPS8L1 |
| C19orf40 | SYMPK |
| C19orf43 | FARSA |
| C19orf48 | BCL2L12 |
| C19orf48 | LIG1 |
| C19orf53 | NDUFB7 |
| C19orf6 | RNF126 |
| C1QBP | GSG2 |
| C1QTNF3 | C1QTNF3 |
| C1QTNF9 | C1QTNF9 |
| C1R | C1R |
| C1orf112 | CENPF |
| C1orf112 | RFC4 |
| C1orf112 | SKP2 |
| C1orf112 | TOPBP1 |
| C1orf210 | INADL |
| C1orf210 | TACSTO2 |
| C1orf27 | HRSPF12 |
| C1orf27 | KLHL12 |
| C1orf43 | DAP3 |
| C1orf43 | NDUFS2 |
| C1orf63 | CCNL2 |
| C20orf166 | H1FNT |
| C20orf30 | MOCS3 |
| C21orf2 | DIP2A |
| C2CD2L | HMB5 |
| C2orf28 | PDIA6 |
| C4orf27 | NEK1 |
| C5orf22 | RAD1 |
| C5orf54 | TRIM23 |
| C6orf106 | C6orf105 |
| C6orf115 | REPS1 |
| C6orf132 | ANXA9 |
| C6orf132 | S100A14 |
| C6orf132 | TEAD3 |
| C6orf136 | C6orf136 |
| C6orf162 | ADAT2 |
| C7orf23 | C7orf23 |
| C7orf26 | POM121 |
| C7orf42 | TYW1 |
| C7orf50 | RAC1 |
| C8orf38 | CCNE2 |
| C8orf76 | DSCC1 |
| C8orf76 | WDR67 |
| C9orf23 | SIGMAR1 |
| C9orf43 | C9orf43 |
| C9orf46 | JAK2 |
| C9orf78 | POLE3 |
| C9orf80 | PDCL |
| C9orf86 | MRPS2 |
| CA4 | KCNH6 |
| CABIN1 | GGA1 |
| CABIN1 | PLA2G6 |
| CALML4 | CALML4 |
| CAMK2N1 | PTPRF |
| CAP2 | CFL2 |
| CAPN1 | BRMS1 |
| CAPN1 | CST6 |
| CAPN1 | MAP3K11 |
| CAPN1 | NADSYN1 |
| CAPN1 | OTUB1 |
| CAPN7 | LSM3 |
| CAPRIN1 | CKAP5 |
| CAPS2 | CAPS2 |
| CARS2 | TFDP1 |
| CASC3 | NKIRAS2 |
| CASP2 | EZH2 |
| CASP2 | LUC7L2 |
| CASP2 | ZNF212 |
| CASP2 | ZNF282 |
| CASP8AP2 | HDAC2 |
| CASP8AP2 | SENP6 |
| CASQ1 | OR10J1 |
| CASS4 | CASS4 |
| CAV1 | ANLN |
| CAV1 | FAM20C |
| CAV1 | STEAP1 |
| CAV2 | INHBA |
| CBL | BUD13 |
| CBL | CBL |
| CBLC | MYH14 |
| CBLL1 | SLC25A40 |
| CBX2 | CBX2 |
| CC2D1A | HAMP |
| CC2D1A | RAD23A |
| CC2D1A | ZNF787 |
| CCAR1 | ZNF37A |
| CCDC101 | PRR14 |
| CCDC117 | MSL2 |
| CCDC124 | FARSA |
| CCDC130 | CC2D1A |
| CCDC130 | STRN4 |
| CCDC22 | PQBP1 |
| CCDC90A | NUP153 |
| CCDC94 | MBD3 |
| CCNA2 | CENPE |
| CCNA2 | MAD2L1 |
| CCNB1 | CCNB1 |
| CCNB1 | CDC25C |
| CCNB1IP1 | C14orf93 |
| CCNE1 | CCNE1 |
| CCNE2 | CCNE2 |
| CCNE2 | GMNN |
| CCNE2 | MCM3 |
| CCNE2 | TCF19 |
| CCNE2 | TOP2A |
| CCNF | E4F1 |
| CCNH | PTCD2 |
| CCNH | RARS |
| CCNH | SNX2 |
| CCNH | TAF9 |
| CCNI | CCNI |
| CCNJL | CCNJL |
| CCNL1 | MBD4 |
| CCNL1 | TBL1XR1 |
| CCT2 | TFCP2 |
| CCT3 | USP21 |
| CCT4 | PRKRA |
| CCT4 | SSB |
| CCT5 | PSMD12 |
| CD46 | ADIPOR1 |
| CD46 | ELF3 |
| CD46 | FZD6 |
| CD46 | PTK2 |
| CD46 | SRP9 |
| CD48 | TNFAIP8L2 |
| CD72 | CD72 |
| CD83 | CD83 |
| CDC20 | RAD54L |
| CDC20 | STIL |
| CDC27 | UTP18 |
| CDC37 | FARSA |
| CDC37L1 | JAK2 |
| CDC42SE2 | CDC42SE2 |
| CDC45 | BUB1 |
| CDC45 | POLQ |
| CDC5L | NMD3 |
| CDC6 | SPAG5 |
| CDC7 | PTBP2 |
| CDC7 | STIL |
| CDCA8 | CDC7 |
| CDCA8 | FAF1 |
| CDCA8 | ITGB3BP |
| COCA8 | POLQ |
| CDCA8 | PPIH |
| CDK1 | HNRNPF |
| CDK17 | SCYL2 |
| CDK5RAP1 | C20orf4 |
| CDK5RAP1 | DHX35 |
| CDK6 | CDK6 |
| CDK7 | GDE1 |
| CDK7 | RARS |
| CDK7 | TAF9 |
| CDK7 | XRCC4 |
| CDK9 | FPGS |
| CDKAL1 | MDC1 |
| CDKN2D | CDKN2D |
| CDKN3 | TRMT5 |
| CDKN3 | VRK1 |
| CDS1 | BTC |
| CDS1 | SHROOM3 |
| CDSN | AIF1 |
| CDSN | CDSN |
| CDX2 | CDX2 |
| CEBPB | CEBPB |
| CEBPE | CEBPE |
| CECR5 | POLR1B |
| CELF3 | CYP11B2 |
| CELF3 | HRH3 |
| CENPA | CENPO |
| CENPA | ECT2 |
| CENPC1 | ELF2 |
| CENPC1 | LARP7 |
| CENPC1 | LSM6 |
| CENPC1 | MAD2L1 |
| CENPF | MDC1 |
| CENPM | L3MBTL2 |
| CENPN | C16orf61 |
| CENPT | TAF1C |
| CEP192 | THOC1 |
| CEP350 | MDM4 |
| CEP55 | KIF20B |
| CEP57 | BUD13 |
| CEP57 | CHEK1 |
| CEP63 | CEP63 |
| CEP76 | MSH2 |
| CERK | CERK |
| CES2 | CES2 |
| CETN3 | TAP9 |
| CFL1 | FAM89B |
| CFL2 | PRKD1 |
| CFL2 | PTPN21 |
| CGGBP1 | CGGBP1 |
| CGGBP1 | NR2C2 |
| CGN | ELF3 |
| CHAF1A | PIN1 |
| CHAF1A | SLC39A3 |
| CHCHD1 | HSPA14 |
| CHCHD3 | PAXIP1 |
| CHD1 | BDP1 |
| CHD1 | CHD1 |
| CHERP | AKAP8 |
| CHERP | ATP13A1 |
| CHERP | CNOT3 |
| CHERP | FARSA |
| CHERP | GTPBP3 |
| CHERP | TNPO2 |
| CHERP | UPF1 |
| CHMP4C | IRF6 |
| CHMP5 | NMD3 |
| CHMP5 | SENP2 |
| CHMP7 | CNOT7 |
| CHMP7 | ELP3 |
| CHPF2 | CHPF2 |
| CHRNA5 | PTPLAD1 |
| CIAPIN1 | COX4NB |
| CIC | IRF2BP1 |
| CIC | MARK4 |
| CISD1 | CISD1 |
| CKAP2 | BRCA2 |
| CKAP5 | CELF1 |
| CLCF1 | CDC42EP2 |
| CLCN7 | RNF40 |
| CLCN7 | ZNF500 |
| CLDN1 | ABCC3 |
| CLDN1 | ITGB4 |
| CLDN3 | CLDN4 |
| CLDN4 | SLPI |
| CLDND1 | DLG1 |
| CLDND1 | SLC3SA5 |
| CLIC3 | PTGES |
| CLIC4 | TAF12 |
| CLINT1 | NUDT21 |
| CLIP1 | TWF1 |
| CLIP3 | PNMAL1 |
| CLIP4 | OSMR |
| CLIP4 | RND3 |
| CLK2 | ARNT |
| CLK2 | PTCD3 |
| CLPP | CSNK1G2 |
| CLPP | KHSRP |
| CLPP | POLR2E |
| CLPTM1L | CLPTM1L |
| CMAS | DNM1L |
| CMAS | MAPRE1 |
| CMAS | TBL1XR1 |
| CMPK1 | GPBP1L1 |
| CMTM4 | ELMO3 |
| CNBP | IL20RB |
| CNBP | MAPK1 |
| CNBP | MSL2 |
| CNBP | PSMD12 |
| CNBP | TSN |
| CNBP | UGP2 |
| CNIH2 | CNIH2 |
| CNIH4 | FH |
| CNOT1 | MON1B |
| CNOT3 | FIZ1 |
| CNOT3 | IRF3 |
| CNOT3 | PNKP |
| CNOT3 | PPP2R1A |
| CNOT3 | TPIM28 |
| CNOT3 | ZNF574 |
| CNOT4 | LUC7L2 |
| CNOT4 | ZNF212 |
| CNOT8 | SKIV2L2 |
| CNTN4 | CNTN4 |
| COBRA1 | GTF3C5 |
| COG2 | ZNF672 |
| COIL | MED1 |
| COMMD10 | APPL2 |
| COMMD10 | GIN1 |
| COMMD10 | MAPK9 |
| COMMD10 | MATR3 |
| COMMD10 | RARS |
| COPB2 | B4GALT4 |
| COPB2 | GFPT1 |
| COPB2 | SENP2 |
| COPS5 | ARPC5 |
| COPS5 | ATP6V1C1 |
| COPS5 | HRSP12 |
| COPS5 | IMPAD1 |
| COPS5 | MAPRE1 |
| COPS5 | POLR2K |
| COPS8 | DGUOK |
| COPS8 | LANCL1 |
| COPS8 | MYEOV2 |
| COPS8 | PRKD3 |
| COPS8 | RNF25 |
| COQ4 | COQ4 |
| CORO1B | RAB1B |
| COX4I1 | TRAPPC2L |
| COX4NB | COX4NB |
| COX5A | MRPL46 |
| COX6C | UQCRB |
| CPA5 | CPA5 |
| CPA6 | CPA6 |
| CPN2 | TACR1 |
| CPNE1 | ADNP |
| CPNE1 | HSP90AB1 |
| CPNE1 | RANBP2 |
| CPSF7 | ADRBK1 |
| CPSF7 | DDB1 |
| CPSF7 | MEN1 |
| CPSF7 | NAA40 |
| CPSF7 | PRPF19 |
| CPSF7 | RBM14 |
| CPSF7 | SF3B2 |
| CRAMP1L | USP7 |
| CRBN | TOP2B |
| CRBN | WDR48 |
| CREB1 | GTF3C3 |
| CREB3L2 | CALU |
| CREBZF | SPCS2 |
| CRLS1 | ITPA |
| CROCC | UBR4 |
| CSGALNAC | CSGALNACT1 |
| CSH1 | KCNH6 |
| CSH1 | SGCA |
| CSH1 | ST8SIA3 |
| CSH2 | SLAMF1 |
| CSH2 | TACR1 |
| CSHL1 | CD84 |
| CSHL1 | CHRNA4 |
| CSHL1 | EPHB1 |
| CSHL1 | FCGR3A |
| CSHL1 | FCGR3B |
| CSHL1 | LY9 |
| CSHL1 | MAPK4 |
| CSHL1 | SLAMF1 |
| CSNK1G2 | MBD3 |
| CSNK1G2 | PIAS4 |
| CSNK1G2 | PIP5K1C |
| CSNK1G2 | POLRMT |
| CSNK1G2 | RNF126 |
| CSNK1G2 | SLC39A3 |
| CSNK1G2 | TYK2 |
| CSNK1G3 | GIN1 |
| CSNK2A1 | ZCCHC3 |
| CSPP1 | RBM128 |
| CST3 | CD63 |
| CST6 | CAPN1 |
| CST6 | CST6 |
| CST6 | RHOD |
| CSTA | CSTA |
| CTBP2 | TCF7L2 |
| CTNNA1 | GNS |
| CTNNA1 | MGAT4B |
| CTNNBL1 | DHX35 |
| CTPS | CDC7 |
| CTR9 | PSMA1 |
| CT5A | GNS |
| CTSW | CTSW |
| CTTN | CCND1 |
| CTTN | CD59 |
| CTTN | LRP10 |
| CTTN | PPFIA1 |
| CTTN | PRSS23 |
| CTTN | TWF1 |
| CTU2 | COX4NB |
| CUEDC1 | CUEDC1 |
| CUL3 | NCL |
| CUL9 | EHMT2 |
| CWC27 | CWC27 |
| CWC27 | TAF9 |
| CWF19L2 | ZNF202 |
| CXCL13 | DMP1 |
| CXorf40B | IDH3G |
| CXorf65 | CXorf65 |
| CYB561 | EFNA1 |
| CYB561 | FOXA1 |
| CYB561D1 | CYB561D1 |
| CYB5R1 | ADIPOR1 |
| CYB5R4 | TAB2 |
| CYBA5C3 | TMEM138 |
| CYC1 | MRPL13 |
| CYC5 | SNX13 |
| CYP3A5 | CYP3A5 |
| DAB2 | CD63 |
| DAD1 | TMED10 |
| DAP3 | MRPL9 |
| DARS2 | HRSP12 |
| DARS2 | MRPL13 |
| DARS2 | NDUFB5 |
| DARS2 | SENP2 |
| DAXX | E2F3 |
| DAZAP1 | KDM4B |
| DAZAP1 | NCLN |
| DAZAP1 | RNF126 |
| DBF4 | EZH2 |
| DBF4 | POP7 |
| DBF4 | SLC25A40 |
| DBNL | YKT6 |
| DCAF11 | L2HGDH |
| DCAF11 | RBM23 |
| DCAF15 | ATP13A1 |
| DCAF15 | E2F1 |
| DCAF15 | FARSA |
| DCAF15 | ILF3 |
| DCAF15 | RAVER1 |
| DCAF15 | UPF1 |
| DCAF15 | ZNF787 |
| DCAF6 | DCAF6 |
| DCAF7 | DCAF7 |
| DCK | ELF2 |
| DCLRE1B | CDCA8 |
| DCLRE1C | DCLRE1C |
| DCLRE1C | MLLT10 |
| DCLRE1C | ZNF33A |
| DCTN4 | RIOK2 |
| DCTN4 | YAF2 |
| DCTPP1 | TMEM186 |
| DCUN1D2 | CUL4A |
| DDB1 | MEN1 |
| DDHD1 | DDHD1 |
| DDRGK1 | CENPB |
| DDX1 | PSMD12 |
| DDX10 | ACAT1 |
| DDX10 | BUD13 |
| DDX11 | RBL1 |
| DDX18 | HSPD1 |
| DDX18 | SSB |
| DDX18 | XRCC5 |
| DDX21 | MRPS16 |
| DDX23 | TROAP |
| DDX28 | USP10 |
| DDX28 | ZNF276 |
| DDX41 | TCOF1 |
| DDX42 | BPTF |
| DDX42 | DCAF7 |
| DDX47 | YARS2 |
| DDX49 | UPF1 |
| DDX50 | HNRNPH3 |
| DDX50 | NSMCE4A |
| DDX51 | PUS1 |
| DDX54 | OGFOD2 |
| DDX55 | RFC45 |
| DECR2 | DECR2 |
| DEDD | ARNT |
| DEDD | USP21 |
| DEFB118 | CACNG6 |
| DEFB118 | GLP1R |
| DEFB118 | HOXB1 |
| DEGS1 | ARPC5 |
| DEK | SMC4 |
| DENND1C | VAV1 |
| DENND4B | E2F3 |
| DEPDC1 | RAD54L |
| DERL1 | RNF139 |
| DERL1 | SENP2 |
| DGCR14 | ZC3H7B |
| DHP5 | CDKN2D |
| DHX29 | MOCS2 |
| DHX29 | NDUFS4 |
| DHX29 | TAF9 |
| DHX34 | EXOSC5 |
| DHX34 | STRN4 |
| DHX34 | ZNF574 |
| DHX35 | DHX35 |
| DIABLO | DIABLO |
| DIDO1 | ADNP |
| DIRC2 | GPRC5A |
| DIS3L | PARP16 |
| DLAT | BUD13 |
| DLD | LUC7L2 |
| DLD | SLMO2 |
| DLG1 | DAZAP2 |
| DLG1 | UBXN4 |
| DLG5 | BAG3 |
| DLX4 | DLX4 |
| DMKN | PPP1R13L |
| DNA2 | MKI67 |
| DNAJB11 | DNAJB11 |
| DNAJB4 | CYR61 |
| DNAJB6 | CALU |
| DNAJB8 | DNAJB8 |
| DNAJC21 | RAD1 |
| DNAJC30 | FASTK |
| DNAJC8 | DNAJC8 |
| DNASE1L2 | E4F1 |
| DNASE1L2 | LUC7L |
| DNASE2 | DNASE2 |
| DNM1L | TBL1XR1 |
| DNMT1 | GTPBP3 |
| DNMT1 | RANBP3 |
| DOCK5 | ASPH |
| DOLPP1 | GTF3C5 |
| DPAGT1 | SLC37A4 |
| DPH2 | PPIH |
| DPM1 | MOCS3 |
| DPY19L4 | ATP6V1C1 |
| DPY19L4 | PTK2 |
| DPYS | DPYS |
| DPYSL2 | PNMA2 |
| DRAP1 | CD59 |
| DRG1 | L3MBTL2 |
| DRG1 | SF3A1 |
| DSCC1 | BIRC5 |
| DSCC1 | DSCC1 |
| DSCC1 | MCM3 |
| DSCC1 | PCNA |
| DSCC1 | TRA2B |
| DSN1 | TIMELESS |
| DSP | F11R |
| DSTN | ARPC1A |
| DSTN | ASPH |
| DSTN | KDELR2 |
| DSTN | PTK2 |
| DSTN | RHEB |
| DSTYK | DSTYK |
| DTL | CCNE2 |
| DTL | HNRNPU |
| DTL | RFC4 |
| DTL | TOPBP1 |
| DTL | ZNF672 |
| DTNBP1 | NUP153 |
| DUS1L | ICT1 |
| DUS3L | HNRNPM |
| DUS3L | RNF126 |
| DUS4L | DUS4L |
| DUSP14 | PTRF |
| DYM | BCL2 |
| DYM | TXNL1 |
| E2F1 | H1FX |
| E2F1 | MCM7 |
| E2F1 | TUBA1B |
| E2F1 | UBE2C |
| E2F2 | CDC7 |
| E4F1 | DNASE1L2 |
| E4F1 | E4F1 |
| E4F1 | MAZ |
| E4F1 | SOLH |
| E4F1 | USP7 |
| E4F1 | ZNF500 |
| EAF1 | TOP2B |
| EAF1 | WDR48 |
| EAPP | FBXO34 |
| EBF1 | EBF1 |
| ECD | GLRX3 |
| ECHDC3 | ECHDC3 |
| ECSIT | WDR83 |
| ECT2 | RACGAP1 |
| EDC4 | KARS |
| EDC4 | TERF2 |
| EDC4 | ZNF335 |
| EEF1D | PYCRL |
| EEF1E1 | NMD3 |
| EFEMP1 | CRIM1 |
| EFEMP1 | OSMR |
| EFNA1 | CGN |
| EFNB2 | KLF5 |
| EFTUD2 | AATF |
| EGFR | CTTN |
| EGFR | OSMR |
| EHBP1 | EHBP1 |
| EHBP1L1 | CAPN1 |
| EHD1 | CAPN1 |
| EHF | RHOD |
| EHMT1 | GTF3C5 |
| EHMT2 | LY6G5B |
| EHMT2 | TRIM27 |
| EIF2B1 | GPN3 |
| EIF2C2 | CYC1 |
| EIF2C2 | RAD21 |
| EIF2S3 | MBTPS2 |
| EIF3B | DDX56 |
| EIF3B | HEATR2 |
| EIF3B | TBRG4 |
| EIF3H | UBR5 |
| EIF3K | EXOSC5 |
| EIF3K | RPS11 |
| EIF5 | ZFYVE21 |
| ELAVL1 | HNRNPM |
| ELF3 | C1orf106 |
| ELF5 | ELF5 |
| ELL | AKAP8 |
| ELOF1 | ASNA1 |
| ELOF1 | FARSA |
| ELOVL1 | PLEC |
| ELOVL4 | ELOVL4 |
| ELP2 | ELP2 |
| ELP3 | BIN3 |
| ELP3 | CNOT7 |
| ELP3 | TRIM35 |
| EMD | IDH3G |
| EML3 | MAP3K11 |
| EMP1 | FHL2 |
| EMP1 | HEBP1 |
| EMP1 | RAB11FIP5 |
| EMP1 | TNFRSF1A |
| ENDOG | ENDOG |
| ENO1 | TMED5 |
| ENO2 | STX2 |
| ENOPH1 | HNRNPD |
| ENY2 | HRSP12 |
| EPAS1 | LAPTM4A |
| EPHA1 | TINAGL1 |
| EPHB2 | EGFR |
| EPN3 | C1orf115 |
| EPN3 | LAMA5 |
| EPS15L1 | TNPO2 |
| EPS8 | TWF1 |
| EPS8L1 | EPS8L1 |
| ERBB2 | ERBB2 |
| ERBB2 | SLC16A5 |
| ERCC1 | ERCC1 |
| ERCC2 | ERCC2 |
| ERCC8 | SKIV2L2 |
| ERGIC2 | STRAP |
| ERGIC3 | PIGT |
| ERLIN1 | VPS25A |
| ERN1 | ERN1 |
| ESPL1 | RACGAP1 |
| ESPL1 | SENP1 |
| ESR1 | ESR1 |
| ESR1 | GCM2 |
| ESRP1 | KCNK1 |
| ESRP1 | MAL2 |
| ESRP1 | S100A14 |
| ESRP2 | CDH3 |
| ETFB | ETFB |
| EV12A | EV12A |
| EVL | EVL |
| EXO1 | CCNE2 |
| EXO1 | KIF14 |
| EXOC5 | DHRS7 |
| EXOG | RBM5 |
| EXOG | WDR48 |
| EXOSC1 | CWF19L1 |
| EXOSC9 | CENPE |
| EXOSC9 | MAD2L1 |
| EXT1 | EFEMP1 |
| EYA3 | DNAJC8 |
| EZH1 | SYNRG |
| EZH2 | LUC7L2 |
| EZH2 | ZNF212 |
| F11R | C1orf106 |
| F11R | CD2AP |
| F11R | F11R |
| F11R | GRHL2 |
| F3 | EGFR |
| F3 | GBP3 |
| FAF1 | ITGB3BP |
| FAHD1 | FAHD1 |
| FAM105A | FAM105A |
| FAM13B | FAM13B |
| FAM173A | MPG |
| FAM173A | STUB1 |
| FAM193B | CLK4 |
| FAM193B | MAPKSIP3 |
| FAM20C | FAM20C |
| FAM3A | IK8KG |
| FAM58A | NSDHL |
| FAM76B | BUD13 |
| FAM76B | HINFP |
| FAM83H | ANXA9 |
| FAM83H | CGN |
| FAM83H | F11R |
| FAM84B | EVPL |
| FAM91A1 | RNF139 |
| FANCG | VCP |
| FANCI | CCNB2 |
| FANCI | RFC4 |
| FANCL | MSH2 |
| FANCM | L2HGDH |
| FARSA | ATP13A1 |
| FARSA | ILF3 |
| FARSA | PIN1 |
| FARSA | TNPO2 |
| FARSA | UPF1 |
| FASTK | GNB2 |
| FASTKD2 | HSPE1 |
| FASTKD3 | MTRP |
| FASTKD5 | ITPA |
| FBL | MCM2 |
| FBL | PRMT1 |
| FBL | RUVBL2 |
| FBR5 | PRR14 |
| FBR5 | SETD1A |
| FBR5 | ZNF646 |
| FBR5 | ZNF768 |
| FBXL18 | RAC1 |
| FBXL19 | FUS |
| FBXL6 | RECQL4 |
| FBXO18 | ATP5C1 |
| FBXO18 | FBXO18 |
| FBXO18 | KIN |
| FBXO28 | HRSP12 |
| FBXO46 | VRK3 |
| FBXW5 | EDF1 |
| FCAR | HAMP |
| FCAR | KLK2 |
| FCAR | LILRB3 |
| FDX1L | ASNA1 |
| FDXACB1 | HMBS |
| FERMT1 | CLDN4 |
| FERMT1 | KLF5 |
| FERMT2 | ACTN1 |
| FERMT2 | EML1 |
| FETUB | C6 |
| FGD2 | AIF1 |
| FGFBP1 | CDS1 |
| FGFR1OP | FGFR1OP |
| FGFR2 | FGFR2 |
| FH | HRSP12 |
| FHIT | FHIT |
| FHL2 | RALB |
| FIZ1 | FIZ1 |
| FIZ1 | TRIM28 |
| FKBP4 | FKBP4 |
| FKBP5 | FKBP5 |
| FKBP8 | ATP13A1 |
| FKBP8 | PRKCSH |
| FLAD1 | NDUFS2 |
| FLJ23867 | S100A16 |
| FLNA | FLNA |
| FNDC3B | AMOTL2 |
| FNDC3B | IL1R1 |
| FNDC3B | LEPREL1 |
| FNDC3B | OSMR |
| FNDC3B | TNFRSF1A |
| FNTA | GOLGA7 |
| FNTA | THAP1 |
| FNTA | UBE2V2 |
| FNTA | VDAC3 |
| FOSL1 | CD59 |
| FOXA1 | FOXA1 |
| FOXA1 | GPX2 |
| FOXA2 | FOXA2 |
| FOXI1 | FOXI1 |
| FOXJ3 | GPBP1L1 |
| FOXK2 | FOXK2 |
| FOXM1 | E2F1 |
| FOXO3 | ASF1A |
| FOXR1 | FOXR1 |
| FOXRED1 | ACAD8 |
| FOXRED2 | L3MBTL2 |
| FPGS | FPGS |
| FSTL1 | DCBLD2 |
| FSTL1 | FSTL1 |
| FTSID2 | CNPY3 |
| FUBP1 | FUBP1 |
| FUBP1 | PTBP2 |
| FUBP1 | SFPQ |
| FXR2 | RNF167 |
| FXYD3 | EPS8L1 |
| FXYD3 | STX19 |
| FZD6 | ARFGEF1 |
| FZD6 | DLG1 |
| FZR1 | RNF126 |
| G3BP2 | G3BP2 |
| G3BP2 | LARP7 |
| G3BP2 | RCHY1 |
| G6PC3 | G6PC3 |
| GABARAPL | GABARAPL2 |
| GABPB1 | AQR |
| GABPB1 | RFX7 |
| GABRB2 | GABRB2 |
| GADD45GN | NDUFA11 |
| GADD45GN | NDUFB7 |
| GAPVD1 | GAPVD1 |
| GATAD1 | KRIT1 |
| GATAD2B | MDM4 |
| GATC | RFC5 |
| GATC | SNRPF |
| GBP3 | BCAR3 |
| GBP3 | EGFR |
| GCDH | GTPBP3 |
| GCFC1 | HMGN1 |
| GDE1 | ARPC5 |
| GDE1 | IARS2 |
| GDI1 | FAM50A |
| GDI2 | RAB23 |
| GDI2 | SRP9 |
| GEMIN6 | MRPS7 |
| GEMIN7 | BCL2L12 |
| GFER | AMDHD2 |
| GFER | MLST8 |
| GFM2 | TAF9 |
| GFPT2 | OSMR |
| GGA1 | L3MBTL2 |
| GGA1 | TRMT2A |
| GGA3 | TAOK1 |
| GH2 | CRP |
| GIN1 | PRKAA1 |
| GIN1 | YAF2 |
| GINS1 | BUB1 |
| GINS1 | CCNE2 |
| GINS1 | MYBL2 |
| GINS1 | UBE2C |
| GIPC1 | NR2F6 |
| GIT1 | TCAP |
| GIT1 | USP36 |
| GJA1 | GJA1 |
| GJB3 | CLDN1 |
| GLDC | GLDC |
| GLE1 | POLE3 |
| GLE1 | SPTLC1 |
| GLMN | RPAP2 |
| GLP1R | SLC22A7 |
| GLRX3 | ALDH18A1 |
| GLRX3 | CWF19L1 |
| GLRX5 | DDX24 |
| GLRX5 | PAPOLA |
| GLTSCR2 | MZF1 |
| GLTSCR2 | SNRPA |
| GLTSCR2 | VRK3 |
| GLUD1 | PPA1 |
| GMNN | MDC1 |
| GMNN | PARP1 |
| GNA11 | ZNF358 |
| GNAI3 | ILF2 |
| GNAI3 | RWDD3 |
| GNB2L1 | NOP16 |
| GNG12 | F3 |
| GNG12 | LEPROT |
| GNG12 | NOTCH2 |
| GNG2 | GNG2 |
| GNG5 | GNG5 |
| GNL1 | MRPL2 |
| GNL2 | PPIH |
| GNPAT | FH |
| GNPDA1 | MGAT4B |
| GNS | ATP6V1C1 |
| GNS | DAB2 |
| GNS | ITFG1 |
| GNS | SQSTM1 |
| GOLGA7 | ASH2L |
| GOSR1 | GOSR1 |
| GPATCH1 | STRN4 |
| GPBP1L1 | GPBP1L1 |
| GPHN | EXOC5 |
| GPN3 | CCDC59 |
| GPR125 | GPR125 |
| GPR133 | GPR133 |
| GPR15 | GPR15 |
| GPR22 | GPR22 |
| GPR25 | GPR25 |
| GPR68 | GPR68 |
| GPRC5C | ABCC3 |
| GPS1 | MRPS7 |
| GPS2 | PHF23 |
| GPSM3 | AIF1 |
| GPX8 | GLT8D2 |
| GPX8 | NUAK1 |
| GPX8 | PAM |
| GPX8 | SNX24 |
| GPX8 | TGFBI |
| GPX8 | TNFRSF1A |
| GRAMD3 | EGFR |
| GRB7 | ERBB2 |
| GRB7 | GRHL2 |
| GRB7 | ITGB4 |
| GRHL2 | ITGB4 |
| GRHL2 | S100A14 |
| GRHL2 | STX19 |
| GRTP1 | CLDN4 |
| GRTP1 | KLF5 |
| GSPT1 | USP7 |
| GSTK1 | SLC12A9 |
| GTF2F1 | CSNK1G2 |
| GTF2F1 | KHSRP |
| GTF2F1 | POLR2E |
| GTF2H1 | CAPRIN1 |
| GTF2H1 | PSMC3 |
| GTF3C1 | E4F1 |
| GTF3C1 | ZNF500 |
| GTF3C3 | CWC22 |
| GTF3C3 | PMS1 |
| GTF3C3 | RAB1A |
| GTPBP1 | TRMT2A |
| GTPBP3 | GTPBP3 |
| GTPBP3 | ILF3 |
| GTPBP4 | SUV39H2 |
| GTPBP4 | UPF2 |
| GTSE1 | KIAA1524 |
| GUCA1B | GUCA1B |
| GYS2 | GYS2 |
| H2AFV | GTF2I |
| H2AFX | MLL |
| H3F3B | H3F3B |
| H3F3B | TIA1 |
| HAT1 | CCDC138 |
| HAT1 | MSH6 |
| HAT1 | PNO1 |
| HAUS1 | TXNL1 |
| HAUS4 | C14orf93 |
| HAUS5 | LIG1 |
| HAUS5 | MAP4K1 |
| HAUS5 | MCM3 |
| HAUS5 | POLQ |
| HAUS6 | PSIP1 |
| HAUS7 | EMD |
| HAUS8 | MED26 |
| HBP1 | UBE2H |
| HCC5 | MBTPS2 |
| HCFC2 | HCFC2 |
| HDAC2 | HDAC2 |
| HDDC3 | MRPL46 |
| HDGFRP2 | PIN1 |
| HDHD2 | TXNL1 |
| HEBP1 | HEBP1 |
| HEG1 | OSMR |
| HEXB | DAB2 |
| HEXB | IL6ST |
| HEXB | MGAT4B |
| HEXDC | MBTD1 |
| HGS | GGA3 |
| HGS | SLC38A10 |
| HHLA2 | HAMP |
| HINFP | BUD13 |
| HIPK1 | HIPK1 |
| HIPK2 | HIPK2 |
| HIST1H2AE | HIST1H2AE |
| HIST1H2AK | HIST1H2AE |
| HIST1H2AM | HIST1H2AE |
| HIST1H2BD | HIST1H1C |
| HIST1H2BE | HIST1H1C |
| HIST1H2BE | HIST1H3E |
| HIST1H2BF | HIST1H1C |
| HIST1H2BF | HIST1H3E |
| HIST1H2BG | HIST1H1C |
| HIST1H2BH | HIST1H1C |
| HIST1H2BI | HIST1H1C |
| HIST1H3B | HIST1H4F |
| HIST1H3D | HIST1H3E |
| HIST1H4A | HIST1H2AJ |
| HIST1H4A | HIST1H3E |
| HIST1H4A | HIST1H3I |
| HIST1H4A | HIST1H3J |
| HIST1H4A | HIST1H4A |
| HIST1H4A | HIST1H4B |
| HIST1H4A | HIST1H4D |
| HIST1H4A | HIST1H4F |
| HIST1H4A | HIST1H4I |
| HIST1H4A | HIST1H4L |
| HIST1H4E | HIST1H4E |
| HIST1H4E | HIST1H4F |
| HIST1H4H | HIST1H4C |
| HIST1H4H | HIST1H4E |
| HLA-DOA | SLC22A7 |
| HLA-E | HLA-E |
| HLA-E | TAP2 |
| HLA-G | HLA-F |
| HLX | HLX |
| HMBS | SLC37A4 |
| HMCN1 | HMCN1 |
| HMGB1 | CTCF |
| HMGB1 | GTF3A |
| HMGB1 | MYBL2 |
| HMGN4 | HMGN4 |
| HMMR | CDC25C |
| HMMR | HMMR |
| HNF1B | HNF1B |
| HNF4A | HNF4A |
| HNF4A | TSPO2 |
| HNRNPA0 | LMNB1 |
| HNRNPA2B1 | TPX2 |
| HNRNPC | C14orf166 |
| HNRNPC | EXOC5 |
| HNRNPD | CENPE |
| HNRNPD | HNRNPD |
| HNRNPF | GDI2 |
| HNRNPH3 | KIF11 |
| HNRNPM | AKAP8 |
| HNRNPM | CHAF1A |
| HNRNPM | NUP62 |
| HNRNPM | POLD1 |
| HNRNPUL1 | GRWD1 |
| HNRNPUL1 | SAE1 |
| HNRNPUL1 | SPHK2 |
| HNRNPUL1 | TACR1 |
| HNRNPUL1 | ZNF611 |
| HNRPDL | HNRNPD |
| HOMER2 | HOMER2 |
| HOXA10 | HOXA9 |
| HOXA13 | HOXA13 |
| HOXB1 | GH1 |
| HOXB7 | HOXB5 |
| HOXC10 | HOXC9 |
| HOXC6 | HOXC8 |
| HOXC9 | HOXC8 |
| HPX | HPX |
| HRH3 | FOXN4 |
| HRSP12 | C20orf30 |
| HRSP12 | SRP9 |
| HS6ST3 | HS6ST3 |
| HSF1 | RECQL4 |
| HSH2D | GMIP |
| HSP90AB1 | SLC29A1 |
| HSPA14 | NUDT5 |
| HSPA18 | HSPA1B |
| HSPA4 | RAD50 |
| HSPA4 | TTC37 |
| HSPA4 | YAF2 |
| HSPBP1 | TRIM28 |
| HTATIP2 | HTATIP2 |
| HTR7P1 | HEBP1 |
| HUS1 | YKT6 |
| IARS2 | FH |
| IARS2 | KLHL12 |
| IARS2 | MRPL13 |
| IDH3G | IDH3G |
| IER3IP1 | TXNL1 |
| IGFBP3 | EGFR |
| IGFBP3 | OSMR |
| IGFBP6 | C1R |
| IGSF9 | MAL2 |
| IKZF3 | IKZF3 |
| IKZF5 | NSMCE4A |
| IL10RA | IL10RA |
| IL13RA1 | PLS3 |
| IL2RG | CXorf65 |
| IL3 | IL12B |
| IL3 | SIGLEC8 |
| IL31RA | IL31RA |
| ILF2 | ARFGEF1 |
| ILF2 | BRIX1 |
| ILF2 | CCT5 |
| ILF2 | HRSP12 |
| ILF2 | MRPL13 |
| ILF2 | POLR3C |
| ILF2 | RCOR3 |
| ILF2 | TAF1A |
| ILF3 | FARSA |
| ILF3 | GTPBP3 |
| ILF3 | RAVER1 |
| ILF3 | SNRPA |
| IMMP1L | IMMP1L |
| IMPA2 | IMPA2 |
| INADL | TACSTD2 |
| ING1 | TFDP1 |
| ING3 | LUC7L2 |
| INHBA | OSMR |
| INO80E | PRR14 |
| INSM1 | INSM1 |
| INTS1 | BRD9 |
| INTS10 | HMBOX1 |
| INTS12 | INTS12 |
| INTS12 | USO1 |
| INTS2 | COIL |
| INTS5 | MAPSK11 |
| INTS5 | SF1 |
| IQCE | RAC1 |
| IRAK1 | IDH3G |
| IRAK1 | IKBKG |
| IREB2 | IREB2 |
| IREB2 | RFX7 |
| IREB2 | SLTM |
| IRF2BP1 | SPHK2 |
| IRF6 | SOX13 |
| IRF9 | PSME1 |
| IRX3 | IRX5 |
| ISCA1 | SPTLC1 |
| ISG20L2 | MRPL9 |
| ISLR | ISLR |
| ITCH | DNAJB6 |
| ITCH | TBL1XR1 |
| ITCH | UBE2H |
| ITFG1 | MBTPS1 |
| ITGA3 | PTRF |
| ITGAL | TPSAB1 |
| ITGB3BP | CDC7 |
| ITGB3BP | PRPF38A |
| ITGB5 | NCEH1 |
| ITPR1 | ITPR1 |
| ITPR3 | ITPR3 |
| JAGN1 | THUMPD3 |
| JTB | MRPL9 |
| JUN | JUN |
| JUP | GRB7 |
| JUP | JUP |
| KARS | NAE1 |
| KBTBD6 | KBTBD7 |
| KCNH2 | KCNH2 |
| KCNJ5 | SLC22A6 |
| KCNK3 | KCNK3 |
| KCNMB2 | KCNMB2 |
| KCTD13 | AXIN1 |
| KCTD13 | ZNF668 |
| KCTD2 | RECQL5 |
| KCTD20 | RAB23 |
| KDELC2 | KDELC2 |
| KDELR2 | CALU |
| KDELR2 | OSMR |
| KDM1B | KDM1B |
| KDM2A | CDK2AP2 |
| KDM2A | PTPRCAP |
| KDM5C | KDM5C |
| KDM6B | WRAP53 |
| KHDR852 | MYH7 |
| KHSRP | CSNK1G2 |
| KHSRP | HNRNPM |
| KHSHP | ILF3 |
| KIAA0182 | KIAA0182 |
| KIAA0195 | RECQL5 |
| KIAA0664 | MINK1 |
| KIAA0664 | RNF167 |
| KIAA0664 | USP36 |
| KIAA1279 | MARCH5 |
| KIAA1429 | KIAA1429 |
| KIAA1522 | EGFR |
| KIAA1522 | INADL |
| KIAA1522 | RHBDL2 |
| KIAA1522 | SLC2A1 |
| KIAA1522 | TINAGL1 |
| KIAA1967 | CNOT7 |
| KIAA2026 | AK3 |
| KIAA2026 | PSIP1 |
| KIF12 | KIF12 |
| KIF1B | RNF11 |
| KIF1B | SKI |
| KIF1C | MINK1 |
| KIF20A | CDC25C |
| KIF20A | HMMR |
| KIF2A | CWC27 |
| KIF2C | CKS1B |
| KIF2C | FAF1 |
| KIF2C | KHDRBS1 |
| KIF2C | PPIH |
| KIFC1 | E2F3 |
| KIFC1 | TUBB |
| KIR2DL3 | KIR2DL1 |
| KIR2DL3 | KIR2DL4 |
| KLC3 | KLK5 |
| KLF5 | AHR |
| KLF5 | ID1 |
| KLHL9 | KLHL9 |
| KLK10 | KLK11 |
| KLK10 | KLK7 |
| KLK10 | KLK8 |
| KLK10 | KLK9 |
| KLK11 | KLK10 |
| KIK14 | KLK14 |
| KLK5 | KLK6 |
| KLK6 | EPS8L1 |
| KLK6 | KLK6 |
| KLK6 | KLK7 |
| KLK8 | KLK9 |
| KNTC1 | ESPL1 |
| KNTC1 | NFYB |
| KNTC1 | SBNO1 |
| KPNA5 | ASF1A |
| KPTN | STRN4 |
| KRAS | KRAS |
| KRI1 | AKAP8L |
| KRI1 | C19orf43 |
| KRI1 | HNRNPM |
| KRIT1 | PEX1 |
| KRIT1 | ZKSCAN5 |
| KRT19 | ITGB4 |
| KRT19 | JUP |
| KRT32 | KRT32 |
| L2HGDH | L2HGDH |
| L3MBTL2 | ACO2 |
| L3MBTL2 | L3MBTL2 |
| L3MBTL2 | TRMT2A |
| LAMA5 | SLPI |
| LAMB1 | CALU |
| LAMC1 | ASPH |
| LAMC1 | NOTCH2 |
| LAMC2 | EPCAM |
| LAMC2 | F11R |
| LAPTM4A | ASAP2 |
| LARP4B | FBXO18 |
| LARP4B | MLLT10 |
| LARP7 | C4orf21 |
| LARP7 | CCNG2 |
| LARP7 | HMGN1 |
| LARP7 | INTS12 |
| LARP7 | NUP54 |
| LARP7 | RAB28 |
| LASP1 | ABCC3 |
| LATS1 | NUP43 |
| LCP2 | PKD2L2 |
| LEMD3 | ZBTB39 |
| LENEP | AIF1 |
| LENG9 | LENG9 |
| LEO1 | AQR |
| LEPREL1 | PPP2R3A |
| LEPROT | EGFR |
| LEFROT | NOTCH2 |
| LEPROT | PIGK |
| LEPROTL1 | ATP6V1B2 |
| LGALS3BP | ABCC3 |
| LHX4 | LHX4 |
| LILRA1 | LILRB1 |
| LILRA2 | KIR2DL1 |
| LILRA2 | KLK2 |
| LILRA2 | LILRB1 |
| LIMD2 | MAP3K3 |
| LIME1 | CPSF1 |
| LIN37 | POLR2I |
| LIN37 | U2AF1L4 |
| LIPH | FXYD3 |
| LLGL2 | EPCAM |
| LLPH | CCT2 |
| LMBRD1 | LMBRD1 |
| LMO2 | LMO2 |
| LOC100128822 | MLL3 |
| LOC400657 | BCL2 |
| LOC81691 | ERI2 |
| LOC81691 | KIF14 |
| LOC81691 | NEK2 |
| LONP2 | LONP2 |
| LOXL2 | MYBL1 |
| LPP | AMOTL2 |
| LPP | CD63 |
| LPP | EMP1 |
| LPP | OSMR |
| LPP | WWTR1 |
| LRIG2 | HIPK1 |
| LRP10 | SERPINB6 |
| LRP12 | AKT3 |
| LRRC16A | DDR1 |
| LRRC37A3 | SMARCE1 |
| LSG1 | MRPL47 |
| LSM14A | MSL2 |
| LSM14A | ZNF146 |
| LSM3 | CAPN7 |
| LSM3 | CNOT10 |
| LSM3 | MRPS25 |
| LSM3 | THUMPD3 |
| LSM7 | RNF126 |
| LSMD1 | WRAP53 |
| LSR | GPRC5A |
| LSR | STX19 |
| LTB | LTB |
| LTBR | ANXA4 |
| LTBR | GPRC5A |
| LTBR | HEBP1 |
| LUC7L2 | CBLL1 |
| LUC7L2 | CNOT4 |
| LUC7L2 | LUC7L2 |
| LUC7L2 | ZNF212 |
| LUC7L3 | DCAF7 |
| LY6H | LY6H |
| LY6K | OSMR |
| LY85 | AIF1 |
| LYL1 | LYL1 |
| LYPLA2 | LYPLA2 |
| LYRM2 | LYRM2 |
| LZTR1 | TRMT2A |
| MACC1 | AGR2 |
| MACC1 | CDH1 |
| MACC1 | CLDN4 |
| MAF1 | CP5F1 |
| MAG | LEP |
| MAGOH | PPIH |
| MAK16 | UBXN8 |
| MAL2 | ANXA9 |
| MAL2 | ELF3 |
| MAL2 | KCNK1 |
| MAL2 | LAD1 |
| MAMLD1 | FLNA |
| MAN2B1 | ATP13A1 |
| MANBAL | RIN2 |
| MAP1S | ATP13A1 |
| MAP1S | PGLS |
| MAP1S | RAVER1 |
| MAP2K4 | GLOD4 |
| MAP2K4 | PRPSAP2 |
| MAP3K11 | FAM89B |
| MAP3K11 | PITPNM1 |
| MAP3K6 | MAP3K6 |
| MAP4K5 | MNAT1 |
| MAPK1 | UFD1L |
| MAPK14 | ABT1 |
| MAPK8IP3 | USP7 |
| MAPK9 | CANX |
| MAPKAPK5 | MAPKAPK5 |
| MAPRE1 | CCT5 |
| MAPRE1 | CPNE1 |
| MAPRE1 | DNAJB6 |
| MAPRE1 | RPS6KB1 |
| MAPT | MAPT |
| MARCH5 | ERLIN1 |
| MARS2 | BCS1L |
| MATR3 | HNRNPH1 |
| MATR3 | PPWD1 |
| MATR3 | RIOK2 |
| MAZ | MAZ |
| MAZ | MLST8 |
| MBD1 | HDHD2 |
| MBD2 | MBD2 |
| MBD3 | CDC34 |
| MBD3 | DNM2 |
| MBD3 | MLLT1 |
| MBD3 | NCLN |
| MBD3 | PIAS4 |
| MBD3 | PIP5K1C |
| MBD3 | POLD1 |
| MBD3 | POLR2E |
| MBD3 | RNF126 |
| MBD3 | SLC39A3 |
| MBD3 | USF2 |
| MBD4 | SNX4 |
| MBTD1 | POU2F1 |
| MBTD1 | PPM1D |
| MBTD1 | ZNF397 |
| MBTPS1 | DNAJA2 |
| MCM10 | GMNN |
| MCM10 | KIF11 |
| MCM10 | MCM3 |
| MCM10 | TRA2B |
| MCM2 | RAD54L |
| MCM5 | L3MBTL2 |
| MCM5 | TRMT2A |
| MCM7 | CASP2 |
| MCM7 | LUC7L2 |
| MCM8 | MCM8 |
| MCPH1 | CNOT7 |
| MCPH1 | HMBOX1 |
| MCPH1 | WRN |
| MCRS1 | TROAP |
| MDC1 | ABCF1 |
| MDC1 | PARP1 |
| MDH1 | HSPD1 |
| MDM2 | MDM2 |
| MDM4 | PDE7A |
| MDM4 | RAB3GAP2 |
| MDM4 | TOMM20 |
| ME2 | HDHD2 |
| ME2 | TXNL1 |
| MEAF6 | SNIP1 |
| MED1 | DDX42 |
| MED1 | POU2F1 |
| MED13 | DCAF7 |
| MED15 | TRMT2A |
| MED16 | CDC34 |
| MED16 | KDM4B |
| MED16 | NCLN |
| MED16 | PIP5K1C |
| MED16 | POLRMT |
| MED16 | UPF1 |
| MED17 | TMEM126B |
| MED18 | TAF12 |
| MED21 | ATP6V1C1 |
| MED21 | CMAS |
| MED21 | TBL1XR1 |
| MED24 | MED24 |
| MED26 | GTPBP3 |
| MED25 | ILF3 |
| MED25 | RAB8A |
| MED25 | RAVER1 |
| MED25 | TNPO2 |
| MED4 | RB1 |
| MED6 | C14orf166 |
| MED6 | PAPOLA |
| MED7 | HSPA4 |
| MED7 | RNF14 |
| MEGF6 | MEGF6 |
| MELK | VCP |
| MEN1 | MEN1 |
| MEN1 | UBXN1 |
| MET | GPRC5A |
| MET | PRKAG2 |
| MET | UBE2H |
| METTL3 | FANCM |
| METTL6 | DYNC1LI1 |
| MFN1 | DCUN1D1 |
| MFN1 | DNAJC10 |
| MFN1 | ITCH |
| MFN1 | SENP2 |
| MFN1 | TFG |
| MFSD5 | SQSTM1 |
| MGAT4B | HEXB |
| MGAT4B | TBC1D9B |
| MGC16275 | TAOK1 |
| MGRN1 | BCKDK |
| MGRN1 | DNASE1L2 |
| MGRN1 | FAM193B |
| MGRN1 | ZNF500 |
| MIB1 | ZHF24 |
| MICB | TAP1 |
| MIER1 | MIER1 |
| MIER2 | CDC34 |
| MIER2 | PIP5K1C |
| MKI67 | KIF20B |
| MKK5 | NAA20 |
| MKLN1 | CNOT4 |
| MKLN1 | LUC7L2 |
| MKNK1 | MKNK1 |
| MKRN2 | DYNC1LI1 |
| MKRN2 | LSM3 |
| MKRN2 | NR2C2 |
| MLH1 | CCDC12 |
| MLH1 | DYNC1LI1 |
| MLL2 | SUDS3 |
| MLL3 | EZH2 |
| MLL3 | ZNF212 |
| MLL5 | KRIT1 |
| MLLT1 | MLLT1 |
| MLYCD | MLYCD |
| MMADHC | RALB |
| MMADHC | UBXN4 |
| MMP13 | MMP13 |
| MMP7 | MMP7 |
| MNAT1 | PAPOLA |
| MNT | MINK1 |
| MOCS3 | OSGEPL1 |
| MOGAT3 | MOGAT3 |
| MON1B | MON1B |
| MORC2 | MORC2 |
| MORF4L2 | PSMD10 |
| MOSPD3 | TAF6 |
| MPG | MPG |
| MPHOSPH8 | MYCBP2 |
| MPHOSPH9 | MPHOSPH9 |
| MPP1 | MPP1 |
| MPP6 | MPP6 |
| MRE11A | CHEK1 |
| MRE11A | ZBTB44 |
| MRM1 | AATF |
| MRPL13 | CCT5 |
| MRPL13 | DSCC1 |
| MRPL13 | IARS2 |
| MRPL13 | MAPRE1 |
| MRPL13 | PRKAA1 |
| MRPL13 | PRKDC |
| MRPL13 | PSMD12 |
| MRPL13 | SDHC |
| MRPL13 | UBE2V2 |
| MRPL15 | COPS5 |
| MRPL18 | FAM54A |
| MRPL18 | FBXO5 |
| MRPL18 | RNF146 |
| MRPL20 | PARK7 |
| MRPL21 | WDR74 |
| MRPL22 | MRPL22 |
| MRPL3 | DNM1L |
| MRPL3 | PSMD12 |
| MRPL34 | ATP13A1 |
| MRPL34 | GTPBP3 |
| MRPL4 | ATP13A1 |
| MRPL4 | FARSA |
| MRPL4 | GTPBP3 |
| MRPL4 | MRPS12 |
| MRPL4 | RAVER1 |
| MRPL42 | STRAP |
| MRPL46 | MRPL46 |
| MRPL47 | MRPL47 |
| MRPL54 | RNF126 |
| MRPS14 | MRPS14 |
| MRPS17 | DDX56 |
| MRPS17 | POP7 |
| MRPS17 | PSMG3 |
| MRPS17 | UBE2H |
| MRPS18C | HNRNPD |
| MRPS2 | GTF3C4 |
| MRPS25 | CCDC12 |
| MRPS25 | RPL15 |
| MRPS26 | ITPA |
| MRPS26 | NXT1 |
| MRPS28 | MAPRE1 |
| MRPS31 | FAM48A |
| MRPS31 | MED4 |
| MRPS31 | SLC25A15 |
| MRPS33 | FIS1 |
| MRPS34 | CCNF |
| MRPS34 | E4F1 |
| MRPS36 | TAF9 |
| MRPS7 | NME2 |
| MRPS7 | TACO1 |
| MRPS7 | TK1 |
| MRS2 | MRS2 |
| MS4A5 | MS4A5 |
| MSH2 | ACP1 |
| MSH2 | CREB1 |
| MSH2 | FANCL |
| MSH2 | RPIA |
| MSL2 | TBLIXR1 |
| MT2A | ABLIM3 |
| MTA1 | MARK3 |
| MTA2 | SF1 |
| MTBP | DSCC1 |
| MTF2 | LRRC40 |
| MTF2 | PTBP2 |
| MTF2 | RAD54L |
| MTF2 | RBMXL1 |
| MTF2 | RPA2 |
| MTFR1 | C1orf27 |
| MTFR1 | CD46 |
| MTFR1 | ITCH |
| MTFR1 | POLR2K |
| MTFR1 | YWHAZ |
| MTIF2 | GEMIN6 |
| MTIF2 | PNO1 |
| MTIF3 | MTIF3 |
| MTMR14 | ARPC4 |
| MTMR4 | DCAF7 |
| MTMR9 | HMBOX1 |
| MTNR1B | MTNR1B |
| MTPAP | NSUN6 |
| MTX2 | PRKRA |
| MUC20 | PLEKHG6 |
| MXRA5 | MXRA5 |
| MXRA7 | MRC2 |
| MXRA7 | RAB34 |
| MYBL1 | C8orf46 |
| MYBL2 | BUB1 |
| MYBL2 | MCM7 |
| MYBL2 | TOP2A |
| MYBL2 | UBE2C |
| MYC | MYC |
| MYH14 | KLK10 |
| MYH2 | ESR1 |
| MYLK | DCBLD2 |
| MYO1B | RND3 |
| MYO1C | ACADVL |
| MYO1C | KCTD11 |
| MYOT | MYOT |
| MYT1 | KCNH2 |
| MZF1 | LENG8 |
| MZF1 | STRN4 |
| N4BP2L2 | MTMR6 |
| N4BP2L2 | PDS5B |
| NAA10 | NSDHL |
| NAA15 | MAD2L1 |
| NAA15 | NUP54 |
| NAA16 | GTF3A |
| NAA38 | LUC7L2 |
| NAA38 | POT1 |
| NAA50 | PSMD12 |
| NAA50 | RAB1A |
| NACA | NAP1L1 |
| NAE1 | DNAJA2 |
| NAE1 | NAE1 |
| NAE1 | NUDT21 |
| NAGLU | G6PC3 |
| NARG2 | CEP152 |
| NARS2 | DLAT |
| NARS2 | RPS3 |
| NCAPD2 | POLQ |
| NCAPD2 | RACGAP1 |
| NCAPD3 | ACAD8 |
| NCAPD3 | PPP2R1B |
| NCAPH | AURKA |
| NCAPH | BARD1 |
| NCAPH | R3HDM1 |
| NCAPH | RRM2 |
| NCAPH | TPX2 |
| NCAPH2 | GTPBP1 |
| NCBP2 | MRPL3 |
| NCBP2 | PIK3CA |
| NCEH1 | IGFBP6 |
| NCEH1 | ITGA3 |
| NCEH1 | LPP |
| NCK1 | TBL1XR1 |
| NCOA2 | NCOA2 |
| NCOR2 | NCOR2 |
| NCOR2 | SMARCC2 |
| NCR1 | KIR2DL1 |
| NDC80 | CENPA |
| NDEL1 | ZBTB4 |
| NDST1 | GFX8 |
| NDUFA5 | KRIT1 |
| NDUFA5 | LUC7L2 |
| NDUFA8 | ENDOG |
| NDUFAF4 | HSP90AB1 |
| NDUFAF4 | LYRM2 |
| NDUFB2 | NDUFB2 |
| NDUFB5 | MRPL47 |
| NDUFB5 | TBL1XR1 |
| NDUFB5 | UGP2 |
| NDUFB7 | FARSA |
| NDUFB9 | C8orf33 |
| NDUFB9 | DSCC1 |
| NDUFB9 | RNF139 |
| NDUFS2 | NDUFS2 |
| NDUFS7 | RNF126 |
| NDUFS8 | RAB1B |
| NDUFV1 | WDR74 |
| NEIL3 | CENPE |
| NEIL3 | SAP30 |
| NEK1 | NEK1 |
| NEK2 | ANP32E |
| NEK2 | CKS1B |
| NEK7 | ARPC5 |
| NEU3 | NEU3 |
| NEUROG1 | IL4 |
| NEUROG1 | LILRB2 |
| NEUROG1 | NCR1 |
| NFATC2IP | PRR14 |
| NFE2L2 | DNAJC10 |
| NFE2L2 | PNO1 |
| NFKBIL1 | NFKBIL1 |
| NFRKB | CWF19L2 |
| NFS1 | C20orf24 |
| NFX1 | NFX1 |
| NFYB | SCYL2 |
| NFYB | SENP1 |
| NFYB | ZDHHC17 |
| NGB | NGB |
| NGDN | FANCM |
| NGFRAP1 | W8P5 |
| NKIRAS2 | TMUB2 |
| NKRF | PHF6 |
| NLE1 | GART |
| NLE1 | KAT2A |
| NMD3 | GNA13 |
| NMD3 | MRPL3 |
| NMD3 | MSH2 |
| NMD3 | SENP2 |
| NMD3 | TBL1XR1 |
| NMD3 | TFG |
| NMD3 | TOMM22 |
| NMD3 | UGP2 |
| NME1 | MRPL27 |
| NME1 | NME2 |
| NME1 | STRA13 |
| NMNAT3 | NMNAT3 |
| NMT2 | VIM |
| NNMT | PRSS23 |
| NOC2L | MRPL37 |
| NOL11 | BPTF |
| NOL11 | COIL |
| NOL11 | NME1 |
| NOL12 | L3MBTL2 |
| NOL12 | TRMT2A |
| NOL6 | SIGMAR1 |
| NONO | PGK1 |
| NOP2 | DDX54 |
| NOP2 | RR51 |
| NOP58 | EIF5B |
| NOTCH2 | NOTCH2NL |
| NPAT | CHEK1 |
| NPLOC4 | SLC38A10 |
| NPTN | NPTN |
| NPVF | GRM8 |
| NR1I2 | NR1I2 |
| NRBP2 | NRBP2 |
| NRM | TUBB |
| NSL1 | HNRNPU |
| NSL1 | POU2F1 |
| NSL1 | ZNF678 |
| NSMCE2 | RNF139 |
| NSMCE4A | CWF19L1 |
| NSMCE4A | KIF11 |
| NSUN2 | CLPTM1L |
| NSUN2 | MTRR |
| NSUN4 | GPBP1L1 |
| NSUN6 | MLLT10 |
| NTF3 | NTF3 |
| NUBPL | NUBPL |
| NUCB1 | NUCB1 |
| NUDC | DNAJC8 |
| NUDCD1 | DSCC1 |
| NUDCD3 | DDX56 |
| NUDCD3 | KIAA0415 |
| NUDT1 | CDCA8 |
| NUMA1 | SF1 |
| NUP153 | E2F3 |
| NUP153 | PAK1IP1 |
| NUP155 | RAD1 |
| NUP188 | PMPCA |
| NUP205 | H2AFV |
| NUP205 | LUC7L2 |
| NUP205 | ZNF212 |
| NUP205 | ZNF273 |
| NUP54 | CDKN2AIP |
| NUP54 | HNRNPD |
| NUP54 | PAICS |
| NUP54 | POLR2B |
| NUP62 | PRPF31 |
| NUP62 | RUVBL2 |
| NUP85 | NUP85 |
| NUP88 | GSG2 |
| NUSAP1 | BLM |
| NXT1 | NAA20 |
| OAF | OAF |
| OBFC2A | RND3 |
| OCEL1 | YIPF2 |
| OCRL | TCEAL1 |
| OGDH | FBXL18 |
| OGDH | TBRG4 |
| OGDH | ZMIZ2 |
| OIP5 | ARHGAP11A |
| OIP5 | CCNB2 |
| OMP | OMP |
| ORAOV1 | PPFIA1 |
| ORM1 | ORM2 |
| OSBPL11 | IL20RB |
| OSBPL8 | ZDHHC17 |
| OSGEPL1 | B3GNT2 |
| OSGEPL1 | MSH2 |
| OSGEPL1 | PNO1 |
| OSGEPL1 | PRKRA |
| OSMR | IGFBP6 |
| OSMR | IL1R1 |
| OTUD6B | POLR2K |
| OXA1L | RPL36AL |
| OXCT1 | OXCT1 |
| OXNAD1 | HACL1 |
| OXNAD1 | LSM3 |
| P2RX1 | P2RX1 |
| P2RY2 | CAPN1 |
| P2RY2 | SSH3 |
| PA2G4 | TMPO |
| PABPC4 | PABPC4 |
| PAF1 | SAE1 |
| PAF1 | SNRNP70 |
| PAF1 | SYMPK |
| PAFAH1B3 | SAE1 |
| PAK1 | PAK1 |
| PAK1IP1 | NUP153 |
| PALLD | ARSJ |
| PAN2 | ZBTB39 |
| PAN3 | FAM48A |
| PAN3 | MED4 |
| PANK4 | UBE2J2 |
| PAPOLA | C14orf166 |
| PAPOLA | EXOC5 |
| PAPOLA | PAPOLA |
| PARK7 | AURKAIP1 |
| PARL | POLR2H |
| PARP1 | HNRNPU |
| PARP1 | USP21 |
| PARP2 | DLGAP5 |
| PARP8 | PARP8 |
| PARVA | ILK |
| PARVB | PARVB |
| PATZ1 | SREBF2 |
| PAX4 | GHRHR |
| PAX8 | PAX8 |
| PAX9 | PAX9 |
| PAXIP1 | EZH2 |
| PAXIP1 | RSBN1L |
| PBXIP1 | PBXIP1 |
| PCDHA10 | PCDHA2 |
| PCDHA10 | PCDHA4 |
| PCDHA10 | PCDHAC1 |
| PCDHA3 | PCDHAC1 |
| PCDHA3 | PCDHAC2 |
| PCDHA5 | PCDHAC1 |
| PCDHA6 | PCDHA8 |
| PCDHA9 | PCDHA6 |
| PCDHA9 | PCDHAC1 |
| PCDHA9 | PCDHAC2 |
| PCDHAC1 | PCDHA1 |
| PCDHAC1 | PCDHA8 |
| PCDHAC1 | PCDHAC1 |
| PCDHAC2 | PCDHAC1 |
| PCDHB10 | PCDHB2 |
| PCDHB13 | PCDHB2 |
| PCDHB5 | PCDHB2 |
| PCDHB6 | PCDHB2 |
| PCDHGA1 | PCDHGB5 |
| PCDHGA10 | PCDHGB2 |
| PCDHGA10 | PCDHGB3 |
| PCDHGA10 | PCDHGB5 |
| PCDHGA10 | PCDHGC5 |
| PCDHGA9 | PCDHGA1 |
| PCDHGB5 | PCDHGB5 |
| PCDHGB6 | PCDHGA4 |
| PCDHGB7 | PCDHGA8 |
| PCDHGB7 | PCDHGC5 |
| PCDHGC3 | PCDHGA2 |
| PCDHGC3 | PCDHGA3 |
| PCDHGC3 | PCDHGA8 |
| PCDHGC3 | PCDHGB3 |
| PCDHGC3 | PCDHGC5 |
| PCDHGC5 | PCDHGA1 |
| PCDHGC5 | PCDHGA3 |
| PCDHGC5 | PCDHGB2 |
| PCDHGC5 | PCDHGB6 |
| PCID2 | CUL4A |
| PCMT1 | RNF146 |
| PCMTD2 | PAN2 |
| PCSK2 | PCSK2 |
| PCYOX1 | ITGAV |
| PDCD10 | MRPL3 |
| PDCD10 | TFG |
| PDCD10 | UGP2 |
| PDCD2L | PNPT1 |
| PDE12 | ARIH2 |
| PDE48 | PDE4B |
| PDE7A | CLK2 |
| PDE7A | RBM12B |
| PDE8A | TSPAN3 |
| PDE9A | PDE9A |
| PDK1 | PDK1 |
| PDK2 | PDK2 |
| PDP1 | PLAT |
| PDPK1 | DNASE1L2 |
| PDPK1 | E4F1 |
| PDPK1 | USP7 |
| PDPK1 | ZNF500 |
| PDX1 | HNF4A |
| PDX1 | PDX1 |
| PDZD8 | POZD8 |
| PEF1 | PEF1 |
| PEMT | PEMT |
| PERP | DDR1 |
| PERP | DSP |
| PES1 | L3MBTL2 |
| PES1 | POLR1B |
| PES1 | TRMT2A |
| PEX16 | UBXN1 |
| PEX2 | ARPC5 |
| PEX2 | HRSP12 |
| PEX2 | IMPA1 |
| PEX2 | MAPRE1 |
| PEX2 | RNF139 |
| PFDN5 | RPLP0 |
| PGAP3 | ERBB2 |
| PGAP3 | WIPF2 |
| PGGT1B | GDE1 |
| PGGT1B | GIN1 |
| PGGTIB | SNX2 |
| PGLS | SIN3B |
| PGLYRP4 | CDSN |
| PGM3 | RARS2 |
| PGP | E4F1 |
| PHB2 | ITFG2 |
| PHF13 | GNB1 |
| PHF2 | PHF2 |
| PHF20 | PHF20 |
| PHF6 | ZNF280C |
| PHIP | BPTF |
| PHIP | HDAC2 |
| PHIP | KPNA5 |
| PHKA2 | OFD1 |
| PHLDB2 | DCBLD2 |
| PHLDB2 | EFEMP1 |
| PHLDB2 | OSMR |
| PHLDB2 | PRNP |
| PHLPP1 | PHLPP1 |
| PI4K2A | BAG3 |
| PIAS2 | TXNL1 |
| PIAS4 | RNF126 |
| PICALM | RDX |
| PIF1 | CCNB2 |
| PIGK | LEPROT |
| PIGO | SIGMAR1 |
| PIGQ | ZNF500 |
| PIK3CA | RPS6KB1 |
| PIK3CA | TBL1XR1 |
| PIK3R4 | TBL1XR1 |
| PIK3R4 | ZNF148 |
| PIP5K1A | SENP2 |
| PIP5K1A | SLC39A1 |
| PITPNM1 | PTPRCAP |
| PITPNM3 | PITPNM3 |
| PKD1 | E4F1 |
| PKD1 | USP7 |
| PKD2L2 | SLC9A3 |
| PKIA | PKIA |
| PKMYT1 | NFATC2IP |
| PKN2 | PKN2 |
| PKP2 | PARD6B |
| PLAGL2 | DHX35 |
| PLAGL2 | EAF2 |
| PLAT | PLAT |
| PLAUR | RRAS |
| PLEC | EGFR |
| PLEC | LAMB3 |
| PLEC | OSMR |
| PLEC | S100A16 |
| PLEK2 | DDR1 |
| PLEK2 | TNFRSF21 |
| PLEKHA6 | ELF3 |
| PLEKHA7 | RASSF7 |
| PLEKHA8 | PLEKHA8 |
| PLEKHB1 | PLEKHB1 |
| PLEKHG6 | MAL2 |
| PLEKHJ1 | RNF126 |
| PLEKHO1 | SYT11 |
| PLK2 | CTNNA1 |
| PLK2 | IL6ST |
| PLOD3 | CALU |
| PLXDC2 | PLXDC2 |
| PLXNA1 | DIRC2 |
| PMCH | TROAP |
| PMEPA1 | KRT80 |
| PMM1 | CYB5R3 |
| PMPCA | MRPS2 |
| PMPCA | URM1 |
| PNKP | PNKP |
| PNKP | STRN4 |
| PNN | HNRNPC |
| PNO1 | ACP1 |
| PNO1 | SSB |
| PNPLA2 | PNPLA2 |
| PNPLA6 | PGL5 |
| POC5 | TAF9 |
| POGK | ANGEL2 |
| POGK | CNBP |
| POGK | USP21 |
| POGZ | ARNT |
| POGZ | PYGO2 |
| POGZ | ZNF678 |
| POLD1 | LIG1 |
| POLD1 | ZNF611 |
| POLDIP3 | GTPBP1 |
| POLDIP3 | L3MBTL2 |
| POLE2 | L2HGDH |
| POLE2 | TOP2A |
| POLG2 | BPTF |
| POLG2 | C2orf44 |
| POLG2 | COIL |
| POLG2 | DCAF7 |
| POLG2 | PTCD3 |
| POLG2 | RPL23 |
| POLI | TXNL1 |
| POLK | PJA2 |
| POLR1D | POLR1D |
| POLR2A | WRAP53 |
| POLR2C | COX4NB |
| POLR2E | CDC34 |
| POLR2E | RNF126 |
| POLR2E | SLC39A3 |
| POLR2F | L3MBTL2 |
| POLR2G | C11orf48 |
| POLR2J4 | KRIT1 |
| POLR2K | ARPC5 |
| POLR2K | HRSP12 |
| POLR2K | NDUFB5 |
| POLR2K | PRKDC |
| POLR2K | UQCRB |
| POLR3D | TRIM35 |
| POLR3F | ATRN |
| POLR3F | SEC23B |
| POLR3K | ZNF174 |
| POMGNT1 | POMGNT1 |
| PON2 | PTPN12 |
| POP7 | NDUFB2 |
| POP7 | POP7 |
| POR | FASTK |
| POU2F1 | USP21 |
| POU2F1 | ZNF678 |
| POU5F2 | POU6F2 |
| PPAN | GTPBP3 |
| PPAPDC1B | PPAPDC1B |
| PPAPDC2 | AK3 |
| PPCS | TNNI3K |
| PPFIBP1 | PHLDA1 |
| PPIA | PPIA |
| PPIB | TMED3 |
| PPIC | SNX24 |
| PPIF | PPIF |
| PPIH | FAF1 |
| PPIH | PPIH |
| PPIL2 | PI4KA |
| PPIP5K1 | PPIP5K1 |
| PPIP5K2 | SNX2 |
| PPM1A | KLHL28 |
| PPM1D | BPTF |
| PPM1D | PPM1D |
| PPP1CC | CCDC59 |
| PPP1CC | NFYB |
| PPP1R15B | C1orf55 |
| PPP1R2 | MRPL3 |
| PPP1R2 | RNF13 |
| PPP1R2 | SENP2 |
| PPP1R3A | GRM8 |
| PPP1R8 | DNAJC8 |
| PPP1R8 | HNRNPR |
| PPP1R8 | NASP |
| PPP2CA | CANX |
| PPP2CA | CSNK1G3 |
| PPP2CA | GIN1 |
| PPP2R2A | CNOT7 |
| PPP2R2A | ELP3 |
| PPP2R3A | AMOTL2 |
| PPP2R3A | FEZ2 |
| PPP2R3A | OSMR |
| PPP2R5C | ATXN3 |
| PPP2R5C | PAPOLA |
| PPP2R5D | TUBB |
| PPP5C | LIG1 |
| PPP5C | PRMT1 |
| PPP5C | SAE1 |
| PPP6C | GAPVD1 |
| PPP6C | POLE3 |
| PPPDE2 | PPPDE2 |
| PPWD1 | CHD1 |
| PPWD1 | CWC27 |
| PPWD1 | NDUFS4 |
| PPWD1 | RIOK2 |
| PPWD1 | TAF9 |
| PRDM10 | BUD13 |
| PRDM10 | NFRKB |
| PRDM2 | ARID1A |
| PRDX2 | PDE4C |
| PRDX3 | ERLIN1 |
| PRDX3 | NSMCE4A |
| PRDX3 | PPA1 |
| PRDX3 | XPNPEP1 |
| PRDX5 | PRDX5 |
| PRELID1 | UTP15 |
| PRIM1 | DDX11 |
| PRKAA1 | ITCH |
| PRKAA1 | NMD3 |
| PRKAA1 | PRKAA1 |
| PRKAA1 | UGP2 |
| PRKAB2 | PRKAB2 |
| PRKAR2B | PRKAR2B |
| PRKD1 | CFL2 |
| PRKD2 | STRN4 |
| PRKDC | PARP1 |
| PRNP | ARPC1A |
| PRNP | ATP6V1C1 |
| PRNP | DLG1 |
| PRNP | IGFBP6 |
| PROCR | ASPH |
| PRPF18 | ARPC5 |
| PRPF18 | MLLT10 |
| PRPF18 | POLR2K |
| PRPF19 | C11orf48 |
| PRPF3 | SCNM1 |
| PRPF31 | FIZ1 |
| PRPF31 | MRPS12 |
| PRPF31 | NDUFA3 |
| PRPF31 | NUP62 |
| PRPF31 | POLD1 |
| PRPF31 | TRIM28 |
| PRPF31 | ZNF576 |
| PRPF38A | PPIH |
| PRPF39 | C14orf166 |
| PRPF39 | METTL3 |
| PRPF4 | IKBKAP |
| PRPF4 | PMPCA |
| PRPF8 | GSG2 |
| PRR11 | MAP3K3 |
| PRR14 | NFATC2IP |
| PRR14 | PRR14 |
| PRR14 | USP7 |
| PRR14 | ZNF668 |
| PRR15 | CLDN4 |
| PRR15 | KLF5 |
| PRR3 | MDC1 |
| PRR3 | PARP1 |
| PRR3 | RIOK1 |
| PRRG2 | EPS8L1 |
| PRSS3 | PRSS3 |
| PRSS8 | ELF3 |
| PRSS8 | LAD1 |
| PRSS8 | SLPI |
| PRTFDC1 | PRTFDC1 |
| PRUNE | ARNT |
| PSMA1 | CAPRIN1 |
| PSMA2 | CHCHD2 |
| PSMA2 | H2AFV |
| PSMA2 | MRPL32 |
| PSMA3 | EIF5 |
| PSMA3 | VTI1B |
| PSMB3 | AATF |
| PSMB3 | NME1 |
| PSMC5 | NME1 |
| PSMD10 | NXT2 |
| PSMD12 | CCT5 |
| PSMD12 | KLHL12 |
| PSMD12 | PSMD11 |
| PSMD12 | SLC35B1 |
| PSMD12 | SRP9 |
| PSMD13 | PSMA1 |
| PSMD6 | ATG3 |
| PSMD6 | PDHB |
| PSME3 | DNAJC7 |
| PSMF1 | RBCK1 |
| PSMG1 | RRP1B |
| PSRC1 | COCA8 |
| PTBP1 | RNF126 |
| PTBP2 | LRRC40 |
| PTBP2 | MTF2 |
| PTBP2 | PTBP2 |
| PTCD2 | PTCD2 |
| PTCD2 | TAF9 |
| PTCH1 | PTCH1 |
| PTGES | CLIC3 |
| PTGFR | PTGFR |
| PTGIS | PTGIS |
| PTGR2 | PTGR2 |
| PTK2 | PSMD12 |
| PTK6 | ATP2C2 |
| PTK6 | ESRP2 |
| PTK6 | KLF5 |
| PTK6 | KRT8 |
| PTN | PTN |
| PTOV1 | FKBP8 |
| PTOV1 | PNKP |
| PTPLAD1 | RCN2 |
| PTPN2 | PTPN2 |
| PTPN21 | CFL2 |
| PTPRF | CLDN1 |
| PTPRF | EGFR |
| PTPRK | DDR1 |
| PTTG1 | HMMR |
| PUM1 | KDM1A |
| PUM1 | SFPQ |
| PUM2 | INO80D |
| PVRL4 | EVPL |
| PVRL4 | GRHL2 |
| PVRL4 | LAD1 |
| PWP2 | C21orf59 |
| PYGO2 | ITGB1 |
| QRICH1 | PDE12 |
| R3HCC1 | CNOT7 |
| RAB11A | RAB11A |
| RAB11FIP1 | MAL2 |
| RAB11FIP5 | NCEH1 |
| RAB14 | GAPVD1 |
| RAB1A | ATG3 |
| RAB1A | PIGF |
| RAB1A | PNO1 |
| RAB1A | PRKAA1 |
| RAB1A | RNF13 |
| RAB1A | UBXN4 |
| RAB20 | CLDN4 |
| RAB20 | RAB20 |
| RAB22A | ARPC1A |
| RAB22A | C20orf24 |
| RAB23 | SEC23A |
| RAB23 | VAMP7 |
| RAB25 | LAD1 |
| RAB25 | SDR16C5 |
| RAB2A | ASPH |
| RAB34 | PTRF |
| RAB34 | RAB34 |
| RAB38 | CTSC |
| RAB3A | RAB3A |
| RAB3B | RAB3B |
| RAD1 | RAD1 |
| RAD17 | GDE1 |
| RAD17 | TAF9 |
| RAD18 | DYNC1LI1 |
| RAD21 | CCNE2 |
| RAD21 | DSCC1 |
| RAD23A | FARSA |
| RAD23B | GTF3C4 |
| RAD23B | NCBP1 |
| RAD23B | SPTLC1 |
| RAD50 | RAD50 |
| RAD51AP1 | CDK2 |
| RAD51AP1 | POLQ |
| RAD51AP1 | TMPO |
| RAD51C | CCT2 |
| RAD51C | NME1 |
| RAE1 | PDRG1 |
| RAF1 | WDR48 |
| RAI14 | FSTL1 |
| RAI14 | LEPREL1 |
| RAI14 | MET |
| RAI14 | OSMR |
| RAI14 | TIMP2 |
| RALY | C20orf4 |
| RALY | PCIF1 |
| RANBP1 | SNRPD3 |
| RANBP2 | SSB |
| RANBP3 | ADAT3 |
| RANBP3 | FARSA |
| RANBP3 | GTPBP3 |
| RANBP3 | MBD3 |
| RANBP3 | MLLT1 |
| RANBP3 | PIN1 |
| RANBP3 | POLRMT |
| RANBP3 | RAVER1 |
| RANBP3 | WDR18 |
| RANBP6 | PSIP1 |
| RAP1GDS1 | RAP1GD51 |
| RARS | SNX2 |
| RARS | TAF9 |
| RASA1 | GIN1 |
| RASAL2 | OSMR |
| RASSF5 | RASSF5 |
| RB1CC1 | PRKDC |
| RB1CC1 | TCEB1 |
| RBAK | RBAK |
| RBBP4 | ITGB3BP |
| RBL1 | E2F1 |
| RBL1 | MCM7 |
| RBM10 | PHF8 |
| RBM12 | SSB |
| RBM12 | ZBTB39 |
| RBM12B | LUC7L3 |
| RBM12B | WDR67 |
| RBM14 | SF1 |
| RBM15 | FUBP1 |
| RBM15 | RAD54L |
| RBM17 | ANKRD16 |
| RBM17 | SUV39H2 |
| RBM18 | NDUFA8 |
| RBM26 | EXOSC8 |
| RBM26 | USPL1 |
| RBM33 | EZH2 |
| RBM33 | ZNF212 |
| RBM34 | C1orf55 |
| RBM39 | ADNP |
| RBM39 | CCNL1 |
| RBM4 | MEN1 |
| RBM7 | FDX1 |
| RBPMS | ASPH |
| RC3H1 | MDM4 |
| RCE1 | RCE1 |
| RCHY1 | RCHY1 |
| RCOR3 | ARID4B |
| RCOR3 | SRP9 |
| RECQL4 | PYCRL |
| REM2 | REM2 |
| REPS1 | REPS1 |
| RER1 | AURKAIP1 |
| RERE | UBE4B |
| RFC1 | NUP54 |
| RFC4 | MCM8 |
| RFC4 | MRPL47 |
| RFC4 | NDC80 |
| RFK | RFK |
| RFX5 | TARS2 |
| RGMA | RGMA |
| RGS6 | RGS6 |
| RHBDF1 | METRN |
| RHBDF1 | TNFRSF12A |
| RHBDL2 | EGFR |
| RHBDL2 | S100A16 |
| RHOC | NOTCH2 |
| RHOC | POMGNT1 |
| RHOD | RHOD |
| RHOD | TSKU |
| RHOG | TAF10 |
| RILPL1 | CKAP4 |
| RIMS3 | KHDRBS1 |
| RIN2 | ASPH |
| RIN2 | KRT7 |
| RIN2 | SRGAP1 |
| RINT1 | EIF4H |
| RINT1 | POT1 |
| RIOK1 | PAK1IP1 |
| RIOK1 | PRR3 |
| RIOK2 | GIN1 |
| RIOK2 | TAF9 |
| RIOK3 | MYL12A |
| RIOK3 | UGP2 |
| RNF11 | POMGNT1 |
| RNF11 | RNF11 |
| RNF121 | IL18BP |
| RNF126 | NCLN |
| RNF126 | RNF126 |
| RNF138 | HDHD2 |
| RNF138 | TXNL1 |
| RNF139 | RNF139 |
| RNF14 | AGGF1 |
| RNF14 | AP3B1 |
| RNF14 | GNS |
| RNF20 | RNF20 |
| RNF219 | BRCA2 |
| RNF219 | CUL4A |
| RNF219 | EXOSC8 |
| RNF219 | IPO5 |
| RNF220 | GPBP1L1 |
| RNF25 | RNF25 |
| RNF26 | DPAGT1 |
| RNF40 | MAPKS8IP3 |
| RNF44 | ZBTB39 |
| RNF6 | CDK8 |
| RNF6 | MTIF3 |
| RNGTT | HDAC2 |
| RNH1 | TAF10 |
| RNPS1 | E4F1 |
| RPA3 | CHCHD2 |
| RPA3 | UBE2C |
| RPAP1 | RPAP1 |
| RPAP3 | PPP1CC |
| RPF1 | BCAS2 |
| RPF1 | CDC7 |
| RPF1 | GLMN |
| RPF1 | LRRC40 |
| RPF1 | MTF2 |
| RPF1 | RWDD3 |
| RPF1 | TAF12 |
| RPH3A | RPH3A |
| RPL13A | C19orf48 |
| RPL14 | CNOT10 |
| RPL14 | IMPDH2 |
| RPL30 | UBR5 |
| RPL35A | NACA |
| RPL35A | RPL24 |
| RPL35A | RPS27A |
| RPL36 | RPS15 |
| RPL38 | BPTF |
| RPL4 | CLPX |
| RPL4 | CSK |
| RPL4 | DENND4A |
| RPL8 | EIF2C2 |
| RPP38 | ANKRD16 |
| RPRD1A | HDHD2 |
| RPRD1B | YTHDF1 |
| RPRD2 | ARNT |
| RPS15 | EIF3E |
| RPS23 | TAF9 |
| RPS6KA4 | CAPN1 |
| RPS6KB1 | ZBTB11 |
| RPS6KB1 | ZNF207 |
| RPS6KB2 | PTPRCAP |
| RPUSD2 | AQR |
| RPUSD2 | IMP3 |
| RPUSD3 | THUMPD3 |
| RRAGA | KLHL9 |
| RRAS | MBOAT7 |
| RRAS | RRAS |
| RRBP1 | CALU |
| RRM2B | MDM2 |
| RRP1B | SLC19A1 |
| RRP1B | UBE2G2 |
| RSBN1L | EZH2 |
| RSL1D1 | USP7 |
| RSL24D1 | IREB2 |
| RSU1 | VIM |
| RTCD1 | RTCD1 |
| RTEL1 | TNFRSF6B |
| RTN4 | FEZ2 |
| RTP1 | RTP1 |
| RYK | TBL1XR1 |
| S100A1 | S100A1 |
| S100A10 | ABCC3 |
| S100A10 | LMNA |
| S100A10 | OSMR |
| S100A11 | ELF3 |
| S100A11 | OSMR |
| S100A13 | S100A6 |
| S100A14 | C1orf106 |
| S100A14 | S100A16 |
| S100A14 | SDR16C5 |
| S100A6 | ABCC3 |
| S100A6 | ITGA3 |
| S100A6 | QSOX1 |
| S100A6 | SERPINB6 |
| SAC3D1 | NAA40 |
| SAE1 | BCL2L12 |
| SAE1 | LIG1 |
| SAE1 | PRMT1 |
| SAFB2 | MAST3 |
| SALL1 | SALL1 |
| SAMD1 | RAVER1 |
| SAMD4A | MICA |
| SAMD4A | PTPN21 |
| SAP30BP | NUP85 |
| SART3 | RNF34 |
| SART3 | SENP1 |
| SASS6 | PTBP2 |
| SASS6 | TMEM48 |
| SBF1 | ZC3H7B |
| SCAMP4 | FASTK |
| SCAMP4 | MBD3 |
| SCAMP4 | PIP5K1C |
| SCFD1 | TMED10 |
| SCO2 | TYMP |
| SCP2 | RNF11 |
| SCRIB | PYCRL |
| SCRIB | ZFP41 |
| SCYL2 | PTGES3 |
| SCYL2 | SCYL2 |
| SCYL2 | STRAP |
| SDC4 | ASPH |
| SDC4 | CD9 |
| SDC4 | EGFR |
| SDC4 | EPB41L1 |
| SDC4 | GPR39 |
| SDC4 | KRT8 |
| SDC4 | OSMR |
| SDCCAG3 | GTF3C4 |
| SDHAF1 | U2AF1L4 |
| SDHC | ADIPOR1 |
| SDHC | HRSP12 |
| SDHC | PSMD12 |
| SEC11A | SEC11A |
| SEC11C | TXNL1 |
| SEC23A | RAB23 |
| SEC23IP | NRBF2 |
| SEC24A | SAR1B |
| SEC24C | BMS1P5 |
| SEC61A1 | SEC61A1 |
| SEH1L | RNMT |
| SEL1L | SGPP1 |
| SELT | ACP1 |
| SELT | B3GNT2 |
| SELT | MED21 |
| SELT | RAB21 |
| SELT | SLC33A1 |
| SELT | TOMM22 |
| SELT | TPRKB |
| SEMA3C | IGFBP3 |
| SENP1 | NFYB |
| SENP1 | YEATS4 |
| SENP2 | ACP1 |
| SENP2 | BAG2 |
| SENP2 | DNM1L |
| SENP2 | GPR89B |
| SENP2 | GTF3C3 |
| SENP2 | MRPL3 |
| SENP2 | RAB23 |
| SENP2 | RPS6KB1 |
| SENP2 | STRAP |
| SENP2 | TFG |
| SENP2 | TOMM22 |
| SENP2 | UBXN4 |
| SENP2 | UGP2 |
| SENP5 | SENP5 |
| SENP6 | SENP6 |
| SENP7 | TBL1XR1 |
| SEPT6 | SEPT6 |
| SERBP1 | RBBP4 |
| SERBP1 | RBM8A |
| SERBP1 | SF3A3 |
| SERBP1 | TRIM33 |
| SERINC1 | ECHDC1 |
| SERINC2 | INADL |
| SERPIND1 | SERPIND1 |
| SERPINE1 | DFNA5 |
| SERPINE1 | INHBA |
| SET | STRBP |
| SETBP1 | SETBP1 |
| SETD5 | WDR48 |
| SETDB1 | ARNT |
| SETDB1 | MBTD1 |
| SETDB1 | ZNF33A |
| SF1 | MEN1 |
| SF1 | PRPF19 |
| SF3A1 | DRG1 |
| SF3A3 | RBBP4 |
| SF3B1 | TIA1 |
| SF3B3 | DHX38 |
| SF3B3 | KARS |
| SF3B3 | PRMT7 |
| SFI1 | ZC3H7B |
| SFN | AGRN |
| SFN | EGFR |
| SFN | PTPRF |
| SFXN4 | ATE1 |
| SFXN4 | NSMCE4A |
| SGMS1 | ADK |
| SGPL1 | DLG5 |
| SGPP1 | EXOC5 |
| SGSH | SPATA20 |
| SGSM2 | SHPK |
| SGSM3 | GGA1 |
| SGTA | RNF125 |
| SH2B1 | PRR14 |
| SH2B1 | UBN1 |
| SH3D19 | FAT1 |
| SHCBP1 | PLK1 |
| SHMT1 | SHMT1 |
| SHPRH | BCLAF1 |
| SIAH2 | PIK3CA |
| SIKE1 | HIPK1 |
| SIL1 | SQSTM1 |
| SIN3B | CARM1 |
| SIN3B | SUPT5H |
| SIRPB2 | SIRPB2 |
| SKIV2L2 | CHD1 |
| SKIV2L2 | CWC27 |
| SKIV2L2 | RIOK2 |
| SKIV2L2 | TAF9 |
| SKP2 | KIF14 |
| SKP2 | RAD1 |
| SLA | CD1C |
| SLAMF1 | RGS1 |
| SLAMF6 | FMO2 |
| SLC10A3 | IKBKG |
| SLC19A2 | SLC19A2 |
| SLC20A2 | SLC20A2 |
| SLC22A12 | SLC22A12 |
| SLC22A4 | OSMR |
| SLC25A11 | RNF167 |
| SLC25A19 | GGA3 |
| SLC25A19 | TAF4B |
| SLC25A25 | SLC25A25 |
| SLC25A32 | ENY2 |
| SLC25A32 | HRSP12 |
| SLC25A32 | IMPA1 |
| SLC25A36 | ZNF148 |
| SLC25A38 | PDE12 |
| SLC25A38 | RBM6 |
| SLC25A40 | CASP2 |
| SLC2SA40 | PAXIP1 |
| SLC2SA40 | SLC25A40 |
| SLC25A44 | PI4KB |
| SLC25A5 | SLC25A5 |
| SLC29A3 | SLC29A3 |
| SLC2A1 | BCAR3 |
| SLC2A1 | S100A2 |
| SLC2A10 | SLC2A10 |
| SLC30A5 | GIN1 |
| SLC30A5 | RARS |
| SLC30A5 | SNX2 |
| SLC30A5 | TAF9 |
| SLC35B3 | SLC35B3 |
| SLC37A2 | SLC37A2 |
| SLC37A4 | SLC37A4 |
| SLC39A13 | CD151 |
| SLC39A13 | DKK3 |
| SLC39A3 | RNF126 |
| SLC43A3 | SLC43A3 |
| SLC44A3 | PTPRF |
| SLC5A12 | SLC5A12 |
| SLC6A11 | SLC6A11 |
| SLC7A13 | SLC7A13 |
| SLC7A14 | SLC7A14 |
| SLC8A2 | SLC8A2 |
| SLCO1C1 | CLEC1A |
| SLK | VPS26A |
| SLTM | IREB2 |
| SMAD2 | TXNL1 |
| SMAD3 | EGFR |
| SMAD4 | LMAN1 |
| SMARCA2 | JAK2 |
| SMARCA4 | AKAP8L |
| SMARCA4 | HNRNPM |
| SMARCB1 | GTSE1 |
| SMARCD2 | DCAF7 |
| SMC4 | BUB1 |
| SMC4 | RACGAP1 |
| SMC6 | CNBP |
| SMCHD1 | VAPA |
| SMCHD1 | ZNF519 |
| SMCR7L | ACO2 |
| SMCR7L | L3MBTL2 |
| SMCR7L | TNRC6B |
| SMEK1 | UBR7 |
| SMEK2 | B3GNT2 |
| SMEK2 | C2orf29 |
| SMURF2 | OSMR |
| SNAP29 | MAPK1 |
| SNAP29 | PI4KA |
| SNAPC1 | CFL2 |
| SNAPC4 | USP20 |
| SNCG | SNCG |
| SNHG1 | RPS3 |
| SNHG4 | SNX2 |
| SNHG4 | TAF9 |
| SNHG7 | DDX31 |
| SNORA25 | CUL5 |
| SNORA25 | RPS3 |
| SNORA72 | UBR5 |
| SNRNP25 | NDUFB10 |
| SNRNP40 | KDM1A |
| SNRNP70 | BCL2L12 |
| SNRNP70 | IRF2BP1 |
| SNRPA | XRCC1 |
| SNRPD1 | ATP5A1 |
| SNRPD2 | LIG1 |
| SNW1 | ERH |
| SNW1 | PAPOLA |
| SNX1 | ARIH1 |
| SNX1 | PIGB |
| SNX11 | SNX11 |
| SNX2 | AP3B1 |
| SNX2 | CSNK1G3 |
| SNX2 | GIN1 |
| SNX2 | TRIM23 |
| SNX2 | UBC |
| SNX24 | SNX24 |
| SNX33 | ANXA2 |
| SMX4 | SNX4 |
| SNX6 | SNX6 |
| SNX7 | ARHGAP29 |
| SNX7 | JUN |
| SOCS2 | SOCS2 |
| SOCS4 | EXOC5 |
| SOS1 | SOS1 |
| SOX10 | SOX10 |
| SOX9 | ABCC3 |
| SOX9 | SOX9 |
| SPARC | GPX8 |
| SPARC | PCDHGC5 |
| SPAST | KIDINS220 |
| SPATA5 | MAD2L1 |
| SPATA7 | SPATA7 |
| SPEN | ARID1A |
| SPEN | HNRNPR |
| SPINK6 | SPINK6 |
| SPINT2 | EP58L1 |
| SPINT2 | SLPI |
| SPINT2 | SPINT2 |
| SPRR4 | AIF1 |
| SPSB3 | E4F1 |
| SPTA1 | SPTA1 |
| SPTLC1 | DNAJC25-GNG10 |
| SQSTM1 | GNS |
| SQSTM1 | LHFPL2 |
| SQSTM1 | MET |
| SQSTM1 | TGFBI |
| SRCAP | SETD1A |
| SREBF2 | GTPBP1 |
| SREBF2 | SREBF2 |
| SRPX2 | EGFR |
| SRRT | EZH2 |
| SS18L2 | CCDC12 |
| SS18L2 | CNOT10 |
| SS18L2 | KLHL18 |
| SS18L2 | MLH1 |
| SSBP1 | POP7 |
| SSH3 | RHOD |
| SSH3 | TSKU |
| SSNA1 | GTF3C5 |
| ST14 | MPZL2 |
| ST14 | RHOD |
| ST14 | ST14 |
| STAC3 | STAC3 |
| STAG2 | ZNF280C |
| STAMBP | GTF3C3 |
| STARD10 | FOXA1 |
| STAT3 | STAT3 |
| STEAP4 | EPHA1 |
| STIL | STIL |
| STIP1 | TMEM126B |
| STOML2 | SIGMAR1 |
| STRN3 | HECTD1 |
| STRN3 | MBIP |
| STRN4 | GPATCH1 |
| STRN4 | PNKP |
| STRN4 | XRCC1 |
| STUB1 | AMDHD2 |
| STUB1 | STUB1 |
| STX10 | FARSA |
| STX11 | STX11 |
| STX12 | STX12 |
| STX3 | STX3 |
| STX8 | STX8 |
| STX8 | TRAPPC1 |
| STXBP3 | HBXIP |
| STXBP3 | RWDD3 |
| STYK1 | GPRC5A |
| SUB1 | RAD1 |
| SUCLG2 | SUCLG2 |
| SUDS3 | MLL2 |
| SUDS3 | SBNO1 |
| SUN1 | RAC1 |
| SUPT5H | GPATCH1 |
| SUPT5H | IRF2BP1 |
| SUPT6H | GGA3 |
| SURF1 | SNAPC4 |
| SURF2 | GTF3C4 |
| SURF6 | GTF3C4 |
| SUV39H2 | KIF11 |
| SV2A | SYT11 |
| SVOPL | SVOPL |
| SYDE1 | CALU |
| SYDE1 | FSTL3 |
| SYMPK | IRF2BP1 |
| SYNCRIP | HSF2 |
| SYNCRIP | SENP6 |
| SYNJ2BP | BCL2L2 |
| SYNM | SYNM |
| SYT11 | ATP8B2 |
| SYT11 | SYT11 |
| SYT2 | SYT2 |
| TAB2 | RNF146 |
| TACC2 | KIAA1598 |
| TACC2 | PLEKHA1 |
| TACO1 | MRPL27 |
| TACSTD2 | LIPH |
| TADA1 | GPLD1 |
| TADA1 | MBTD1 |
| TADA1 | ZNF672 |
| TADA2A | AATF |
| TAF1 | RLIM |
| TAF1D | RPS25 |
| TAF2 | DSCC1 |
| TAF2 | UBR5 |
| TAF4B | LMAN1 |
| TAF7 | TAF7 |
| TAF9 | GIN1 |
| TAF9 | PTCD2 |
| TAF9 | TAF9 |
| TAOK1 | USP36 |
| TAOK2 | AMDHD2 |
| TAOK2 | PRR14 |
| TAOK2 | RABEP2 |
| TARBP2 | CDK4 |
| TAS2R7 | TAS2R7 |
| TAS2R9 | MC3R |
| TAX1BP1 | YKT6 |
| TAZ | IDH3G |
| TAZ | IKBKG |
| TBC1D10B | MAZ |
| TBC1D10B | ZNF335 |
| TBC1D10B | ZNF771 |
| TBC1D13 | FPGS |
| TBC1D2 | ANXA1 |
| TBC1D5 | C3orf19 |
| TBC1D9B | MGAT4B |
| TBCE | FH |
| TBL1XR1 | CMAS |
| TBL1XR1 | DNM1L |
| TBL1XR1 | MAPK1 |
| TBL1XR1 | MRPL3 |
| TBL1XR1 | TBL1XR1 |
| TBL1XR1 | TOMM22 |
| TBL1XR1 | UBA5 |
| TBL3 | PMM2 |
| TBL3 | TAOK2 |
| TBL3 | TSC2 |
| TBP | ADAT2 |
| TBP | ARID1B |
| TBRG4 | AVL9 |
| TBX3 | TBX3 |
| TC2N | FOXA1 |
| TCEA2 | TCEA2 |
| TCEAL1 | PSMD10 |
| TCEAL1 | TCEAL4 |
| TCEAL4 | TCEAL4 |
| TCEAL8 | WBP5 |
| TCEB1 | ZFAND1 |
| TCERG1 | PPWD1 |
| TCERG1 | RAPGEF6 |
| TCF20 | TCF20 |
| TCF21 | TCF21 |
| TCFL5 | TCFL5 |
| TCL1A | TCL1A |
| TCL6 | TCL1A |
| TCOF1 | LARP1 |
| TCP1 | BCLAF1 |
| TCP1 | FAM54A |
| TCP1 | FBXO5 |
| TDP1 | DLGAP5 |
| TDP1 | PAPOLA |
| TELO2 | E4F1 |
| TELO2 | MAZ |
| TELO2 | PDPK1 |
| TELO2 | ZNF500 |
| TELO2 | ZNF771 |
| TERF2 | CBFB |
| TERF2 | CTCF |
| TERF2IP | TERF2IP |
| TEX10 | POLE3 |
| TFAP2C | TFAP2C |
| TFDP1 | CCNE2 |
| TFDP1 | RFC3 |
| TFDP1 | TFDP1 |
| TFF1 | TFF1 |
| TFG | IL20RB |
| TFG | SEPT10 |
| TFIP11 | TRMT2A |
| TGFBI | DAB2 |
| TGFBI | PLK2 |
| TGM6 | TGM6 |
| TH | TH |
| THAP11 | KARS |
| THOC1 | THOC1 |
| THOC2 | PHF6 |
| THOC6 | THOC6 |
| THOC7 | RPL14 |
| THOP1 | RNF126 |
| THYN1 | ACAD8 |
| TIFA | C4orf21 |
| TIMELESS | DDX11 |
| TIMELESS | TMPO |
| TIMELESS | ZBTB39 |
| TIMM17A | FH |
| TIMM17A | HRSP12 |
| TIMM17B | GPKOW |
| TIMM44 | RNF126 |
| TIMM88 | ATP5L |
| TIPRL | TIPRL |
| TK2 | CES2 |
| TLCD1 | TRAF4 |
| TLK1 | B3GNT2 |
| TLK1 | CREB1 |
| TLK2 | COIL |
| TLN1 | TLN1 |
| TLX3 | TLX3 |
| TM4SF1 | ANXA4 |
| TM4SF1 | EGFR |
| TM4SF1 | GPRC5A |
| TM4SF1 | KDELR3 |
| TM4SF1 | LPP |
| TM4SF1 | OSMR |
| TM9SF1 | BCL2L2 |
| TMCC2 | TMCC2 |
| TMCO1 | GDE1 |
| TMCO1 | GPR89B |
| TMED10 | TM9SF1 |
| TMED2 | CMAS |
| TMED2 | KIAA1033 |
| TMED5 | SCP2 |
| TMEM106B | RAC1 |
| TMEM111 | ATG7 |
| TMEM115 | GLT8D1 |
| TMEM115 | SEC13 |
| TMEM116 | TMEM116 |
| TMEM120A | BRI3 |
| TMEM125 | TACSTD2 |
| TMEM134 | RAB1B |
| TMEM135 | DLAT |
| TMEM135 | MED17 |
| TMEM14B | TMEM14B |
| TMEM161A | AKAP8 |
| TMEM161A | FARSA |
| TMEM161A | GTPBP3 |
| TMEM17 | EHBP1 |
| TMEM18 | TMEM18 |
| TMEM184B | KDELR3 |
| TMEM184B | MICALL1 |
| TMEM184B | PLXNB2 |
| TMEM186 | USP7 |
| TMEM194A | CAND1 |
| TMEM194A | TMPO |
| TMEM199 | SPAG5 |
| TMEM203 | GTF3C5 |
| TMEM212 | TACR1 |
| TMEM217 | TMEM217 |
| TMEM222 | DNAJC8 |
| TMEM223 | MRPL49 |
| TMEM33 | NFXL1 |
| TMEM39B | DNAJC8 |
| TMEM45B | ST14 |
| TMEM59 | RNF11 |
| TMEM70 | ZFAND1 |
| TMEM93 | RNF167 |
| TMEM97 | E2F1 |
| TMPO | CDCA3 |
| TMPO | MPHOSPH9 |
| TMPO | RFC5 |
| TMPO | SENP1 |
| TMX1 | MED6 |
| TNFRSF12A | TGFB1I1 |
| TNFRSF1A | LPP |
| TNFRSF6B | TNFRSF6B |
| TNKS | HMBOX1 |
| TNPO2 | CARM1 |
| TNPO2 | FARSA |
| TNPO2 | SMARCA4 |
| TNR | CA1 |
| TN53 | LGALS3 |
| TN54 | JUP |
| TOM1L1 | TOM1L1 |
| TOPBP1 | MSH2 |
| TOPBP1 | RANBP1 |
| TOR1AIP1 | ADSS |
| TOR1AIP1 | ARPC5 |
| TP53INP1 | BTG2 |
| TPBG | PTPRK |
| TPD52L1 | DDR1 |
| TPP2 | EXOSC8 |
| TPP2 | UPF3A |
| TPRKB | ACP1 |
| TP5T1 | DFNA5 |
| TPX2 | ECT2 |
| TPX2 | SKP2 |
| TPX2 | XPO1 |
| TRA2B | RANBP1 |
| TRA2B | TSN |
| TRABD | TRMT2A |
| TRAF2 | GTF3C5 |
| TRAM1 | HRSP12 |
| TRAPPC6B | SOS2 |
| TRAT1 | TRAT1 |
| TRERF1 | TRERF1 |
| TRIB3 | RBCK1 |
| TRIM23 | GDE1 |
| TRIM23 | TAF9 |
| TRIM24 | LUC7L2 |
| TRIM28 | GPATCH1 |
| TRIM28 | LIG1 |
| TRIM28 | PNKP |
| TRIM29 | ST14 |
| TRIM35 | BIN3 |
| TRIM35 | PPP3CC |
| TRIM41 | ZFP62 |
| TRIM52 | ZFP62 |
| TRIOBP | PLXNB2 |
| TRIP12 | GIGYF2 |
| TRIP13 | SPAG5 |
| TRIP6 | PLOD3 |
| TRMT1 | ATP13A1 |
| TRMT1 | TNPO2 |
| TRMT11 | ADAT2 |
| TRMT11 | HDAC2 |
| TRMT12 | RNF139 |
| TRMT2A | GTPBP1 |
| TRMT5 | MNAT1 |
| TRNAU1AP | DNAJC8 |
| TRNT1 | TSEN2 |
| TROVE2 | ARID4B |
| TRRAP | LUC7L2 |
| TRUB2 | MRRF |
| TSC2 | E4F1 |
| TSC2 | STUB1 |
| TSC2 | ZNF500 |
| TSC22D3 | TSC22D3 |
| TSEN54 | MRPL12 |
| TSN | SENP2 |
| TSN | XRCC5 |
| TSNAX | LIN9 |
| TSPAN13 | CLDN4 |
| TSPAN13 | FOXA1 |
| TSTA3 | PVCRL |
| TSTA3 | SLC39A4 |
| TSTD1 | ELF3 |
| TSTD2 | PHF2 |
| TTC3 | TTC3 |
| TTC35 | DERL1 |
| TTC37 | TAF9 |
| TTC78 | TTC78 |
| TTF1 | EHMT1 |
| TTLL5 | C14orf1 |
| TUBA1A | CBX5 |
| TUBB | TUBB |
| TUBB6 | CRIM1 |
| TUBD1 | COIL |
| TUBGCP3 | BRCA2 |
| TUBGCP5 | RTF1 |
| TUFT1 | EDN1 |
| TUFT1 | ELF3 |
| TUFT1 | MAL2 |
| TUFT1 | TUFT1 |
| TUT1 | SF1 |
| TXLNA | DNAJC8 |
| TXNDC16 | UBR7 |
| TYK2 | ATP13A1 |
| TYK2 | RANBP3 |
| TYK2 | RAVER1 |
| TYMS | THOC1 |
| UBA3 | ATXN7 |
| UBA5 | TSL1XR1 |
| UBA52 | ZNF101 |
| UBA6 | LARP7 |
| UBASH3B | UBASH3B |
| UBE2C | DNTTIP1 |
| UBE2C | ECT2 |
| UBE2C | NCAPD2 |
| UBE2H | ARPC1A |
| UBE2H | IFRD1 |
| UBE2M | TRIM28 |
| UBE2N | SCYL2 |
| UBE2N | ZDHHC17 |
| UBE2O | RECQL5 |
| UBE2O | UBTF |
| UBE2O | USP36 |
| UBE2Q1 | PYGO2 |
| UBE2T | CCNE2 |
| UBE2V2 | MTFR1 |
| UBL4A | IKBKG |
| UBN1 | DNASE1L2 |
| UBN1 | E4F1 |
| UBNI | USP7 |
| UBN1 | ZNF500 |
| UBN2 | CNOT4 |
| UBN2 | ZNF212 |
| UBR5 | UBR5 |
| UBTD1 | BAG3 |
| UBXN4 | DNAJC10 |
| UBXN7 | MSL2 |
| UCHL5 | ADSS |
| UCHL5 | HRSP12 |
| UCHL5 | RAB3GAP2 |
| UCHL5 | TAF5L |
| UEVLD | CTTN |
| UFC1 | UBE2Q1 |
| UFM1 | UFM1 |
| UGGT1 | UGGT1 |
| UIMC1 | C5orf45 |
| UMPS | MRPS22 |
| UPF3A | TFDP1 |
| UPF3B | ZNF280C |
| UPP1 | LGALS3 |
| UPP1 | MET |
| UQCR10 | ACO2 |
| UQCR11 | RNF126 |
| UQCRC2 | MAPRE1 |
| USO1 | G3BP2 |
| USP1 | LRRC40 |
| USP1 | PPIH |
| USP1 | RFC4 |
| USP1 | SNRNP40 |
| USP1 | STIL |
| USP2 | USP2 |
| USP21 | NDUFS2 |
| USP31 | USP31 |
| USP34 | ZNF638 |
| USP36 | GGA3 |
| USP36 | TAOK1 |
| USP37 | SP3 |
| USP42 | ZNF12 |
| USP48 | DNAJC8 |
| USP49 | USP49 |
| USP7 | E4F1 |
| USP7 | PKD1 |
| USP7 | THUMPD1 |
| USP7 | USP7 |
| USP7 | ZNF500 |
| UTP11L | PPIH |
| UTP15 | TAF9 |
| UTP18 | NME1 |
| UTP18 | NME2 |
| UTP23 | DSCC1 |
| UTP23 | UBR5 |
| UTP6 | AATF |
| VBP1 | PHF6 |
| VBP1 | RBMX2 |
| VCP | VCP |
| VCPIP1 | VCPIP1 |
| VHL | WDR48 |
| VN1R1 | VN1R1 |
| VN1R5 | VN1R5 |
| VPS16 | PTPRA |
| VPS26B | THYN1 |
| VPS33B | MAN2C1 |
| VPS37B | ABCB9 |
| VPS39 | VPS39 |
| VPS4A | NARFL |
| VPS72 | PYGO2 |
| VPS72 | SCNM1 |
| VRK1 | MTA1 |
| VRK1 | PAPOLA |
| VRK1 | TOPBP1 |
| VRK3 | VRK3 |
| VSIG10 | SMAGP |
| VTA1 | PCMT1 |
| VTI1B | TMED10 |
| WAC | RBM17 |
| WAPAL | KIF20B |
| WASL | WASL |
| WBP2NL | WBP2NL |
| WBP4 | FAM48A |
| WBP5 | CETN2 |
| WBP5 | WBP5 |
| WDHD1 | EXOC5 |
| WDHD1 | GMNN |
| WDR1 | ADD1 |
| WDR18 | NDUFS7 |
| WDR18 | POLR2E |
| WDR20 | PAPOLA |
| WDR33 | RMND5A |
| WDR36 | CHD1 |
| WDR44 | UBE2A |
| WDR46 | ZBTB9 |
| WDR5 | EHMT1 |
| WDR61 | SEC11A |
| WDR74 | PRPF19 |
| WDR76 | DUT |
| WDR83 | MED26 |
| WDR90 | NFATC2IP |
| WFDC10A | WFDC10A |
| WHSC1L1 | VDAC3 |
| WIBG | WIBG |
| WIPF2 | TMUB2 |
| WRN | CNOT7 |
| WTAP | ADAT2 |
| WWP1 | CPNE3 |
| WWTR1 | TNFRSF1A |
| XPO1 | MSH2 |
| XPO1 | WBP11 |
| XPO4 | CDK8 |
| XPO4 | SLC25A15 |
| XPO5 | SLC29A1 |
| XPO7 | ATP6V1B2 |
| XPO7 | COPS5 |
| XPOT | DDIT3 |
| XRCC1 | LIG1 |
| XRN1 | MSL2 |
| YEAT54 | NFYB |
| YIPF2 | YIPF2 |
| YIPF4 | PNO1 |
| YIPF5 | AP3B1 |
| YIPF5 | CLINT1 |
| YIPF5 | GDE1 |
| YIPF5 | PRKAA1 |
| YIPF5 | RAD17 |
| YIPF5 | YAF2 |
| YLPM1 | DCAF5 |
| YLPM1 | TDP1 |
| YME1L1 | ACBD5 |
| YME1L1 | ATP5C1 |
| YME1L1 | MLLT10 |
| YTHDC1 | ELF2 |
| YTHDC2 | CETN3 |
| YTHDC2 | GIN1 |
| YTHDF2 | HNRNPR |
| YTHDF2 | SLC25A33 |
| YWHAB | RALGAPB |
| YWHAE | TAB2 |
| YWHAZ | ARPC5 |
| YWHAZ | HRSP12 |
| YWHAZ | POLR2K |
| YY1 | PAPOLA |
| YY1AP1 | ASH1L |
| ZAN | GIMAP1 |
| ZBED4 | GTPBP1 |
| ZBED4 | PARP1 |
| ZBED4 | SREBF2 |
| ZBTB1 | EXOC5 |
| ZBTB17 | DNAJC8 |
| ZBTB22 | PPP1R10 |
| ZBTB22 | RXRB |
| ZBTB22 | TJAP1 |
| ZBTB33 | 2NF280C |
| ZBTB4 | TOM1L2 |
| ZBTB4 | ZBTB4 |
| ZBTB41 | ZBTB41 |
| ZBTB44 | ZNF202 |
| ZBTB9 | ZBTB9 |
| ZC3H14 | PPP2R5E |
| ZC3H15 | NCL |
| ZC3H15 | PHKRA |
| ZC3H18 | MON1B |
| ZC3H3 | RECQL4 |
| ZCCHC10 | MATR3 |
| ZCCHC11 | PTBP2 |
| ZCCHC17 | ZCCHC17 |
| ZCCHC24 | VIM |
| ZCCHC8 | BRAP |
| ZDHHC9 | OCRL |
| ZFAND1 | HRSP12 |
| ZFAND1 | UBE2W |
| ZFP1 | TERF2IP |
| ZFP28 | ZFP28 |
| ZFP30 | ZFP28 |
| ZFP30 | ZNF470 |
| ZFP30 | ZNF567 |
| ZFP82 | ZFP28 |
| ZFP82 | ZNF583 |
| ZKSCAN1 | KRIT1 |
| ZKSCAN4 | TRIM27 |
| ZKSCAN5 | KRIT1 |
| ZKSCAN5 | ZC3HC1 |
| ZKSCAN5 | ZNF655 |
| ZMYM4 | PTBP2 |
| ZMYND19 | GTF3C5 |
| ZMYND8 | ZMYND8 |
| ZNF100 | ZNF420 |
| ZNF101 | MED26 |
| ZNF101 | RFXANK |
| ZNF107 | EZH2 |
| ZNF12 | ZNF12 |
| ZNF124 | FLVCR1 |
| ZNF124 | MDM4 |
| ZNF134 | ZNF256 |
| ZNF134 | ZNF419 |
| ZNF142 | NCL |
| ZNF142 | POLR1B |
| ZNF155 | ZNF223 |
| ZNF16 | ZNF696 |
| ZNF174 | ZNF174 |
| ZNF18 | ZNF18 |
| ZNF184 | HMGN4 |
| ZNF189 | ZNF189 |
| ZNF200 | ZNF263 |
| ZNF211 | ZNF211 |
| ZNF212 | CASP2 |
| ZNF212 | EZH2 |
| ZNF212 | ZNF212 |
| ZNF212 | ZNF282 |
| ZNF213 | ZNF213 |
| ZNF22 | ZNF22 |
| ZNF24 | HDHD2 |
| ZNF254 | ZNF430 |
| ZNF254 | ZNF91 |
| ZNF256 | ZNF416 |
| ZNF263 | THOC6 |
| ZNF263 | USP7 |
| ZNF271 | HDHD2 |
| ZNF271 | TXNL1 |
| ZNF273 | EZH2 |
| ZNF273 | HNRNPA2B1 |
| ZNF277 | ZNF277 |
| ZNF282 | REPIN1 |
| ZNF282 | ZNF212 |
| ZNF282 | ZNF282 |
| ZNF292 | SENP6 |
| ZNF300 | ZNF300 |
| ZNF304 | ZNF256 |
| ZNF317 | AKAP8L |
| ZNF317 | UPF1 |
| ZNF320 | ZNF701 |
| ZNF324 | MZF1 |
| ZNF324 | ZNF444 |
| ZNF329 | ZNF829 |
| ZNF335 | SRRT |
| ZNF335 | TNFRSF6B |
| ZNF335 | ZNF611 |
| ZNF337 | NAPB |
| ZNF33A | MLLT10 |
| ZNF33A | ZNF37A |
| ZNF345 | ZFP14 |
| ZNF347 | ZFP82 |
| ZNF347 | ZNF701 |
| ZNF347 | ZSCAN18 |
| ZNF37A | MLLT10 |
| ZNF397 | HDHD2 |
| ZNF398 | EZH2 |
| ZNF398 | NRF1 |
| ZNF398 | REPIN1 |
| ZNF398 | ZNF786 |
| ZNF407 | RTTN |
| ZNF407 | ZNF407 |
| ZNF415 | ZSCAN18 |
| ZNF419 | ZNF416 |
| ZNF428 | SAE1 |
| ZNF43 | ZSCAN18 |
| ZNF430 | ZNF430 |
| ZNF444 | ZNF574 |
| ZNF444 | ZNF611 |
| ZNF445 | CCDC12 |
| ZNF445 | CNOT10 |
| ZNF445 | WDR48 |
| ZNF48 | PRR14 |
| ZNF483 | ZNF483 |
| ZNF493 | ZNF91 |
| ZNF500 | E4F1 |
| ZNF500 | FAM193B |
| ZNF500 | PDPK1 |
| ZNF500 | UBN1 |
| ZNF500 | USP7 |
| ZNF500 | ZNF263 |
| ZNF506 | ZNF91 |
| ZNF510 | PHF2 |
| ZNF510 | ZNP189 |
| ZNF512 | ZNF512 |
| ZNF512B | ZNF512B |
| ZNF519 | ZNF519 |
| ZNF521 | ZNF521 |
| ZNF528 | ZSCAN18 |
| ZNF542 | ZNF542 |
| ZNF548 | ZNF416 |
| ZNF551 | ZNF8 |
| ZNF566 | ZNF235 |
| ZNF566 | ZNF780B |
| ZNF568 | ZFP28 |
| ZNF568 | ZNF470 |
| ZNF568 | ZNF583 |
| ZNF569 | ZFP28 |
| ZNF569 | ZNF331 |
| ZNF569 | ZNF470 |
| ZNF569 | ZNF583 |
| ZNF570 | ZFP28 |
| ZNF570 | ZNF583 |
| ZNF573 | ZNF567 |
| ZNF574 | LIG1 |
| ZNF576 | SAE1 |
| ZNF580 | ZNF574 |
| ZNF581 | C19orf48 |
| ZNF581 | TRIM28 |
| ZNF582 | ZFP28 |
| ZNF582 | ZNF542 |
| ZNF592 | SIN3A |
| ZNF606 | ZNF256 |
| ZNF606 | ZNF419 |
| ZNF609 | ARIH1 |
| ZNF610 | ZNF528 |
| ZNF610 | ZNF71 |
| ZNF610 | ZNF829 |
| ZNF611 | POLD1 |
| ZNF611 | ZNF701 |
| ZNF611 | ZNF83 |
| ZNF639 | TBCCD1 |
| ZNF644 | CCDC76 |
| ZNF644 | GPBP1L1 |
| ZNF644 | MIER1 |
| ZNF644 | PTBP2 |
| ZNF644 | RBMXL1 |
| ZNF653 | RAVER1 |
| ZNF665 | ZNF701 |
| ZNF665 | ZNF91 |
| ZNF669 | ZNF678 |
| ZNF670 | ZNF670 |
| ZNF682 | ZNF420 |
| ZNF684 | ITGB38P |
| ZNF684 | PPIH |
| ZNF684 | STIL |
| ZNF688 | CD2BP2 |
| ZNF688 | PRR14 |
| ZNF689 | MAZ |
| ZNF696 | HSF1 |
| ZNF7 | COMMD5 |
| ZNF7 | HRSP12 |
| ZNF7 | MRPL13 |
| ZNF7 | POLR2K |
| ZNF7 | RNF139 |
| ZNF708 | EZH2 |
| ZNF708 | ZNF101 |
| ZNF708 | ZNF430 |
| ZNF708 | ZNF566 |
| ZNF708 | ZNF91 |
| ZNF71 | ZNF420 |
| ZNF746 | ZNF212 |
| ZNF76 | ZNF76 |
| ZNF767 | EZH2 |
| ZNF767 | ZNF212 |
| ZNF768 | SETD1A |
| ZNF776 | ZNF264 |
| ZNF777 | ZNF282 |
| ZNF780A | ZNF780A |
| ZNF780B | ZNF235 |
| ZNF780B | ZNF780A |
| ZNF786 | EZH2 |
| ZNF786 | PAXIP1 |
| ZNF786 | ZNF212 |
| ZNF786 | ZNF786 |
| ZNF787 | TRIM28 |
| ZNF789 | KRIT1 |
| ZNF829 | ZFP28 |
| ZNF829 | ZNF470 |
| ZNF829 | ZNF583 |
| ZNF83 | ZNF701 |
| ZNF880 | ZSCAN18 |
| ZNHIT1 | PLOD3 |
| ZNRD1 | TAF8 |
| ZNRD1 | ZNRD1 |
| ZNRF1 | ZNRF1 |
| ZNRF2 | ZNRF2 |
| ZRANB2 | PTBP2 |
| ZRANB2 | RNPC3 |
| ZSCAN12 | ZSCAN12 |
| ZSCAN22 | ZSCAN22 |
| ZSWIM1 | DNTTIP1 |
| ZWILCH | CCNB2 |
| ZWINT | MKI67 |
| ZWINT | SUV39H2 |

1. A system for identifying Synthetic Lethal (SL) interactions of pairs of genes in cancer cells, the system comprising:
a non-transitory computer readable memory having stored thereon datasets comprising data related to multiple genes in said cancer cells, and
a processing circuitry configured to recursively:
select a pair of genes comprising a first gene (A) and a second gene (B) from the multiple genes datasets;
analyze the pair of genes to determine the association of said pair of genes, wherein the association is determined by one or more of the following procedures:
examine if an occurrence of co-inactivation in the cancer cells of the first gene and the second gene is lower than a predetermined threshold;
determine if the essentiality of the second gene (B) is higher in the cancer cells in which the first gene (A) is inactive; and/or
determine if the expression of the first gene and the second gene correlate with cancer;
and;
determine, based on said analysis, if the pair of genes interact via an SL-interaction, and/or determine the strength of the SL-interaction.

2. The system of claim 1, wherein the data related to the multiple genes is selected from activity profile of the genes, essentiality profile of the genes, expression profile of the genes, or combinations thereof.

3. The system of claim 2, wherein the activity profile of the genes comprises Somatic Copy Number of Alterations (SCNA), germline Copy-Number Variations (CNV), DNA methylation, histone methylation, somatic mutations, germline mutations or combinations thereof, obtained from a source selected from the group consisting of: a sample obtained from a subject having cancer or suspected to have cancer, a database of cancer patients, a database of cancer cell lines, or combinations thereof.

4. The system of claim 2, wherein the essentiality profile of the genes is determined based on the level of lethality of cells following the inhibition of expression or activity of the genes in the cells.

5. The system of claim 2, wherein the expression profile of the genes comprises a transcriptomic profile or a protein abundance profile of the cells.

6. The system of claim 1, wherein the processing circuitry is further configured to generate an SL-network, based on the pairs of genes identified to interact via SL-interaction and/or on the strength of the SL-interaction between each pair.

7. The system of claim 6, wherein the processing circuitry is further configured to determine an occurrence selected from the group consisting of:
v. response of cancer cells to the inhibition of a gene product;
vi. survival of a subject having cancer;
vii. response of cancer cells to a specific drug; and
viii. ranking of cancer treatments for a specific subject having cancer;
comprising applying the identified SL-network on a genomic profile of cells, wherein the genomic profile of cells is obtained from a subject, a population of subjects, a genomic dataset, cancer cells of at least one subject, or any combination thereof.

8. The system of claim 7, wherein the survival of the subject having cancer is inversely-correlated to the number of the SL-paired genes which are co-inactive in the subject's tumor based on the determined SL-network and the genomic profile of the subject's tumor; or wherein the presence of co-underexpressed SL-paired genes in the subject correlates with improved prognosis of survival of the subject having cancer compared to other subjects afflicted with cancer.

9. The system of claim 7, wherein the prediction of response of cancer cells to the inhibition of a gene product is utilized using a supervised mode or an unsupervised mode.

10. A system for identifying Synthetic Dosage Lethal (SDL)-interactions of pairs of genes in cancer cells, the system comprising:
a non-transitory computer readable memory having stored thereon datasets comprising data related to multiple genes in said cancer cells, and
a processing circuitry configured to recursively:
select a pair of genes comprising a first gene (A) and a second gene (B) from the multiple genes datasets;
analyze the pair of genes to determine an association of said pair of genes, wherein the association is determined by one or more of the following procedures:
examine if an occurrence of over activation in the cancer cells of the first gene and inactivation of the second gene is lower than a predetermined threshold;
determine if the essentiality of the second gene (B) is higher in the cancer cells in which the first gene (A) is overactive; and/or
determine if the expression of the first gene and the second gene correlate with cancer;
and;
determine, based on said score, if the pair of genes interact via an SDL-interaction, and/or determine the strength of the SDL-interaction.

11. The system of claim 10, wherein the data related to the multiple genes is selected from activity profile of the genes, essentiality profile of the genes, expression profile of the genes, or combinations thereof.

12. The system of claim 11, wherein the activity profile of the genes comprises Somatic Copy Number of Alterations (SCNA), germline Copy-Number Variations (CNV), DNA methylation, histone methylation, somatic mutations, germline mutations or combinations thereof, obtained from a source selected from the group consisting of: a sample obtained from a subject having cancer or suspected to have cancer, a database of cancer patients, a database of cancer cell lines, or combinations thereof.

13. The system of claim 11, wherein the essentiality profile of the genes is determined based on the level of lethality of cells following the inhibition of expression or activity of the genes in the cells.

14. The system of claim 11, wherein the expression profile of the genes comprises a transcriptomic profile or a protein abundance profile of the cells.

15. The system of claim 10, wherein the processing circuitry is further configured to generate an SDL-network, based on the pairs of genes identified to interact via SDL-interaction and/or on the strength of the SDL-interaction between each pair.

16. The system of claim 15, wherein the processing circuitry is further configured to determine an occurrence selected from the group consisting of:
i. response of cancer cells to the inhibition of a gene product;
ii. survival of a subject having cancer;
iii. response of cancer cells to a specific drug; and
iv. ranking of cancer treatments for a specific subject having cancer;
comprising applying the identified SDL-network on a genomic profile of cells, wherein the genomic profile of cells is obtained from a subject, a population of subjects, a genomic dataset, cancer cells of at least one subject, or any combination thereof.

17. The system of claims 16, wherein the prediction of response of cancer cells to the inhibition of a gene product is utilized using a supervised mode or an unsupervised mode.

18. The system of claim 1, used in a method of repurposing an active ingredient for use in cancer therapy, the method comprising applying the SL-network on a genomic profile of cells, to identify the active ingredient as candidate for targeting an identified SL gene.

19. The system of claim 10, used in a method of repurposing an active ingredient for use in cancer therapy, the method comprising applying the SDL-network on a genomic profile of cells, to identify the active ingredient as candidate for targeting an identified SDL gene.

20. A method of treating cancer comprising administering to a subject in need thereof a pharmaceutical composition comprising at least one active ingredient identified as a candidate for targeting an identified SL gene or SDL gene.

21. The method of claim 20, wherein the pharmaceutical composition comprises at least one active ingredient selected from the group consisting of: Pentolinium, Imipramine, Dalfampridine, Amitriptyline, Verapamil and Dronedarone.

22. The method of claim 20, wherein the cancer is VHL-deficient cancer.
